(19) United States
(12) Patent Application Publication
(10) Pub. No.: US 2021/0277351 A1
(43) Pub. Date: Sep. 9, 2021

(54) METHODS FOR HETEROTROPHICALLY CULTURING EUGLENA IN HYBRID MEDIA

(71) Applicant: NOBLEGEN INC., Peterborough (CA)

(72) Inventors: Yuan LIU, Peterborough (CA); Alexandra Margaret KUHNE, Peterborough (CA); Adam J. NOBLE, Peterborough (CA); Erin Nicole MORRISON, Peterborough (CA)

(21) Appl. No.: 17/253,183

(22) PCT Filed: Jun. 28, 2019
(86) PCT No.: PCT/IB2019/055524

§ 371 Date: Dec. 17, 2020

Related U.S. Application Data
(60) Provisional application No. 62/692,528, filed on Jun. 29, 2018.

(51) Int. Cl. C12N 1/12; C12P 7/48; C12P 7/62; C12P 7/46; C12P 7/58; C12P 7/54; C12P 7/56

(52) U.S. Cl. CPC C12N 1/12; C12P 7/48; C12P 7/625; C12P 7/56; C12P 7/62; C12P 7/58; C12P 7/54; C12P 7/46

(57) ABSTRACT

The present application discloses a method of culturing a Euglena sp. microorganism, Schizochytrium sp. microorganism, or a Chlorella sp. microorganism comprising culturing the microorganism in a hybrid culture media; maintaining the microorganism heterotrophically in an environment substantially free from light; wherein the hybrid culture media comprises a carbohydrate; and wherein the hybrid culture media comprises fresh media and recycled culture media. The present application also discloses a hybrid culture media.

### FIELD

The present application relates to methods for heterotrophically culturing Euglena. For example, the present application relates to methods and uses of hybrid culture media for heterotrophically growing Euglena.

### BACKGROUND

Euglena gracilis belongs to a group of single-celled microscopic algae that is used as a model species in laboratory studies and also have industrial applications in producing biofuel, food and beverage products. Euglena possesses the representative features typical of eukaryotic cells such as a mitochondria, nucleus, and lysosome (Gomez et al., “Studies of Euglena gracilis in Aging Cultures. I. Light Microscopy and Cytochemistry,” Br. Phycol. J. 9:163-74 (1974); Kings et al., “Growth Studies on Microalgae Euglena sanguinea in Various Natural Eco-Friendly Composite Media to Optimize the Lipid Productivity,” Bioresour. Technol. 244:1349-57 (2017)). Euglena can further be characterized for its long flagellum and large red eyespot (Kings et al., “Growth Studies on Microalgae Euglena sanguinea in Various Natural Eco-Friendly Composite Media to Optimize the Lipid Productivity,” Bioresour. Technol. 244:1349-57 (2017)). They are distinctive as they can produce their own nourishment (autotrophic) similar to plants, as well as taking up external food sources (heterotrophic) like animals (Gomez et al., “Studies of Euglena gracilis in Aging Cultures. I. Light Microscopy and Cytochemistry,” Br. Phycol. J. 9:163-74 (1974)). Through optimizing the natural ability to employ singly or both modes of nourishment, Euglena may be directed to produce target compounds by adjusting key parameters in the production process (Acién Fernández et al., “Photobioreactors: Light Regime, Mass Transfer, and Scaleup,” Progress in Industrial Microbiology 35:231-47 (1999)). These adjustments may be used to improve efficiency of converting input nutrient source to encourage rapid growth while minimizing waste production.

Nutrient and media recycling has been suggested for microalgae growth in heterotrophic culture, which may mitigate waste production and reduce costs (Lowrey et al., “Nutrient and Media Recycling in Heterotrophic Microalgea Cultures,” Appl. Microbiol. Biotechnol. 100(3):1061-75 (2016)). However, there are many technical and practical challenges associated with nutrient recycling for heterotrophic microalgae, including the formation of inhibitory compounds such as heavy metals, phenols, fatty acids, and ammonia that may adversely affect microalgae (Lowrey et al., “Nutrient and Media Recycling in Heterotrophic Microalgea Cultures,” Appl. Microbiol. Biotechnol. 100(3):1061-75 (2016)). Further, the accepted technical step of sterilization of recycled culture media incurs additional costs, and heat sterilization of “waste products” can even generate unwanted compounds adverse to microalgae growth (Lowrey et al., “Nutrient and Media Recycling in Heterotrophic Microalgea Cultures,” Appl. Microbiol. Biotechnol. 100(3):1061-75 (2016)).

Of interest, phytohormones can be an indicator of culture health as they are involved in cell signaling and cell growth. Phytohormones are traditionally thought of as plant growth regulators, but have been found in bacteria, fungi, mammals and algae. Microalgae possess functional but relatively simple phytohormone biosynthetic pathways that are equivalent to those in higher plants. In microalgae, phytohormones appear to play a regulatory role in cell development, including cell division or elongation and in chlorophyll and protein metabolism (Pei et al., “Toward Facilitating Microalgae Cope With Effluent from Anaerobic Digestion of Kitchen Waste: the Art of Agricultural Phytohormones,” Biotech Biofuels 10:76 (2017)). Phytohormones may enhance tolerance to stress stimulating algae to deal with unfavorable conditions (Pei et al., “Toward Facilitating Microalgae Cope With Effluent from Anaerobic Digestion of Kitchen Waste: the Art of Agricultural Phytohormones,” Biotech Biofuels 10:76 (2017)). Phytohormones or hormone analogs have been highlighted as potentially valuable cultivation additives in promoting algal growth and metabolite biosynthesis (Liu et al., “The Boosted Biomass and Lipid Accumulation in Chlorella vulgaris by Supplementation of Synthetic Phytohormone Analogs,” Bioresource Technol. 232:44-52 (2017); Han et al., “Phytohormones and Effects on Growth and Metabolites of Microalgae: A Review,” Fermentation 4(2):25 (2018)). Detection of phytohormones and positive growth regulators in the recycled media of Euglena gracilis cultured cells presents an opportunity for use in continuous culturing as well as potential use as a growth enhancer for algae as well as plant systems.

Cytokinins (CKs) and abscisic acid (ABA) are structurally distinct molecules that exist at low concentrations and can impact the growth and development of algae (Han et al., “Phytohormones and Effects on Growth and Metabolites of Microalgae: A Review,” Fermentation 4(2):25 (2018)). In algae, CKs activate cell cycle and also influence metabolite production including carotenoids, lipid, carbohydrate and proteins (Salama et al., “Enhancement of Microalgae Growth and Fatty Acid Content Under the Influence of Phytohormones,” Bioresource Technology 172:97-103 (2014); Han et al., “Phytohormones and Effects on Growth and Metabolites of Microalgae: A Review,” Fermentation 4(2):25 (2018)). ABA serves as a growth inhibitor and stimuli to induce stress tolerance (Han et al., “Phytohormones and Effects on Growth and Metabolites of Microalgae: A Review,” Fermentation 4(2):25 (2018)). ABA in microalgae has been found to increase biomass production, carotenogenesis and lipid biosynthesis (Kozlova et al., “Effect of Phytohormones on Growth and Accumulation of Pigments and Fatty Acids in the Microalgae Scenedesmus quadricauda,” Algal Research 27:325-34 (2017)). Phytohormones can simultaneously promote both microalgal cell growth and end product biosynthesis (Kozlova et al., “Effect of Phytohormones on Growth and Accumulation of Pigments and Fatty Acids in the Microalgae Scenedesmus quadricauda,” Algal Research 27:325-34 (2017)). Knowledge about the metabolic and regulatory networks of microalgal phytohormones provides new opportunities for microalgal cultivation and application (Liu et al., “Extracellular Metabolites from Industrial Microalgae and Their Biotechnological Potential,” Mar. Drugs 14(10):191 (2016))

The present invention is directed to overcoming these and other deficiencies in the art.

### SUMMARY

Accordingly, the present application includes a method of culturing a Euglena sp. microorganism, a Schizochytrium sp. microorganism, or a Chlorella sp. microorganism comprising:
culturing the microorganism in a hybrid culture media;
maintaining the microorganism heterotrophically in an environment substantially free from light;
wherein the hybrid culture media comprises a carbon source, optionally a carbohydrate; and
wherein the hybrid culture media comprises fresh media and recycled culture media.

In an embodiment, the microorganism is inoculated at about 1×10⁵ cells/mL to about 5×10⁷ cells/mL.

In an embodiment, the microorganism is inoculated at about 0.5 g/L to about 150 g/L dry cell weight.

In another embodiment, the recycled culture media is obtained by separating the recycled culture media from a source culture media, wherein the source culture media is in a lag phase, an exponential phase, or a stationary phase.

In another embodiment, the microorganism is grown for about 4 hours to about 350 hours, or up to about 75 days.

In another embodiment, the microorganism is grown for at least 2, 3, 4, 5, 6, 7, 8, 9, 10, 11, 12, 13, 14, 15, 16, 17, 18, 19, 20, 25, 30, 35, 40, 45 or 50 cycles.

In another embodiment, the method is batch, fed-batch, semi-continuous or continuous.

In another embodiment, the method is fed-batch, semi-continuous or continuous.

In another embodiment, the method is semi-continuous or continuous.

In another embodiment, recycled culture media is added to the hybrid culture media at lag, exponential or stationary phase.

In another embodiment, the recycled culture media is selected from the group consisting of a culture media, a feed media, a spent media media, a supplemented media, and combinations thereof, optionally a spent media or a supplemented media.

In another embodiment, the microorganism is harvested at lag, exponential, or stationary phase.

In another embodiment, the harvested microorganism are separated from the media and the media is recycled back into the culture.

In another embodiment, the media recycled back into the culture is spent media.

In another embodiment, the spent media comprises total carbohydrate of less than about 3, 2.5, 2, 1.5, 1, 0.5, 0.4, 0.3, 0.2, 0.1, 0.01 g/L.

In another embodiment, the carbohydrate is glucose, fructose, galactose, lactose, maltose, sucrose, molasses, glycerol, xylose, dextrose, honey, and/or corn syrup.

In another embodiment, about 10% to about 75% of the spent or recycled culture media is returned to the culture, optionally about 25% to about 75%, optional about 50% to about 75%, optionally about 75%.

In another embodiment, the microorganism is selected from the group consisting of Euglena gracilis, Euglena sanguinea, Euglena deses, Euglena mutabilis, Euglena acus, Euglena virdis, Euglena anabaena, Euglena geniculata, Euglena oxyuns, Euglena proxima, Euglena tipters, Euglena chiamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena vaiabilis, Euglena caudata, Euglena minima, Euglena communis, Euglena magnifica, Euglena terricola, Euglena velata, Euglena repulsans, Euglena clavata, Euglena lata, Euglena tuberculata, Euglena contabrica, Euglena ascusformis, Euglena ostendensis, Chlorella autotrophica, Chlorella colonials, Chlorella lewinii, Chlorella minutissima, Chlorella pituita, Chlorella pulchelloides, Chlorella pyrenoidosa, Chlorella rotunda, Chlorella singulans, Chlorella sorokiniana, Chlorella variabilis, Chlorella volutis, Chlorella vulgaris, Schizochytrium aggregatum, Schizochytrium limacinum, Schizochytrium minutum, and combinations thereof.

In another embodiment, the method does not comprise phototrophic culturing.

In another embodiment, the method does not comprise hybrid media sterilization.

In another embodiment, the microorganism is Euglena and the method of culturing Euglena produces about 0.01 pmol/mL to about 100 nmol/mL, optionally 2 nmol/mL to about 100 nmol/mL cytokinins, optionally about 2 nmol/mL to about 50 nmol/mL cytokinins, optionally about 2 nmol/mL to about 25 nmol/mL cytokinins, optionally about 2 nmol/mL to about 15 nmol/mL cytokinins.

In another embodiment, the microorganism is Euglena and the method of culturing Euglena produces about 0.01 pmol/mL to about 100 nmol/mL cytokinins, optionally about 0.01 pmol/mL to about 0.25 nmol/mL cytokinins, optionally about 0.01 pmol/mL to about 0.50 nmol/mL cytokinins, optionally about 0.01 pmol/mL to about 0.75 nmol/mL cytokinins, optionally about 0.01 pmol/mL to about 1 nmol/mL cytokinins, optionally about 0.01 pmol/mL to about 1.5 nmol/mL cytokinins, optionally about 0.01 pmol/mL to about 2 nmol/mL cytokinins, optionally about 0.01 pmol/mL to about 30 nmol/mL cytokinins, optionally about 0.01 pmol/mL to about 50 nmol/mL cytokinins, optionally about 0.05 nmol/mL to about 75 nmol/mL cytokinins, optionally about 0.05 nmol/mL to about 50 nmol/mL cytokinins, optionally about 0.05 nmol/mL to about 40 nmol/mL cytokinins, optionally about 0.05 nmol/mL to about 35 nmol/mL cytokinins, optionally about 0.05 nmol/mL to about 30 nmol/mL cytokinins, optionally about 0.05 nmol/mL to about 25 nmol/mL cytokinins, optionally about 0.05 nmol/mL to about 20 nmol/mL cytokinins, optionally about 0.05 nmol/mL to about 15 nmol/mL cytokinins, optionally about 0.05 nmol/mL to about 10 nmol/mL cytokinins, optionally about 0.05 nmol/mL to about 5 nmol/mL cytokinins, optionally about 0.05 nmol/mL to about 4 nmol/mL cytokinins, optionally about 0.05 nmol/mL to about 3 nmol/mL cytokinins, optionally about 0.05 nmol/mL to about 2.5 nmol/mL cytokinins, optionally about 0.05 nmol/mL to about 2 nmol/mL cytokinins, optionally about 0.05 nmol/mL to about 1.5 nmol/mL cytokinins, optionally about 0.05 nmol/mL to about 1 nmol/mL cytokinins, optionally about 0.05 nmol/mL to about 0.5 nmol/mL cytokinins, optionally about 0.05 nmol/mL to about 0.1 nmol/mL cytokinins, optionally about 0.05 nmol/mL to about 25 nmol/mL cytokinins.

In another embodiment, the microorganism is Euglena and the method of culturing Euglena produces about 0.01 pmol/mL to about 100 nmol/mL ABA.

In another embodiment, the microorganism is Euglena and the method of culturing Euglena produces about 0.000005 g/L to about 20 g/L of an organic acid selected from the group consisting of citric acid, citrate, fumaric acid, fumarate, malic acid, malate, pyruvic acid, pyruvate, succinic acid, succinate, acetic acid, acetate, lactic acid, lactate, and combinations thereof.

In another embodiment, the culture media is maintained at a pH of between about 2.5 to about 5, optionally between about 2.5 to about 4.

In another embodiment, the culture media maintains a relative conversion efficiency of at least 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%.

Also provided a method for producing a recycled culture media suitable for heterotrophically culturing a Euglena sp. microorganism, Schizochytrium sp. microorganism or a Chlorella sp. microorganism, comprising:
culturing the microorganism in a culture media comprising a carbohydrate;
maintaining an environment with substantially, or entirely no light;
producing recycled culture media;
separating recycled culture media from cells; and
collecting recycled culture media;
wherein the microorganism is cultured until the carbohydrate is below 3 g/L;
wherein the microorganism is Euglena gracilis;
wherein the recycled culture media is obtained by separating the recycled culture media from a source culture media;
wherein the source culture media is in a lag phase, an exponential phase, or a stationary phase; and
wherein the source culture media is a hybrid culture media or a stock culture media.

In another embodiment, the recycled culture media comprises total carbohydrate of less than about 3, 2.5, 2, 1.5, 1, 0.5, 0.4, 0.3, 0.2, 0.1, 0.01 g/L. In another embodiment, the carbohydrate is glucose, fructose, galactose, lactose, maltose, sucrose, molasses, glycerol, xylose, dextrose, honey, corn syrup, or combinations thereof.

Also provided is a culture media comprising a hybrid culture media, wherein the hybrid culture media comprises a carbohydrate; and wherein the hybrid culture media comprises fresh media and recycled culture media. In another embodiment, the carbohydrate is glucose, fructose, galactose, lactose, maltose, sucrose, molasses, glycerol, xylose, dextrose, honey, corn syrup, or combinations thereof. In another embodiment, the culture media comprises about 0.01 pmol/mL to about 100 nmol/mL cytokinins and/or about 0.01 pmol/mL to about 100 nmol/mL of ABA. Phytohormone sources include media sources and production and excretion from Euglena biomass. In another embodiment, the culture media comprises about 0.000005 g/L to about 20 g/L of an organic acid selected from the group consisting of citric acid, citrate, fumaric acid, fumarate, malic acid, malate, pyruvic acid, pyruvate, succinic acid, succinate, acetic acid, acetate, lactic acid, lactate, and combinations thereof. In another embodiment, the hybrid culture media comprises about 10%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% spent media or recycled culture media.

Also provided is a use of the culture media, spent media, recycled culture media, supplemented media or hybrid culture media produced by the method described herein for culturing a microorganism. In an embodiment, the microorganism is a Euglena sp. In an embodiment, the Euglena sp. is Euglena gracilis.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole. In addition, preferences and options for a given aspect, feature, embodiment, or parameter of the invention should, unless the context indicates otherwise, be regarded as having been disclosed in combination with any and all preferences and options for all other aspects, features, embodiments, and parameters of the invention.

### BRIEF DESCRIPTION OF THE DRAWINGS

The present application will now be described in greater detail with reference to the drawings, in which:

FIG. 1 shows experimental design of control media and Recycle Rate media A-C.

FIGS. 2A and 2B show (FIG. 2A) wet and (FIG. 2B) dry cell weight for different recycled culture media rates. Three different recycle rates (A-C) were tested and compared to a fresh media control (control: 100%) over time (hours). Each time point represents a cycle of growth (i.e. Cycles 1-4, 48-264 hours). FIG. 2A shows the wet cell weight values and FIG. 2B shows dry cell weight values.

FIG. 3 shows dry supernatant weight. Three different recycle rates (Rate A-C) were tested and compared to a fresh media control (control: 100%) over time (hours). Each time point represents a cycle of growth (i.e. Cycles 1-4, 48-264 hours). Dried supernatant weight represents the remaining solutes left in the media.

FIG. 4 shows cell counts over time (cycles). Three different recycle rates (Rate A-C) were tested and compared to a fresh media control (control: 100%) over time (hours). Each time point represents a cycle of growth (i.e. Cycles 1-4, 48-264 hours).

FIGS. 5A and 5B show (FIG. 5A) sugar consumption and (FIG. 5B) pH tracking for different recycled culture media rates. Three different recycle rates (A-B) were tested and compared to a fresh media control (control: 100%) over time (hours). Each time point represents a cycle of growth (i.e. Cycles 1-4, 48-264 hours). FIG. 5A shows glucose consumption at each cycle, and FIG. 5B shows pH changes for each rate and cycle.

FIGS. 6A-6C show Abscisic Acid (ABA) hormone levels in different samples. FIG. 6A shows level of ABA in the media blank. FIG. 6B shows levels of ABA in Cycle 1 at the different conditions tested i.e. control (Ctrl, 100% fresh media), Recycle Rate A (A, hybrid culture media-25%), Recycle Rate B (B, hybrid culture media-50%), and Recycle Rate C (C, hybrid culture media-75%). FIG. 6C represents the ABA levels during the last cycle, Cycle 4.

FIGS. 7A and 7B show total cytokinin levels in the media blank. FIG. 7A shows level of total cytokinin in the media blank. FIG. 7B shows the types of cytokinin in the media blank where: FB represents free bases, RB ribosides, NT nucleotides, Met Methylthiols and Glucs Glucosides.

FIGS. 8A and 8B show total cytokinin (CK) levels in the supernatant in all recycled rates. Error bars represent the standard error for each sample. Concentration for each CK is given on the y-axis in pmol/mL. FIG. 8A represents the total levels of CKs at the beginning (DPI 0) and end (DPI 2) of cycle 1. FIG. 8B represents the Total levels of CKs at the beginning (DPI 0) and end (DPI 2) of cycle 4.

FIGS. 9A and 9B show cytokinin (CK) forms detected in the supernatant of various recycle rates (see Tables 2 and 3). Error bars represent the standard error for each sample. Concentration for each CK is given on the y-axis in pmol/mL. FIG. 9A represents the CK forms at the beginning (DPI 0) and end (DPI 2) of cycle 1. FIG. 9B represents the CK forms at the beginning (DPI 0) and end (DPI 2) of cycle 4. FB represents free bases, RB ribosides, NT nucleotides, Met Methylthiols and Gluc Glucosides.

FIGS. 10A and 10B show total cytokinin levels detected in the pellet of various recycle rates. Error bars represent the standard error for each sample. Concentration for each CK is given on the y-axis in pmol/g. FIG. 10A represents the total levels of CKs at the beginning (DPI 0) and end (DPI 2) of cycle 1. FIG. 10B represents the total levels of CKs at the beginning (DPI 0) and end (DPI 2) of cycle 4.

FIGS. 11A and 11B show cytokinin forms detected in the pellet of various recycle rates. Error bars represent the standard error for each sample. Concentration for each CK is given on the y-axis in pmol/g. FIG. 11A represents the CK forms at the beginning (DPI 0) and end (DPI 2) of cycle 1. FIG. 11B represents the CK forms at the beginning (DPI 0) and end (DPI 2) of cycle 4. FB represents free bases, RB ribosides, NT nucleotides, Met Methylthiols and Gluc Glucosides.

FIG. 12 shows cycling scheme for Condition 1.

FIG. 13 shows cycling scheme for Condition 2.

FIG. 14 shows cycling scheme for Condition 3.

FIG. 15 shows cycling scheme for Condition 4.

FIGS. 16A and 16B show growth curve (FIG. 16A) and glucose consumption (FIG. 16B) for Cycle 1. 1-4 represents Conditions 1 to 4, respectfully. Cell growth is defined as the mass of the dried cell weight for a 5 mL sample of culture over time (days) (FIG. 16A). Glucose consumption of Conditions 1-4, overtime (FIG. 16B).

FIGS. 17A and 17B show growth curve (FIG. 17A) and glucose consumption (FIG. 17B) for Cycle 2. 1-4 represents Conditions 1 to 4 respectfully. Cell growth is defined as the mass of the dried cell weight for a 5 mL sample of culture over time (days) (FIG. 17A). Glucose consumption of Conditions 1-4, overtime (FIG. 17B).

FIGS. 18A and 18B show growth curve (FIG. 18A) and glucose consumption (FIG. 18B) for Cycle 3. 1-4 represents Conditions 1 to 4 respectfully. Cell growth is defined as the mass of the dried cell weight for a 5 mL sample of culture over time (days) (FIG. 18A). Glucose consumption of Conditions 1-4, overtime (FIG. 18B).

FIG. 19 shows cell counts over time with a 4-day growth cycle. A 75% recycled media rate (75% spent media with 25% fresh growth media) was used to test the length of culture time (cycle) used. Cycle 0 represents the growth of the initial growth to generate the recycled media.

FIG. 20 shows cell counts over time with a 3-day growth cycle. A 75% recycled media rate (75% spent media with 25% fresh growth media) was used to test the length of culture time (cycle) used. Cycle 0 represents the growth of the initial growth to generate the recycled media.

FIG. 21 is a graph that represents culture growth for Cycle 1. Bars represent dried biomass (g/mL) taken at time 0 and time 3, while lines represent the cell count (millions/mL). The x-axis represents time of culture over days. Darkest bars represent 100% fresh growth media; medium gray bars represent the 50% recycled hybrid media; and light grey bars represents the glucose supplemented 50% recycled media. Solid black line represents 100% fresh growth media, large dash line represents 50% recycled hybrid media, while small dash line represents glucose supplemented 50% recycled hybrid media.

FIG. 22 is a graph that represents culture growth for Cycle 2. Bars represent dried biomass (g/mL) taken at time 0 and time 3, while lines represent the cell count (millions/mL). The x-axis represents time of culture over days. Darkest bars represent 100% fresh growth media; medium gray bars represent the 50% recycled hybrid media; and light grey bars represents the glucose supplemented 50% recycled hybrid media. Solid black line represents 100% fresh growth media, large dash line represents 50% recycled hybrid media, while small dash line represents glucose supplemented 50% recycled hybrid media.

FIG. 23 is a graph that represents culture growth for Cycle 3. Bars represent dried biomass (g/mL) taken at time 0 and time 3, while lines represent the cell count (millions/mL). The x-axis represents time of culture over days. Darkest bars represent 100% fresh growth media; medium gray represent the 50% recycled hybrid media; and light grey represents the glucose supplemented 50% recycled hybrid media. Solid black line represents 100% fresh growth media, large dash represents 50% recycled hybrid media, while small dash represents glucose supplemented 50% recycled hybrid media.

FIGS. 24A-24C shows the pH changes for each condition for each cycle. Dark grey bars represents 100% fresh growth media, dotted bars represent 50% fresh growth media, medium grey bars represent glucose supplemented 50% fresh growth media, striped bars represent 50% recycled hybrid media and light grey bars represent glucose supplemented 50% recycled hybrid media. FIG. 24A represents cycle 1; FIG. 24B represents cycle 2; and FIG. 24C represents cycle 3. The x-axis represents the days of the cycle where the y-axis represents the pH.

FIG. 25 is a bar graph showing an organic acid profile for 100% fresh media. Organic acids pyruvate, malate, succinic, lactic, fumaric, and acetic are tested in the 100% fresh media control. Legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D)) and Cycle 3, Day 3 (C3D3). Error bars represent the standard error for each sample. Concentration for each organic acid is given on the y-axis in g/L.

FIG. 26 is a bar graph showing an organic acid profile for 50% recycled media. Organic acids pyruvate, malate, succinic, lactic, fumaric, and acetic are tested in the 50% recycled media sample. Legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for each organic acid is given on the y-axis in g/L.

FIG. 27 is a bar graph showing an organic acid profile for the glucose supplemented 50% recycled media. Organic acids pyruvate, malate, succinic, lactic, fumaric, and acetic are tested in the glucose supplemented 50% recycled media sample. Legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D)) and Cycle 3, Day 3 (C3D3). Error bars represent the standard error for each sample. Concentration for each organic acid is given on the y-axis in g/L.

FIG. 28 is a bar graph showing an organic acid profile for the 100% fresh growth media no cell control. Organic acids pyruvate, malate, succinic, lactic, fumaric, and acetic are tested in the 100% fresh growth media no cell control. Legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for each organic acid is given on the y-axis in g/L.

FIG. 29 is a bar graph showing an organic acid profile for the 50% recycled media no cell control. Organic acids pyruvate, malate, succinic, lactic, fumaric, and acetic are tested in the 50% recycled media no cell control. Legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for each organic acid is given on the y-axis in g/L.

FIG. 30 is a bar graph representing media profile of acetic acid. Treatment type is displayed on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for acetic acid is given on the y-axis in g/L.

FIG. 31 is a bar graph representing media profile of fumaric acid. Treatment type is on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for Fumaric acid is given on the y-axis in g/L.

FIG. 32 is a bar graph representing media profile of lactic acid. Treatment type is on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for Lactic acid is given on the y-axis in g/L.

FIG. 33 is a bar graph representing media profile of succinic acid. Treatment type is on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for Succinic acid is given on the y-axis in g/L.

FIG. 34 is a bar graph representing media profile of malate. Treatment type is on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for Malate is given on the y-axis in g/L.

FIG. 35 is a bar graph representing media profile of pyruvic acid. Treatment type is on the x-axis where 100% represents the 100% fresh media control. 50% represents the 50% recycled media, G50% represents the glucose supplemented 50% recycled media, 100% no cell represents the 100% fresh growth media no cell control and 50% no cell represents the 50% recycled media no cell control. The legend represents Cycle 1 day 0 (C1D0), Cycle 1 Day 3 (C1D3), Cycle 2 Day 0 (C2D0), Cycle 2 Day 3 (C2D3), Cycle 3 day 0 (C3D0) and Cycle 3, Day 3 (C3D3). Error bars represent the standard error for each sample. Concentration for Pyruvic Acid is given on the y-axis in g/L.

FIG. 36 is a graph that represents culture growth for Example 3b. Bars represent dried biomass (g/mL) taken at time 0 and time 3, while lines represent the cell count (millions/mL). The x-axis represents time of culture over days. Darkest bars represent 100% fresh growth media; dotted bars represent the 50% fresh growth media; medium gray bars represent the glucose supplemented 50% fresh media; striped bars represent 50% recycled hybrid media; and light grey bars represents the glucose supplemented 50% recycled hybrid media. The Solid black line represents 100% fresh growth media, the large dashed line represents 50% fresh growth media, the dash with a circle line represents the glucose supplemented 50% fresh media, the dash with a triangle line represents 50% recycled hybrid media and the dash with a square line represents glucose supplemented 50% recycled hybrid media cell counts (in millions/mL) over time.

FIG. 37 is a bar graph representing the pH change over time (days). Darkest bars represent 100% fresh growth media pH, dotted bars represent the 50% fresh growth media pH, medium gray bars represent the glucose supplemented 50% fresh media pH, striped bars represent 50% recycled hybrid media pH, and light grey bars represents the glucose supplemented 50% recycled hybrid media pH.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Where a range of values is provided, it is intended that each intervening value between the upper and lower limit of that range and any other stated or intervening value in that stated range is encompassed within the disclosure. For example, if a range of 1 mL to 8 mL is stated, it is intended that 2 mL, 3 mL, 4 mL, 5 mL, 6 mL, and 7 mL are also explicitly disclosed, as well as the range of values greater than or equal to 1 mL and the range of values less than or equal to 8 mL.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

In embodiments comprising an “additional” or “second” component, the second component as used herein is different from the other components or first component. A “third” component is different from the other, first, and second components, and further enumerated or “additional” components are similarly different.

The term “heterotrophic” or derivatives, as used herein, refers to an organism, such as an microorganism including Euglena, which is under conditions such that it obtains nutrients substantially entirely from exogenous sources of organic carbon, such as carbohydrates, lipids, alcohols, carboxylic acids, sugar alcohols, proteins, or combinations thereof. For example, Euglena is a heterotroph where it is in an environment where there is substantially no light.

The term “phototrophic” or derivatives, as used herein, refers to an organism, such as an microorganism including Euglena, when it is under a condition that it can carry out photon capture to acquire energy. For example, when an organism is phototrophic, it carries out photosynthesis to produce energy.

The term “mother culture” as used herein refers to a culture of cells that is continuously grown over time with media and cells removed or replenished on a schedule independent of the experimental conditions described herein.

Certain terms employed in the specification, examples and claims are collected herein. Unless defined otherwise, all technical and scientific terms used in this disclosure have the same meanings as commonly understood by one of ordinary skill in the art to which this disclosure belongs.

The present disclosure includes methods for culturing a microorganism heterotrophically in a culture media, wherein the culture media comprises recycled culture media.

Accordingly, the present application includes a method of culturing a Euglena sp. microorganism, Schizochytrium sp. microorganism, or a Chlorella sp. microorganism comprising:
culturing the microorganism in a hybrid culture media;
maintaining the microorganism heterotrophically in an environment substantially, or entirely free from light;
wherein the hybrid culture media comprises a carbohydrate; and
wherein the hybrid culture media comprises fresh media and recycled culture media.

In an embodiment, the method comprises maintaining the microorganism heterotrophically in an environment substantially free from light. In another embodiment, the method comprises maintaining the microorganism heterotrophically in an environment entirely free from light.

Microalgae is grown in culture media that includes one or more carbon sources, one or more nitrogen sources, and one or more salt sources. In some embodiments growth media may also include exogenous nutrients and/or additives such as carbohydrates, lipids, alcohols, carboxylic acids, sugar alcohols, proteins, nitrogen, metals, vitamins, minerals, or combinations thereof.

The microorganism is inoculated at a range of concentrations. In an embodiment, the microorganism is inoculated at about 1×10⁵ cells/mL to about 5×10⁷ cells/mL, optionally at about 1×10⁵ cells/mL to about 1×10⁷ cells/mL, optionally at about 2×10⁵ cells/mL to about 5×10⁶ cells/mL, optionally about 2.5×10⁵ cells/mL to about 3×10⁶ cells/mL.

The microorganism can also be inoculated at a range of dry cell weights. In an embodiment, the microorganism is inoculated at about 0.5 g/L to about 70 g/L, optionally at about about 0.5 to about 30 g/L, optionally at about 0.5 to about 70 g/L, optionally at about 1.0 g/L to about 70 g/L, optionally at about 1.0 g/L to about 30 g/L, optionally at about 1.0 to about 150 g/L.

Growth of microorganisms in a culture undergoes different phases: lag phase, log (logarithmic) phase or exponential phase, stationary phase, and death phase. During lag phase, microorganisms are maturing and metabolically active but not actively dividing or reproducing. During log phase, microorganisms are dividing, increasing in numbers such as doubling. If growth is not limited, doubling will continue at a constant rate so both the number of cells and the rate of population increase doubles with each consecutive time period. For this type of exponential growth, plotting the natural logarithm of cell number against time produces a straight line. The slope of this line is the specific growth rate of the microorganism, which is a measure of the number of divisions per cell per unit time. The actual rate of this growth (i.e. the slope of the line in the figure) depends upon the growth conditions, which affect the frequency of cell division events and the probability of both daughter cells surviving. When the media is depleted of nutrients and enriched with wastes, exponential growth cannot continue. During stationary phase, growth rate and death rate are equal or similar, which is shown as horizontal linear part of the growth curve. Without wishing to be bound by theory, this may be due to growth-limiting factor such as the depletion of an essential nutrient, and/or the formation of an inhibitory product such as an organic acid. At death phase the microorganism dies due to, for example, lack of nutrients, pH above or below the tolerance band for the microorganism, or other adverse conditions.

A microorganism culture is grown in a source culture media, by which a recycled culture media is obtained when the culture is the source culture is in a lag phase, an exponential phase, a stationary phase, or a death phase. In an embodiment, the recycled culture media is obtained by separating the recycled culture media from a source culture media, wherein the source culture media is in a lag phase, an exponential phase, a stationary phase, or a death phase. In another embodiment, the recycled culture media is obtained by separating the recycled culture media from a source culture media, wherein the source culture media is in a lag phase. In another embodiment, the recycled culture media is obtained by separating the recycled culture media from a source culture media, wherein the source culture media is in an exponential phase. In another embodiment, the recycled culture media is obtained by separating the recycled culture media from a source culture media, wherein the source culture media is in a stationary phase. In another embodiment, the recycled culture media is obtained by separating the recycled culture media from a source culture media, wherein the source culture media is in a stationary phase. In another embodiment, the recycled culture media is obtained by separating the recycled culture media from a source culture media, wherein the source culture media is in a death phase.

Cells and/or products produced by the methods of culturing microorganism described herein are collected at lag, exponential, stationary, or death phase. In an embodiment, cells and/or products produced are harvested or collected at lag, exponential, stationary, or death phase. In another embodiment, cells and/or products produced are harvested or collected at lag phase. In another embodiment, cells and/or products produced are harvested or collected at log phase. In another embodiment, cells and/or products produced are harvested or collected at lag phase. In another embodiment, cells and/or products produced are harvested or collected at stationary phase. In another embodiment, cells and/or products produced are harvested or collected at death phase.

When a microorganism culture reaches stationary phase, the concentration of the microorganisms in a culture reaches saturation. Saturation is determined by a number of measurements, including optical density, wet cell weight, dry cell weight, cell numbers, and/or time.

In an embodiment, the microorganism is grown to saturation measured as optical density at about 600 nm, wet cell weight, dry cell weight, or cell number. In an embodiment, saturation as measured by the optical density is about 2 to about 10. In an embodiment, saturation as measured by the wet cell weight is about 10 g/L to about 100 g/L. In an embodiment, saturation as measured by the dry cell weight is about 2 g/L to about 50 g/L. In an embodiment, saturation as measured by the cell number is about 2×10⁶ to about 100×10⁶ cells/mL. In an embodiment, the microorganism is grown for about 4 hours to about 350 hours, or up to about 75 days.

In general, feeding cell cultures can be categorized into four culturing styles: batch, fed-batch, semi-continuous and continuous culture. In batch culturing, a large volume of nutrients (media) is added to a population of cells. The cells are then grown until the inputs in the media are depleted, the desired concentration of cells is reached, or the desired product is produced. At this point the cells are harvested and the process is repeated. In fed-batch culturing, media is added either at a constant rate or components are added in as needed to maintain the cell population. Once it has reached a maximum or product formation is reached, the majority of the cells are harvested and the remaining cells are then used to start the next cycle. Fed-batch is when growth fermenter is not yet full, the media is fed in to bring the culture to a target density. Once full, and at target density, continuous harvesting begins, the goal of which is maintaining a full, target density culture. During semi-continuous culture, a sample of fixed volume is removed at regular time intervals to make measurements and/or harvest culture components, and an equal volume of fresh media is immediately added to the culture, thereby instantaneously enhancing nutrient concentrations and diluting cell concentration. In a continuous culture, the cells are cultured in media under conditions in which additions to and removals from the media can be made over an extended period of time. As such, nutrients, growth factors and space are not exhausted.

In an embodiment, the method of heterotrophically culturing a microorganism is batch, fed-batch, semi-continuous or continuous. In another embodiment, the method of heterotrophically culturing a microorganism is batch. In another embodiment, the method of heterotrophically culturing a microorganism is fed-batch. In another embodiment, the method of heterotrophically culturing a microorganism is semi-continuous. In another embodiment, the method of heterotrophically culturing a microorganism is continuous.

In semi-continuous and continuous culture, media is removed from the culture. The media can be removed at lag, exponential or stationary phase. In an embodiment, media is removed from the culture at lag, exponential or stationary phase. In another embodiment, media is removed from the culture at lag phase. In another embodiment, media is removed from the culture at exponential phase. In another embodiment, media is removed from the culture at stationary phase.

In semi-continuous and continuous culture, media can also be removed from the culture based on time interval. In an embodiment, the media is removed at about, or at least 1, 2, 3, 4, 5, 6, 7, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188 or 192 h from the beginning of the culture, or cycle of culture, or from a prior media addition. In semi-continuous and continuous culture, a cycle is defined as the turnover of the vessel (e.g., tank or bioreactor). A tank is a vessel or a system that supports the growth of a microorganism. Different parameters for growth are monitored and controlled for in the tank. These include the temperature, pH, oxygenation level and agitation. A tank can be 100 L an up to 20,000 L. Larger tanks are also possible such as 100,000 L or more. In an embodiment, the tank is at least 100 L, 1,000 L, 10,000 L, or 100,000 L. In another embodiment, the tank is up to 10,000 L, 100,000 L, 200,000 L, 500,000 L, or 1,000,000 L. A bioreactor is a vessel or a system that supports the growth of a microorganism. Different parameters for growth are monitored and controlled for in the bioreactor. These include the temperature, pH, oxygenation level and agitation. The scale of a bioreactor is smaller than a tank, such as 3 L-8 L, as well as larger bioreactors such as 36 L. A turnover is defined as the emptying of a vessel of one liquid such as a first media and the filling of the vessel by a second liquid such as a second media. With each subsequent emptying and filling that would represent another turnover. For example, a turnover of 2, turning over twice, or turns over 2 times would indicate that the tank was emptied and filled twice. During continuous culturing, there is substantially equal removal and addition of source media. One turnover in continuous culturing would be when the volume of the vessel has been removed and replenished in vessel. In an embodiment, the method is semi-continuous or continuous culture in a tank or a bioreactor. In another embodiment, the method is semi-continuous or continuous culture in a tank up to 10,000 L, 100,000 L, 200,000 L, 500,000 L or 1,000,000 L. In another embodiment, the method is semi-continuous or continuous culture in a bioreactor up to 3 L, 5 L, 8 L, 10 L, 20 L, 30 L, 35 L, 36 L, 40 L, or 50 L. In another embodiment, the media turns over 1, 2, 3, or 4 times a day in a tank or a bioreactor. In another embodiment, the media turns over up to 300 times in 75 days. In another embodiment, the media turns over at least 75, 150, 225, or 300 times in 75 days. In another embodiment, the method is continuous culture in a tank or a bioreactor, and the microorganism is grown for up to about 75 days. In another embodiment, the method is continuous culture in a tank or a bioreactor, the microorganism is grown for up to about 75 days, and the media turns over 300 times. In a specific embodiment, the method is continuous culture in a tank, the microorganism Euglena gracilis is grown for up to about 75 days, the media turns over 300 times, wherein the media is culture media, recycled culture media or hybrid culture media.

In fed-batch, semi-continuous and continuous culture, media is added to the culture. The media can be added at lag, exponential or stationary phase. In an embodiment, media is added to the culture at lag, exponential or stationary phase. In another embodiment, media is added to the culture at lag phase. In another embodiment, media is added to the culture at exponential phase. In another embodiment, media is added to the culture at stationary phase.

In fed-batch, semi-continuous and continuous culture, media can also be added to the culture based on time interval. In an embodiment, the media is added at about, or at least 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188 or 192 h from beginning of the culture, or cycle of culture, or from a prior media removal. In another embodiment, the media is added at about, or at most 10 min, 15 min, 30 min, 45 min, 60 min, 90 min, 2 h, 3 h, 4 h, 5 h, 6 h, 7 h, or 8 h from beginning of the culture, or cycle of culture, or from a prior media removal. In another embodiment, the media is added at approximately the same as the media is removed by the culture.

Additional media can be culture media, feed media, recycled culture media, spent media, supplemented media, and combinations thereof. Culture media (also known as growth media) is a media with components needed in order to grow or culture the cells. It could also be known as growth media. Feed media is a media with components that is added to a culture in order to replenish nutrients. Feed media is at a working concentration or a concentrated level of components to limit dilution of the culture. Feed media is a media with components that is added to a culture in order to replenish nutrients. Spent media is a media that has been used for cell culture i.e. culture media that has a lower level of growth components in it then at the start of culturing. A spent media is also determined by the content of carbohydrate in the media after being used for culturing cells. The carbohydrate can be a glucose, fructose, galactose, lactose, maltose, sucrose, molasses, glycerol, xylose, dextrose, honey, corn syrup. The spent media can contain less than about 50, 40, 30, 20, 15, 10, 8, 7, 6, 5, 4, 3, 2.5, 2, 1.5, 1, 0.5, 0.4, 0.3, 0.2, 0.1, or 0.01 g/L glucose, fructose, galactose, lactose, maltose, sucrose, molasses, glycerol, xylose, dextrose, honey, corn syrup, or combinations thereof. In an embodiment, the spent media comprises total carbohydrate of less than about 50, 40, 30, 20, 15, 10, 8, 7, 6, 5, 4, 3, 2.5, 2, 1.5, 1, 0.5, 0.4, 0.3, 0.2, 0.1, or 0.01 g/L. In an embodiment, the carbohydrate is glucose, fructose, galactose, lactose, maltose, sucrose, molasses, glycerol, xylose, dextrose, honey, corn syrup. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L glucose, fructose, galactose, lactose, maltose, sucrose, molasses, glycerol, xylose, dextrose, honey, corn syrup. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L glucose, fructose, galactose, sucrose, maltose and/or lactose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L glucose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L fructose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L galactose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L sucrose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L maltose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L lactose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L glycerol. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L xylose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L dextrose. In another embodiment, the spent media comprises less than about 50, 40, 30, 20, 15, 10, 8, 7, 6, 5, 4, 3, 2.5, 2, 1.5, 1, 0.5, 0.4, 0.3, 0.2, 0.1, or 0.01 g/L molasses. In another embodiment, the spent media comprises less than about 3, 2.5, 2, 1.5, 1, 0.5, 0.4, 0.3, 0.2, 0.1, or 0.01 g/L fructose, glucose, sucrose, or combinations thereof. In a specific embodiment, the spent media comprises less than about 3 g/L glucose.

The depletion of carbohydrate in the spent media can be expressed as a percentage of starting amount of carbohydrate at the beginning of a culture, or a culture cycle. In an embodiment, the spent media comprises total carbohydrate of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from amount at the beginning of culturing, or cycle of culturing. In another embodiment, the spent media comprises glucose, fructose, galactose, lactose, maltose, sucrose, molasses, glycerol, xylose, dextrose, honey, corn syrup, or combinations thereof, of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from amount at the beginning of culturing, or cycle of culturing. In another embodiment, the spent media comprises glucose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from amount at the beginning of culturing, or cycle of culturing. In another embodiment, the spent media comprises fructose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from amount at the beginning of culturing, or cycle of culturing. In another embodiment, the spent media comprises molasses of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from amount at the beginning of culturing, or cycle of culturing. In an embodiment, the spent media comprises galactose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from the amount at the beginning of culturing, or cycle of culturing. In another embodiment, the spent media comprises fructose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from the amount at the beginning of culturing, or cycle of culturing. In another embodiment, the spent media comprises sucrose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from the amount at the beginning of culturing, or cycle of culturing. In another embodiment, the spent media comprises maltose of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001% from the amount at the beginning of culturing, or cycle of culturing.

In addition to carbohydrate, carboxylic acid (also referenced herein as organic acids) is another carbon that is utilized by microorganism. Organic acids described herein may be in either protonated or de-protonated form. Useful carboxylic acid includes citric acid, citrate, fumaric acid, fumarate, malic acid, malate, pyruvic acid, pyruvate, succinic acid, succinate, acetic acid, acetate, lactic acid, and lactate. In an embodiment, the carboxylic acid is selected from the group consisting of citric acid, citrate, fumaric acid, fumarate, malic acid, malate, pyruvic acid, pyruvate, succinic acid, succinate, acetic acid, acetate, lactic acid, lactate, and combinations thereof. In another embodiment, the spent media, recycled culture media, or hybrid culture media comprises carboxylic acid of less than about 20, 10, 5, 4, 3, 2, 1, 0.5, 0.4, 0.3, 0.2, 0.1, 0.01, 0.001, 0.0005, 0.0001, 0.00005, 0.00001, or 0.000005 g/L. In another embodiment, the spent media, recycled culture media, or hybrid culture media comprises citric acid, citrate, fumaric acid, fumarate, malic acid, malate, pyruvic acid, pyruvate, succinic acid, succinate, acetic acid, acetate, lactic acid, lactate, and combinations thereof, of less than about 20, 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.01, 0.001, 0.0005, 0.0001, 0.00005, 0.00001, or 0.000005, g/L. In another embodiment, the spent media, recycled culture media, or hybrid culture media comprises citric acid of less than about 40, 30, 20, 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 g/L. In another embodiment, the spent media, recycled culture media, or hybrid culture media comprises acetate of less than about 10, 5, 4, 3, 2, 1, 0.5, 0.4, 0.3, 0.2, 0.1, 0.09, 0.08, 0.07, 0.06, 0.05, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.01, 0.001, 0.0005, 0.0001, 0.00005, 0.00001, or 0.000005 g/L. In another embodiment, the spent media, recycled culture media, or hybrid culture media comprises succinate of less than about 1, 0.5, 0.4, 0.3, 0.2, 0.1, 0.09, 0.08, 0.07, 0.06, 0.05, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 g/L. In another embodiment, the spent media, recycled culture media, or hybrid culture media comprises lactic acid of less than about 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.01, 0.001, 0.0005, 0.0001, 0.00005, 0.00001, or 0.000005 g/L. In another embodiment, the spent media, recycled culture media, or hybrid culture media comprises pyruvic acid of less than about 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 g/L. In another embodiment, the spent media, recycled culture media, or hybrid culture media comprises malate of less than about 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 g/L. In another embodiment, the spent media, recycled culture media, or hybrid culture media comprises fumaric acid of less than about 10, 5, 4, 3, 2, 1, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 g/L.

Recycled culture media is spent media that is used to culture cells for another passage, cycle, or for culturing cells from a different culture, lot, or strains. Recycled culture media is obtained by separating the recycled culture media from a source culture media, wherein the source culture media is in a lag phase, an exponential phase, or a stationary phase. Recycled culture media could be solely spent media, or it could be mixed with culture media (fresh growth media), or supplemented with one or more components that are depleted in the spent media. Recycled culture media can be obtained by separating the recycled culture media from a source culture media, wherein the source culture media is in a lag phase, an exponential phase, or a stationary phase. In an embodiment, recycled culture media is obtained by separating the recycled culture media from a source culture media, wherein the source culture media is in a lag phase, an exponential phase, or a stationary phase.

A hybrid culture media (also referred to herein as hybrid media or recycled hybrid media) is a culture media that contains an amount of recycled culture media (for example, a mixture of fresh media and recycled culture media). The hybrid culture media contains one or more carbohydrates, alcohols, lipids, proteins, yeast extract, amino acids and/or carboxylic acids. The carbohydrate in a hybrid culture is glucose, fructose, galactose, lactose, maltose, sucrose, molasses, glycerol, xylose, dextrose, honey, corn syrup, or combinations thereof. In an embodiment, the hybrid culture media comprise one or more carbohydrates, alcohols, lipids, proteins, yeast extract, amino acids and/or carboxylic acids. In an embodiment, the carbohydrate is glucose, fructose, galactose, lactose, maltose, sucrose, molasses, glycerol, xylose, dextrose, honey, corn syrup, or combinations thereof. In another embodiment, the hybrid culture media comprises fresh media and recycled culture media. In another embodiment, the hybrid culture media comprises about 10%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% recycled or spent media. The hybrid culture media also contains growth hormones, such as cytokinins. In an embodiment, the hybrid culture media comprises about 0.01 pmol/mL to about 100 nmol/mL of cytokinins. All concentrations of cytokinins described herein expressed in nmol/mL and pmol/mL also includes the numeric value in nmol/g and pmol/g, respectively. In an embodiment, the hybrid culture media comprises about 0.01 pmol/mL to about 0.25 nmol/mL cytokinins, about 0.01 pmol/mL to about 0.50 nmol/mL cytokinins, about 0.01 pmol/mL to about 0.75 nmol/mL cytokinins, about 0.01 pmol/mL to about 1 nmol/mL cytokinins, about 0.01 pmol/mL to about 1.5 nmol/mL cytokinins, about 0.01 pmol/mL to about 2 nmol/mL cytokinins, about 0.01 pmol/mL to about 30 nmol/mL cytokinins, about 0.01 pmol/mL to about 50 nmol/mL cytokinins about 0.05 nmol/mL to about 100 nmol/mL, about 0.05 nmol/mL to about 75 nmol/mL, about 0.05 nmol/mL to about 50 nmol/mL, about 0.05 nmol/mL to about 40 nmol/mL, about 0.05 nmol/mL to about 35 nmol/mL, about 0.05 nmol/mL to about 30 nmol/mL, about 0.05 nmol/mL to about 25 nmol/mL, about 0.05 nmol/mL to about 20 nmol/mL, about 0.05 nmol/mL to about 15 nmol/mL, about 0.05 nmol/mL to about 10 nmol/mL, about 0.05 nmol/mL to about 5 nmol/mL, about 0.05 nmol/mL to about 4 nmol/mL, about 0.05 nmol/mL to about 3 nmol/mL, about 0.05 nmol/mL to about 2.5 nmol/mL, about 0.05 nmol/mL to about 2 nmol/mL, about 0.05 nmol/mL to about 1.5 nmol/mL, about 0.05 nmol/mL to about 1 nmol/mL, about 0.05 nmol/mL to about 0.5 nmol/mL, about 0.05 nmol/mL to about 0.1 nmol/mL of one or more cytokinins. In some embodiments, the hybrid culture media comprises at least about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, about 0.4 nmol/mL, about 0.3 nmol/mL, about 0.2 nmol/mL, about 0.1 nmol/mL, about 0.09 nmol/mL, about 0.08 nmol/mL, about 0.07 nmol/mL, about 0.06 nmol/mL, about 0.05 nmol/mL, about 0.04 nmol/mL, about 0.03 nmol/mL, about 0.02 nmol/mL, about 0.01 nmol/mL, about 0.009 nmol/mL of one or more cytokinins. The concentration of cytokinins described herein may be with reference to total cytokinin levels, total levels of groups of cytokinins (e.g., Free Bases (FBs), Ribosides (RBs), Nucleotides (NTs), Glucosides (GLUCs), and Methylthiols (METs)), total levels of one or more types of cytokinins (e.g., Dihydrozeatin (DZ), trans-zeatin (tZ), Isopentenyl adenine (iP), and cis-zeatin (cZ)), one or more individual cytokinins (e.g., one or more of DZR, tZR, cZR, iPR, DZNT, tZNT, cZNT, iPNT, 2MeSZ, 2MeSiP, 2MeSZR, 2MeSiPA, DZOG, DZROG, cZROG, DZ9G, tZOG, cZOG, tZ9G), or any combination thereof (see Tables 1-5). In some embodiments, the hybrid culture media comprises less than about 0.01 nmol/mL of one or more cytokinins. In some embodiments, the hybrid culture media is free or substantially free of one or more cytokinins (e.g., DZR, tZR, cZR, iPR, DZNT, tZNT, cZNT, iPNT, 2MeSZ, 2MeSiP, 2MeSZR, 2MeSiPA, DZOG, DZROG, cZROG, DZ9G, tZOG, cZOG, tZ9G).

In some embodiments, the hybrid culture media comprises abscisic acid (ABA). In some embodiments, the hybrid culture media comprises about 0.01 pmol/mL to about 100 nmol/mL of ABA. In some embodiments, the hybrid culture media comprises about 0.001 nmol/mL to about 100 nmol/mL, about 0.001 nmol/mL to about 75 nmol/mL, about 0.001 nmol/mL to about 50 nmol/mL, about 0.001 nmol/mL to about 40 nmol/mL, about 0.001 nmol/mL to about 35 nmol/mL, about 0.001 nmol/mL to about 30 nmol/mL, about 0.001 nmol/mL to about 25 nmol/mL, about 0.001 nmol/mL to about 20 nmol/mL, about 0.001 nmol/mL to about 15 nmol/mL, about 0.001 nmol/mL to about 10 nmol/mL, about 0.001 nmol/mL to about 5 nmol/mL, about 0.001 nmol/mL to about 4 nmol/mL, about 0.001 nmol/mL to about 3 nmol/mL, about 0.001 nmol/mL to about 2.5 nmol/mL, about 0.001 nmol/mL to about 2 nmol/mL, about 0.001 nmol/mL to about 1.5 nmol/mL, about 0.001 nmol/mL to about 1 nmol/mL, about 0.001 nmol/mL to about 0.5 nmol/mL, or about 0.001 nmol/mL to about 0.1 nmol/mL of ABA. In some embodiments, the hybrid culture media comprises at least about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, about 0.4 nmol/mL, about 0.3 nmol/mL, about 0.2 nmol/mL, about 0.1 nmol/mL, about 0.09 nmol/mL, about 0.08 nmol/mL, about 0.07 nmol/mL, about 0.06 nmol/mL, about 0.05 nmol/mL, about 0.04 nmol/mL, about 0.03 nmol/mL, about 0.02 nmol/mL, about 0.01 nmol/mL, or about 0.009 nmol/mL of ABA (see Tables 2-3). In some embodiments, the hybrid culture media comprises less than about 0.001 nmol/mL of ABA. In some embodiments, the hybrid culture media is free or substantially free of ABA.

Supplemented media is a media that is culture, feed, spent or recycled culture media that has further been supplemented with one or more additional component to increase growth, health and/or production of the cells. In an embodiment, the additional media is selected from the group consisting of a culture media, a feed media, a recycled culture media, a spent media, a supplemented media, and combinations thereof, optionally a recycled culture media, or a supplemented media. In another embodiment, the additional media is a culture media. In an embodiment, the recycled culture media is selected from the group consisting of a culture media, a feed media, a spent media, a supplemented media, and combinations thereof, optionally a spent media, or a supplemented media. In another embodiment, the additional media is a feed media. In another embodiment, the additional media is a spent media. In another embodiment, the additional media is a recycled culture media. In another embodiment, the additional media is a supplemented media.

In some embodiments, any media as described herein is supplemented with one or more cytokinins and/or ABA. Supplementation with an amount of one or more cytokinins and/or ABA results in a total level of one or more cytokinins and/or ABA. If, for example, a level of the one or more cytokinins and/or ABA is already present in the media being supplemented prior to supplementation, the total level of the one or more cytokinins and/or ABA will be higher than the level of cytokinins and/or ABA with which the media is supplemented. In some embodiments, the media is supplemented with or to a total level of about 0.01 pmol/mL to about 100 nmol/mL. In some embodiments, the media is supplemented with or to a total level of about 0.05 nmol/mL to about 100 nmol/mL, about 0.05 nmol/mL to about 75 nmol/mL, about 0.05 nmol/mL to about 50 nmol/mL, about 0.05 nmol/mL to about 40 nmol/mL, about 0.05 nmol/mL to about 35 nmol/mL, about 0.05 nmol/mL to about 30 nmol/mL, about 0.05 nmol/mL to about 25 nmol/mL, about 0.05 nmol/mL to about 20 nmol/mL, about 0.05 nmol/mL to about 15 nmol/mL, about 0.05 nmol/mL to about 10 nmol/mL, about 0.05 nmol/mL to about 5 nmol/mL, about 0.05 nmol/mL to about 4 nmol/mL, about 0.05 nmol/mL to about 3 nmol/mL, about 0.05 nmol/mL to about 2.5 nmol/mL, about 0.05 nmol/mL to about 2 nmol/mL, about 0.05 nmol/mL to about 1.5 nmol/mL, about 0.05 nmol/mL to about 1 nmol/mL, about 0.05 nmol/mL to about 0.5 nmol/mL, or about 0.05 nmol/mL to about 0.1 nmol/mL. In some embodiments, any media as described herein is supplemented with or to a total level of at least about 100 nmol/mL, about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, or about 0.1 nmol/mL. In some embodiments, any media as described herein is supplemented with one or more cytokinins selected from DZR, tZR, cZR, iPR, DZNT, tZNT, cZNT, iPNT, 2MeSZ, 2MeSiP, 2MeSZR, 2MeSiPA, DZOG, DZROG, cZROG, DZ9G, tZOG, cZOG, and/or tZ9G and or any type or form (see Tables 1-5). Such media may be used to prime or pre-treat microalgae, e.g., Euglena, seed to, e.g., encourage quicker cell division.

The microorganism in the culture is harvested at lag, exponential, or stationary phase. In an embodiment, the microorganism is harvested at lag, exponential, or stationary phase.

The recycled culture media is also from the same culture, passage and/or cycle of a culture. In an embodiment, the harvested microorganisms are separated from the media and the media is recycled back into the culture. In another embodiment, the media recycled back into the culture is spent media.

Harvest of microorganisms in a culture is done in whole or in part. In an embodiment, harvested microorganisms in a culture are about 1, 2, 3, 4, 5, 10, 15, 20, 25, 30, 35, 40, 45, 50, 55, 60, 65, 70, 75, 80, 85, 90, 95 or 100% of total source culture. Harvest methods are known in the art, for example, harvesting by settling of cells where source media is left in a fermenter. In an embodiment, the harvesting of microorganisms is harvesting by settling cells. At scale, microorganisms are left to settle in the bottom of the tank to separate cells from source media. The microorganisms are then removed from the bottom of the tank and the remaining spent media is left in the tank. The tank is then supplemented with fresh growth media, recycled culture media, or hybrid culture media. Harvesting of microorganisms can also be established by physical methods, such as centrifugation. In this process, centrifugal force separates the cells from the source media. At scale, this could be accomplished by disk stack centrifuges. In an embodiment, the harvesting of microorganisms is harvesting by disk stack centrifuges.

The present disclosure describes useful percentages of spent media or recycled culture media in a hybrid culture media that also contains a fresh media. When describing hybrid culture media, a 10% hybrid culture media contains 10% spent or recycled culture media and 90% fresh media. Useful percentages of spent or recycled culture media in a hybrid culture media are about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99%, about 10% to about 99.99%, optionally about 10% to about 75% of the spent or recycled culture media, optionally about 25% to about 75%, optionally about 50% to about 75%, optionally about 75%. In an embodiment, about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, or 100% of media, optionally spent or cycled media, is returned to the culture. In another embodiment, about 10% to about 99.99% spent or recycled culture media is returned to the culture, optionally 10% to about 75%, optionally about 25% to about 75%, optional about 50% to about 75%, optionally about 75%. In another embodiment, about 25% of the spent or recycled culture media is returned to the culture. In another embodiment, about 50% of the spent or recycled culture media is returned to the culture. In another embodiment, about 75% of the spent or recycled culture media is returned to the culture.

The pH of a media affects growth of a microorganism in culture. The person skilled in the art can readily modify the pH of a growth media with organic acids, such as nitric acid, hydrochloric acid, sulphuric acid, and citric acid, or bases, such as sodium hydroxide, sodium carbonate, and sodium bicarbonate. The pH of the media is between about 2 to about 8, about 2.5 to about 5, about 2.5 to about 4, about 2.5 to about 3.5. In an embodiment, the culture media is maintained at a pH of between about 2 to about 8, optionally about 2.5 to about 5, optionally between about 2.5 to about 4.

The term “cycle” as used herein refers to a set period of time for cell growth. For example, a cycle is for culturing cells for about 2, 3, 4, or 5 days, or for about 48, 54, 60, 66, 72, 78, 84, 90, 96, 102, 108, 114, or 120 hours. In an embodiment, a cycle is for culturing a microorganism for about 2, 3, 4, or 5 days, or for about 48, 54, 60, 66, 72, 78, 84, 90, 96, 102, 108, 114, or 120 hours. In another embodiment, a cycle is for continuous culturing a microorganism for about, or at least 2, 3, 4, 5, 6, 7, 8, 9, 10, 11, or 12 hours.

The term “consecutive cycles” as used herein refers to cycles of growth that sequentially follow each other. For example, in experiments described herein where cells were grown for 3 consecutive cycles, at the end of the 1st growth cycle and using 3 days as an example of the period of time for a cycle, the culture was separated into cell biomass, and supernatant (spent growth media). A new cycle is started by inoculating 2 million cells/mL back into the flask that contains either fresh growth media or a percentage of recycled growth media. This begins Cycle 2 and the cells are grown for another period of time (end of Cycle 2, i.e. at the end of a second 3-day period). A Cycle 3 is started and completed the same way that Cycle 2 was set up. A culture of cells can undergo a number of cycles, for example, at least or about 2, 3, 4, 5, 6, 7, 8, 10, 11, 12, 13, 14, 15, 16, 17, 18, 19, 20, 25, 30, 35, 40, 45 or 50. In an embodiment, the microorganism is cultured for at least or about 2, 3, 4, 5, 6, 7, 8, 10, 11, 12, 13, 14, 15, 16, 17, 18, 19, 20, 25, 30, 35, 40, 45, 50, 60, 70, 80, 90, 100, 150, 200, 250, or 300 cycles.

The term “conversion efficiency” as used herein refers to a percentage of the biomass generated by the amount of solutes consumed by the microorganism in the source media used. When more biomass is generated with a fixed amount of media components, the conversion efficiency is higher. When less biomass is generated with a fixed amount of media components, the conversion efficiency is lower. As such, the higher “conversion efficiency” represents more conversion of solutes into biomass. In an embodiment, the conversion efficiency of cells in a media, optionally hybrid culture media, recycled culture media or supplemented media, is at least or about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or at 100% (weight biomass/weight solutes). Conversion efficiency of a hybrid culture media or a supplemented media can also be compared with the corresponding fresh media control containing about the same amount of carbon and/or carbohydrate content. This “relative conversion efficiency” is the conversion rate of a recycled culture media or a supplemented media divided by the conversion rate of the corresponding media. The conversion efficiency of a hybrid culture media or a supplemented media is near as well or better than the corresponding fresh media. The relative conversion efficiency of a hybrid culture media or a supplemented media is at least or about 85%, 90%, 91%, 92%, 93%, 94%, 95%, 96%, 97%, 98%, 99%, 100%, 101%, 102%, 103%, 104%, 105%, 106%, 107%, 108%, 109%, 110%, 115%, 120%, or 125%. In an embodiment, the relative conversion efficiency of a hybrid culture media or a supplemented media is about 10, 15, 20, 30, 40, 50, or 60% to about 70, 75, 85, 90, 95, 100, 105, 110, 115, 120, or 125%. In an embodiment, the relative conversion efficiency of a hybrid culture media or a supplemented media is about 40% to about 70%. In an embodiment, the relative conversion efficiency of a hybrid culture media or a supplemented media is about 85% to about 100%. In another embodiment, the relative conversion efficiency of a hybrid culture media or a supplemented media is at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 101%, 102%, 103%, 104%, 105%, 106%, 107%, 108%, 109%, 110%, 115%, 120%, or 125%.

As used in this disclosure, the term “microorganism” and its derivatives, as used herein, include heterotrophic or mixotrophic microorganisms that are prokaryotes or eukaryotes. In an embodiment, the microorganism is a Euglena sp., a Chlorella sp. or a Schizochytrium sp. In another embodiment, the microorganism species is a Chlorella sp. In another embodiment, the microorganism species is a Schizochytrium sp. In another embodiment, the microorganism species is a Euglena sp. In another embodiment, the microorganism is selected from the group consisting of Euglena gracilis, Euglena sanguinea, Euglena deses, Euglena mutabilis, Euglena acus, Euglena virdis, Euglena anabaena, Euglena geniculata, Euglena oxyuris, Euglena proxima, Euglena tripteris, Euglena chlamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena variabilis, Euglena caudata, Euglena minima, Euglena communis, Euglena magnifica, Euglena terricola, Euglena velata, Euglena repulsans, Euglena clavata, Euglena lata, Euglena tuberculata, Euglena contabrica, Euglena ascusformis, Euglena ostendensis, Chlorella autotrophica, Chlorella colonials, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, Chlorella vulgaris, Schizochytrium aggregatum, Schizochytrium limacinum, Schizochytrium minutum and combinations thereof. In a specific embodiment, the Euglena sp. is Euglena gracilis.

Microorganism such as Euglena gracilis is capable of heterotrophic and phototrophic culturing. The methods described herein do not involve phototrophic culturing. In an embodiment, the method does not comprise phototrophic culturing.

In using recycled culture media for culturing microorganism, a cleaning or sterilization is typically involved. The step is for degrading toxic chemicals that negatively affect cells growth. The methods described herein do not involve a cleaning or sterilization step, for example, autoclaving, filtration, UV or heat sterilizing of the media, for example, recycled, spent or supplemented media. In an embodiment, the method does not comprise hybrid media sterilization. In another embodiment, the method does not comprise autoclaving, filtration, UV treatment or heat sterilizing of the media. In another embodiment, the method does not comprise autoclaving of the media. In another embodiment, the method does not comprise filtration of the media. In another embodiment, the method does not comprise UV treatment of the media. In another embodiment, the method does not comprise heat sterilization of the media.

The present disclosure also describes production of different types of cytokinins, a hormone implicated in growth regulation of microorganisms. Cytokinins can be derived from an exogenous source such as growth media and/or are produced by culturing Euglena using fresh media, spent media, recycled culture media, or hybrid culture media. Cytokinins are in the forms of free bases, ribosides, nucleotides, methylthiols, glucosides and aromatic. In some embodiments, cytokinins include one or more of those cytokinins shown in Table 1. In an embodiment, the method described herein comprises culturing a microorganism, wherein the microorganism is Euglena, wherein the method of culturing Euglena produces about 0.01 pmol/g or mL to about 100 nmol/g or mL cytokinins. In some embodiments, the method described herein comprises culturing a microorganism, wherein the microorganism is Euglena, wherein the method of culturing Euglena produces about 0.05 nmol/mL to about 100 nmol/mL, about 0.05 nmol/mL to about 75 nmol/mL, about 0.05 nmol/mL to about 50 nmol/mL, about 0.05 nmol/mL to about 40 nmol/mL, about 0.05 nmol/mL to about 35 nmol/mL, about 0.05 nmol/mL to about 30 nmol/mL, about 0.05 nmol/mL to about 25 nmol/mL, about 0.05 nmol/mL to about 20 nmol/mL, about 0.05 nmol/mL to about 15 nmol/mL, about 0.05 nmol/mL to about 10 nmol/mL, about 0.05 nmol/mL to about 5 nmol/mL, about 0.05 nmol/mL to about 4 nmol/mL, about 0.05 nmol/mL to about 3 nmol/mL, about 0.05 nmol/mL to about 2.5 nmol/mL, about 0.05 nmol/mL to about 2 nmol/mL, about 0.05 nmol/mL to about 1.5 nmol/mL, about 0.05 nmol/mL to about 1 nmol/mL, about 0.05 nmol/mL to about 0.5 nmol/mL, about 0.05 nmol/mL to about 0.1 nmol/mL of one or more cytokinins. In some embodiments, the hybrid culture media comprises at least about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, about 0.4 nmol/mL, about 0.3 nmol/mL, about 0.2 nmol/mL, about 0.1 nmol/mL, about 0.09 nmol/mL, about 0.08 nmol/mL, about 0.07 nmol/mL, about 0.06 nmol/mL, about 0.05 nmol/mL, about 0.04 nmol/mL, about 0.03 nmol/mL, about 0.02 nmol/mL, about 0.01 nmol/mL, about 0.009 nmol/mL of one or more cytokinins. The concentration of cytokinins described herein may be with reference to total cytokinin levels, total levels of groups of cytokinins (e.g., Free Bases (FBs), Ribosides (RBs), Nucleotides (NTs), Glucosides (GLUCs), and Methylthiols (METs)), total levels of one or more types of cytokinins (e.g., Dihydrozeatin (DZ), trans-zeatin (tZ), Isopentenyl adenine (iP), and cis-zeatin (cZ)), one or more individual cytokinins (e.g., one or more of DZR, tZR, cZR, iPR, DZNT, tZNT, cZNT, iPNT, 2MeSZ, 2MeSiP, 2MeSZR, 2MeSiPA, DZOG, DZROG, cZROG, DZ9G, tZOG, cZOG, tZ9G), or any combination thereof (see Tables 1-5). Accordingly, in one embodiment, the cytokinin is in the form selected from the group consisting of free bases, ribosides, nucleotides, methylthiols and glucosides.

Abscisic acid (ABA) is a hormone which plays a role in growth and metabolite production in microorganisms. In an embodiment, the method described herein comprises culturing a microorganism wherein the microorganism is Euglena, wherein the method of culturing Euglena produces about 0.01 pmol/g or mL to 100 nmol/g or mL of ABA. All concentrations of ABA described herein expressed in nmol/mL and pmol/mL also includes the numeric value in nmol/g and pmol/g, respectively. In an embodiment, the method described herein comprises culturing a microorganism wherein the microorganism is Euglena, wherein the method of culturing Euglena produces about 0.001 nmol/mL to about 100 nmol/mL, about 0.001 nmol/mL to about 75 nmol/mL, about 0.001 nmol/mL to about 50 nmol/mL, about 0.001 nmol/mL to about 40 nmol/mL, about 0.001 nmol/mL to about 35 nmol/mL, about 0.001 nmol/mL to about 30 nmol/mL, about 0.001 nmol/mL to about 25 nmol/mL, about 0.001 nmol/mL to about 20 nmol/mL, about 0.001 nmol/mL to about 15 nmol/mL, about 0.001 nmol/mL to about 10 nmol/mL, about 0.001 nmol/mL to about 5 nmol/mL, about 0.001 nmol/mL to about 4 nmol/mL, about 0.001 nmol/mL to about 3 nmol/mL, about 0.001 nmol/mL to about 2.5 nmol/mL, about 0.001 nmol/mL to about 2 nmol/mL, about 0.001 nmol/mL to about 1.5 nmol/mL, about 0.001 nmol/mL to about 1 nmol/mL, about 0.001 nmol/mL to about 0.5 nmol/mL, or about 0.001 nmol/mL to about 0.1 nmol/mL of ABA. In some embodiments, the hybrid culture media comprises at least about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, about 0.4 nmol/mL, about 0.3 nmol/mL, about 0.2 nmol/mL, about 0.1 nmol/mL, about 0.09 nmol/mL, about 0.08 nmol/mL, about 0.07 nmol/mL, about 0.06 nmol/mL, about 0.05 nmol/mL, about 0.04 nmol/mL, about 0.03 nmol/mL, about 0.02 nmol/mL, about 0.01 nmol/mL, or about 0.009 nmol/mL of ABA (see Tables 2-3). In some embodiments, the method described herein comprises culturing a microorganism wherein the microorganism is Euglena, wherein the method of culturing Euglena produces less than about 0.001 nmol/mL of ABA.

In a specific embodiment, the method of culturing Euglena gracilis microorganism comprises:
culturing the microorganism in a hybrid culture media;
maintaining the microorganism heterotrophically in an environment entirely free from light;
maintaining a pH of between about 2.5 to about 4;
wherein the microorganism is inoculated at about 1×10⁵ cells/mL to about 5×10⁷ cells/mL;
wherein the hybrid culture media comprises a carbohydrate;
wherein the carbohydrate is glucose, fructose, galactose, lactose, maltose, sucrose, molasses, glycerol, xylose, dextrose, honey, and/or corn syrup;
wherein the hybrid culture media comprises fresh media and recycled culture media;
wherein the recycled culture media is obtained by separating the recycled culture media from a source culture media, wherein the source culture media is in a lag phase, an exponential phase, or a stationary phase;
wherein the method does not comprise hybrid media sterilization; and
wherein the culture media maintains a relative conversion efficiency of at least 85%, 90%, 91%, 92%, 93%, 94%, 95%, 96%, 97%, 98%, 99% or 100%.

Also provided is a method for producing a recycled culture media suitable for heterotrophically culturing a Euglena sp. microorganism, Schizochytrium sp. microorganism, or a Chlorella sp microorganism, comprising:
culturing the microorganism in a culture media comprising a carbohydrate; maintaining an environment with substantially, or entirely no light;
producing recycled culture media;
separating recycled culture media from cells; and
collecting recycled culture media;
wherein the microorganism is cultured until the carbohydrate is below 3 g/L;
wherein the microorganism is Euglena gracilis;
wherein the recycled culture media is obtained by separating the recycled culture media from a source culture media;
wherein the source culture media is in a lag phase, an exponential phase, or a stationary phase; and
wherein the source culture media is a hybrid culture media or a stock culture media.

In an embodiment, the recycled culture media comprises total carbohydrate of less than about 50, 40, 30, 25, 20, 15, 10, 9, 8, 7, 6, 5, 4, 3, 2.5, 2, 1.5, 1, 0.5, 0.4, 0.3, 0.2, 0.1, or 0.01 g/L. In another embodiment, the carbohydrate is glucose, fructose, galactose, lactose, maltose, sucrose, molasses, glycerol, xylose, dextrose, honey, corn syrup, or combinations thereof.

The recycled culture media is mixed with a fresh media to produce a hybrid culture media by the methods described herein. Accordingly, the methods provided herein also produce a hybrid culture media. In an embodiment, a hybrid culture media is produced by mixing a fresh media with a recycled culture media produced by a method described herein. In an embodiment, a hybrid culture media comprises about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% recycled culture media or spent media.

In another aspect, the method for producing spent or recycled culture media suitable for heterotrophically culturing a microorganism produces a spent media spent or recycled culture media comprising positive growth regulators. Positive growth regulators can be growth hormone, for example cytokinins, or any protein, carbohydrate, nucleotide, or lipid that promotes cell growth. These can be excreted from cells into the spent media, or exogenously added from another source.

Also provided is a use of the culture media, spent media, recycled culture media, supplemented media or hybrid culture media produced by the method described herein for culturing a microorganism. In an embodiment, the microorganism is a Euglena sp. In an embodiment, the Euglena sp. is Euglena gracilis.

The methods, uses and media disclosed herein can be applied to any microorganism. The methods, uses and media disclosed herein are scalable at higher capacity.

In an aspect, the methods described herein comprise culturing in fresh media, adding recycled media to form hybrid media, culturing in hybrid media, repeating the step of adding recycled media and culturing. Also provided herein are compositions comprising culture media, spent media, recycled culture media, supplemented media and/or hybrid culture media produced by the methods described herein.

Also provided herein are compositions comprising a fraction or extract of the culture media, spent media, recycled culture media, supplemented media or hybrid culture media produced by the method described herein. In some embodiments, such fractions comprise phytohormones, organic acids, amino acids, exopolysaccharides, extracellular lipids, and/or allelopathic chemicals extracted or separated from the culture media, spent media, recycled culture media, supplemented media or hybrid culture media produced by the method described herein. In some embodiments, such fractions comprise pure or substantially pure phytohormones, organic acids, amino acids, exopolysaccharides, extracellular lipids, and/or allelopathic chemicals. In some embodiments, the fraction comprises one or more cytokinins. Suitable cytokinins are described herein. In some embodiments, the fraction comprises ABA. Compositions comprising such fractions may be used to prime or pre-treat microalgae, e.g., Euglena, seed to, e.g., encourage quicker cell division.

Also provided in connection with any composition or method herein is removing inhibiting molecules (e.g., heavy metals, phenols, fatty acids, ammonia, and/or cell debris) from the composition or media.

Compositions may take any suitable form. For example, compositions described herein may be liquid. In some embodiments, a composition described herein is a solid composition (e.g., in the form of granules, pellets, or beads). Compositions described herein may be stable (e.g., able to withstand environmental pressure such as heat, freezing, freeze-thaw cycles, heating, pressure, oxidizing conditions, and combinations thereof without significant loss of activity or concentration of one or more composition components). In some embodiments, the composition is stable for about at least 1, 2, 3, 4, 5, 6, 7, 14, 30, 60, or 90 days. In some embodiments, the composition is heat stable. In some embodiments, organic acids in the composition is heat stable.

Microalgae secrete extracellular metabolites into medium during growth including, for example, phytohormones (e.g., cytokinins or ABA), organic acids (e.g., citric acid, citrate, fumaric acid, fumarate, malic acid, malate, pyruvic acid, pyruvate, succinic acid, succinate, acetic acid, acetate, lactic acid, and/or lactate), amino acids, exopolysaccharides, extracellular lipids, allelopathic chemicals, much of which is disposed of as waste following biomass harvesting. This spent media gives rise to environmental pollution. Large volumes of algae-free media (or substantially algae-free media) exist in, e.g., unexploited flow-through cultures and after biomass harvesting of batch cultures. Recycling methods described herein lead to generation of high value products from spent medium. Spent/cell free medium has the potential to yield high value metabolites either by targeted extraction of metabolites or reuse of the spent material for other purposes such as an agricultural fertilizer/growth promoter or soil conditioners.

Algae, including microalgae, blue-green algae and cyanobacteria have been used as fertilizer additives. Excretion of exopolysaccharides and bioactive substances by algae and cyanobacteria has a proven role in recovering soil nutrients and mobilization of insoluble forms of inorganic phosphates. Algal proteoglycans possess adhesive properties which can easily fasten cells to solid surfaces and aggregated soil particles impacts temperature, aeration, and infiltration rates of the soil. This can improve the physical environment for the crop. Mucopolysaccharides, carbohydrates and organic matter can improve soil fertility and its capacity to retain humidity. Microalgae (e.g., E. gracilis) cultured according to methods described herein has the capability to excrete these valuable metabolites into culture medium.

Accordingly, also provided herein are biofertilizer or soil conditioning compositions comprising culture media, spent media, recycled culture media, supplemented media and/or hybrid culture media (or fractions thereof) produced by the methods described herein.

Also provided herein is a use of the culture media, spent media, recycled culture media, supplemented media and/or hybrid culture media (or fractions thereof) produced by the methods described herein in compositions for use as a soil conditioner or biofertilizer. In some embodiments, such use includes contacting soil with an effective amount of a composition described herein. In some embodiments, the soil may be in the immediate vicinity of a planted seed or growing plant. In some embodiments, a composition described herein is a liquid composition and can be supplied to the soil by any suitable means including, but not limited to, injection (e.g., drip irrigation), spray, or by soil drench method where a liquid composition is poured on the soil. In some embodiments, a composition described herein is a solid composition (e.g., in the form of granules, pellets, or beads) and can be supplied to the soil by any suitable means.

In some embodiments, compositions described herein comprise microalgae cells. In some embodiments, compositions described herein are cell free or substantially cell free.

In some embodiments, any media as described herein is supplemented with one or more cytokinins and/or ABA. Supplementation with an amount of one or more cytokinins and/or ABA results in a total level of one or more cytokinins and/or ABA. If, for example, a level of the one or more cytokinins and/or ABA is already present in the media being supplemented prior to supplementation, the total level of the one or more cytokinins and/or ABA will be higher than the level of cytokinins and/or ABA with which the media is supplemented. In some embodiments, the media is supplemented with or to a total level of about 0.01 pmol/mL to about 100 nmol/mL. In some embodiments, the media is supplemented with or to a total level of about 0.05 nmol/mL to about 100 nmol/mL, about 0.05 nmol/mL to about 75 nmol/mL, about 0.05 nmol/mL to about 50 nmol/mL, about 0.05 nmol/mL to about 40 nmol/mL, about 0.05 nmol/mL to about 35 nmol/mL, about 0.05 nmol/mL to about 30 nmol/mL, about 0.05 nmol/mL to about 25 nmol/mL, about 0.05 nmol/mL to about 20 nmol/mL, about 0.05 nmol/mL to about 15 nmol/mL, about 0.05 nmol/mL to about 10 nmol/mL, about 0.05 nmol/mL to about 5 nmol/mL, about 0.05 nmol/mL to about 4 nmol/mL, about 0.05 nmol/mL to about 3 nmol/mL, about 0.05 nmol/mL to about 2.5 nmol/mL, about 0.05 nmol/mL to about 2 nmol/mL, about 0.05 nmol/mL to about 1.5 nmol/mL, about 0.05 nmol/mL to about 1 nmol/mL, about 0.05 nmol/mL to about 0.5 nmol/mL, or about 0.05 nmol/mL to about 0.1 nmol/mL. In some embodiments, any media as described herein is supplemented with or to a total level of at least about 75 nmol/mL, about 50 nmol/mL, about 35 nmol/mL, about 30 nmol/mL, about 25 nmol/mL, about 20 nmol/mL, about 15 nmol/mL, about 10 nmol/mL, about 5 nmol/mL, about 4 nmol/mL, about 3 nmol/mL, about 2.5 nmol/mL, about 2 nmol/mL, about 1.5 nmol/mL, about 1 nmol/mL, about 0.5 nmol/mL, or about 0.1 nmol/mL. In some embodiments, any media as described herein is supplemented with one or more cytokinins selected from DZR, tZR, cZR, iPR, DZNT, tZNT, cZNT, iPNT, 2MeSZ, 2MeSiP, 2MeSZR, 2MeSiPA, DZOG, DZROG, cZROG, DZ9G, tZOG, cZOG, and/or tZ9G (see Tables 1-5).

It will be appreciated by a person skilled in the art that embodiments of the uses of the present disclosure can be varied as described herein for the methods of the present disclosure.

The following non-limiting examples are illustrative of the present application:

### EXAMPLES

### Example 1: Hybrid Culture Media A and Hormone Signaling

Introduction

Increasing the efficiency of growth and product yield is of interest not just to academia, but to industry as well. Without wishing to be bound by theory, it may be possible to manipulate and track parameters pertaining to essential growth. Work with microalgae suggested that cytokinins (CKs) act as growth regulators. Described herein is the use of recycled culture media and its influence on biomass accumulation, cell counts, pH, glucose consumption, supernatant depletion, cell counts and CKs in Euglena gracilis.

Methods and Materials

Preliminary Culture Growth Conditions

Euglena gracilis was grown batch-cultured heterotrophically in autoclaved a fresh media containing molasses (3 g/L), yeast extract (5 g/L), ammonium phosphate monobasic (2 g/L), vegetable oil (2 g/L), and ethanol (2 g/L). E. gracilis was cultured in a 2 L culture flask with ajar solid cap in an Adolf Kuhner AG (Model, ISF-4-V) incubator set at 100 min⁻¹ shaking speed at 29.0° C.

Experimental Growth Conditions

E. gracilis was harvested as a cell concentrate from the incubated preliminary culture and used to propagate experiments. Each series of experiments employed the same fresh media. Recycled supernatant was recirculated from the source culture in a 75% spent media to 25% fresh media ratio using a fed-batch technique (creating a hybrid culture media-75%).

### Experiment 1 & 2 Culture Conditions and Data Collection

A cell concentrate from the preliminary culture was used to inoculate 5×500 mL (4 test replicates and 1 control) baffled and vented culture flasks at a density of 2×10⁶ cells/mL. In experiment 1, harvesting and recirculation of spent media was performed every four days as opposed to harvesting every three days in experiment 2. 250 mL of the original culture was harvested and spun down using a Thermo Scientific Sorvall ST 16 centrifuge at 5000 rpm for 5 min for quantities over 5 mL. To the remaining 250 mL in the original culture flask, 125 mL of centrifuged supernatant was added back and complemented with 125 mL fresh media, which makes 500 mL from the combined sources. Fresh medium and concentrated cells from the preliminary culture were used strictly for the control per cycle start. Flasks were harvested and split for the next cycle at a fixed time (e.g. 9:00 am). This was repeated for five harvest cycles. Flasks were kept in a Thermo Scientific Max Q 6000 (Model, 4359) at 125 rpm shaking speed at 29.0° C.

Data was collected from each flask via 2 mL samples taken daily at a fixed time (e.g. 2:00 pm). Wet weights were obtained along with volumes of supernatant and pellet after centrifugation of the 2 mL sample using a Thermo Scientific Heraceus Pico centrifuged at 2000×g for 5 min for samples under 5 mL. 1 mL of centrifuged sample was weighed and left to dry overnight in a Lindberg/Blue (Model, G01330A-1) Oven at 60° C. Percent Moisture and final dried product weight was calculated as well. At the end of each cycle, 100 mL of culture was centrifuged and the pellet was collected along with 50 mL of supernatant for further consumption analysis.

### Experiment 3 Culture Conditions and Data Collection

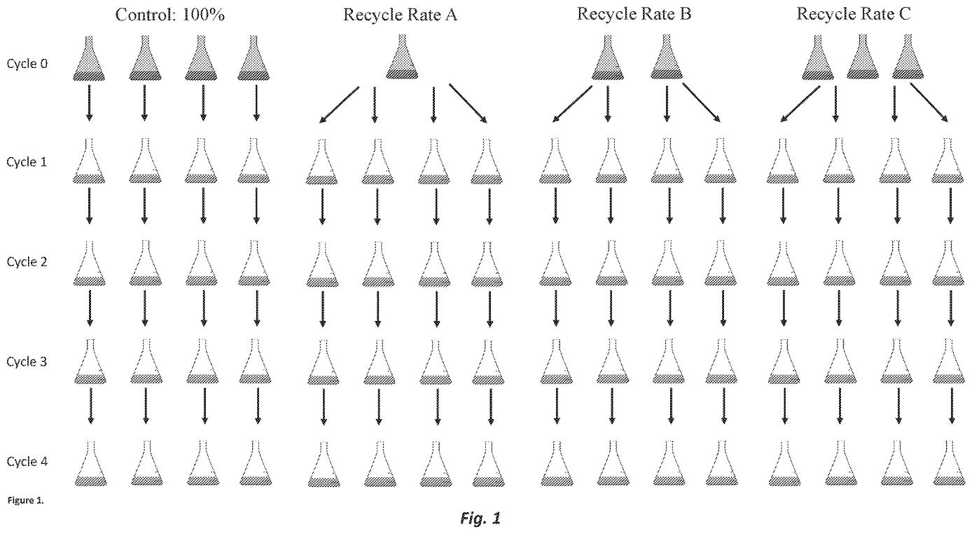

Samples in the experimental design: Control 100%, Recycle Rate A, Recycle Rate B, Recycle Rate C and cycle design were diagrammed in FIG. 1 that took place over five Cycles: 0, 1, 2, 3, and 4. A cell concentrate from the preliminary (Mother) culture was used to inoculate 8×500 mL fresh media in baffled and vented culture flasks at a density of 2×10⁸ cells/mL and left for three days as a pre-growth cycle (Cycle 0). All flasks were kept in an Adolf Kuhner AG (Model, ISF-4-V) incubator set at 100 min-1 shaking speed at 29.0° C. The experimental groups are described as follows.

Control 100%: After three days, 4 flasks were taken down by centrifugation at 5000 rpm for 5 min at room temperature. The cell pellet and supernatant was discarded. New growth media was added to the 4 flasks and 2×10⁸ cells/mL of cell inoculum was added. These cells were grown for 3 days for Cycle 1, then the process was repeated for cycles 2-4.

Recycle Rate A: After three days of growth, 1 flask in cycle 0 was taken down by centrifugation at 5000 rpm for 5 min at room temperature. The supernatant was then used to generate the hybrid culture media-25% (with 75% fresh growth media and 25% spent media) which was used as hybrid culture media for 4 flasks in Cycle 1. Culture was grown for 3 days then taken down and used as the spent media for Cycle 2. Cycles 2-4 were set up in the same way as Cycle 1.

Recycle Rate B: After three days of growth, 2 flasks in cycle 0 were taken down by centrifugation at 5000 rpm for 5 min at room temperature. The supernatant was then used to generate the hybrid culture media-50% (with 50% fresh growth media and 50% spent media) which was used as hybrid culture media for 4 flasks in Cycle 1. Culture was grown for 3 days then taken down and used as the spent media for Cycle 2. Cycles 2-4 were set up in the same way as Cycle 1.

Recycle Rate C: After three days of growth, 3 flasks in cycle 0 were taken down by centrifugation at 5000 rpm for 5 min at room temperature. The supernatant was then used to generate the hybrid culture media-75% (with 25% fresh growth media and 75% spent media) which was used as hybrid culture media for 4 flasks in Cycle 1. Culture was grown for 3 days then taken down and used as the spent media for Cycle 2. Cycles 2-4 were set up in the same way as Cycle 1.

Dried Biomass Weight

Data was collected from each flask via 25 mL samples taken daily at a fixed time (i.e. 2:00 pm), except Day Post Inoculation (DPI) 1 where 5 mL was taken. 5 mL was used for the determination of cell pellet weights. Cell pellets were formed by centrifugation at 5000 rpm for 5 min at room temperature. The supernatant was collected for further analysis. The pellet was washed twice with deionized water. Cell pellets were recovered by centrifugation at 5000 rpm for 5 min at room temperature. Samples were weighted for wet cell weight then they were freeze-dried to determine the dry cell weight. Freeze-drying the sample consisted of freezing the cell pellets at −80° C. for 10 min to up to 12 hours, then placed in a freeze-dryer under vacuum. This removed the frozen water molecules and dried biomass was left. The dried cells/biomass was then weighed to determine how much the sample weighed. The more cells or the more cellular components i.e. proteins, lipids, fatty acids and/or carbohydrates within the cell, the larger the weight.

Dried Supernatant Weight

Supernatant samples were used to measure the amount of solutes left in solution. A sample of 5 mL was collected from the culture and centrifuged at 5000 rpm for 5 min at room temperature to pellet the cells. The supernatant was collected and then filtered through a Fisherbrand Glass Fiber filter circle, G6 with a 4.25 cm diameter which is able to retain particles smaller than 3 μm. The filtered supernatant was then freeze-dried and measured in the same way as for dried biomass.

Extraction and Purification of ABA and CK's

Freeze dried cell pellets and supernatant were re-suspended in extraction buffer (CH3OH:H2O:HCOOH [15:4:1, v/v/v]) and samples were spiked with internal standards (144 ng of ²H₄-ABA (PBI, Saskatchewan, Canada) and 10 ng of each of the deuterated internal standard CKs (OlChemim Ltd, Olomouc, Czech Republic; Table 1) and then homogenized (ball mill, Retsch MM300) at 4° C. using stainless steel cylinders (RetschMM300; 5 min, 25 Hz) with zirconium oxide grinding beads (Comeau Technique Ltd., Vaudreuil-Dorion, Canada), vortexed thoroughly and sonicated for 1 min. Samples were allowed to extract passively overnight (approximately 12 hours) at −20° C. Samples were centrifuged at 1000 rpm for 10 min and supernatant collected. Pellets were re-extracted with 1 mL extraction buffer at −20° C. for 30 min. The pooled supernatants were dried in a speed vacuum concentrator at 35° C.

TABLE 1
Phytohormones (Cytokinins (CKs), and Abscisic Acid (ABA)) scanned
for by high performance liquid chromatography - tandem mass
spectrometry (HPLC-MS/MS) and associated labeled Standard

| Phytohormones | Labeled Standard |
|---|---|
| Nucleotides (NT) | |
| 1. Trans-zeatin riboside-5′-monophosphate (tZNT) | |
| 2. Cis-zeatin riboside-5′-monophosphate (cZNT) | ²H₃[9RMP]DHZ |
| 3. Dihydrozeatin riboside-5′-monophosphate (DZNT) | |
| 4. N6-isopentenyladenosine-5′monophosphate (iPNT) | ²H₆[9RMP]iP |
| Ribosides (RB) | |
| 5. Trans-zeatin riboside (tZR) | ²H₅[9R]Z |
| 6. Cis-zeatin riboside (cZR) | |
| 7. Dihydrozeatin riboside (DZR) | ²H₃[9R]DHZ |
| 8. N6-isopentenyladenosine (iPR) | ²H₆[9R]iP |
| Free bases (FB) | |
| 9. Trans-zeatin (tZ) | ²H₅Z |
| 10. Cis-zeatin (cZ) | |
| 11. Dihydrozeatin (DZ) | ²H₃DHZ |
| 12. N6-isopentenyladenine (iP) 2 H6iP | |
| Glucosides (GLUC) | |
| 13. Trans-zeatin-O-glucoside (tZOG) | ²H₅ZOG |
| 14. Cis-zeatin-O-glucoside (cZOG) | |
| 15. Dihydrozeatin-O-glucoside (DZOG) | ²H₇DHZOG |
| 16. Trans-zeatin-O-glucoside riboside (tZROG) | ²H₅ZROG |
| 17. Cis-zeatin-O-glucoside riboside cZROG | |
| 18. Dihydrozeatin-O-glucoside riboside (DZROG) | ²H₇DHZROG |
| 19. Trans-zeatin-9-glucoside (tZ9G) | ²H₅Z9G |
| 20. Cis-zeatin-9-glucoside (cZ9G) | |
| 21. Dihydrozeatin-9-glucoside (DZ9G) | ²H₃DHZ9G |
| Methylthiols (MET) | |
| 22. 2-Methylthio-trans-zeatin (2MeSZ) | ²H₅MeSZ |
| 23. 2-Methylthio-trans-zeatin riboside (2MeSZR) | ²H₅MeSZR |
| 24. 2-Methylthio-N6-isopentenyladenine (2MeSiP) | ²H₆MeSiP |
| 25. 2-Methylthio-N6-isopentenyladenosine (2MeSiPA) | ²H₆MeSiPA |
| Aromatic cytokinins | |
| 26. Benzylaminopurine (BA) | ²H₇BA |
| 27. Benzylaminopurine riboside (BAR) | ²H₇BAR |
| Abscisic Acid (ABA) | |
| 28. Abscisic Acid (ABA) | ²H₄ABA |

Extraction residues were reconstituted in 1 mL of 1 M formic acid (pH 1.4) to ensure complete protonation of all CKs. Each extract was purified on a mixed mode, reverse-phase, cation-exchange cartridge ((Canadian Life Sciences; IRIS MCX 6 mL; 25-35p 200 mg, Peterborough, ON, Canada). Cartridges were activated using 5 mL of HPLC grade methanol and equilibrated using 5 mL of 1 M formic acid (pH 1.4). After equilibration, each sample was loaded and washed with 5 mL of 1 M formic acid (pH 1.4). ABA and CKs were eluted based on their chemical properties. ABA was eluted first using 5 mL HPLC grade methanol. The nucleotide fraction (NTs) was eluted using 5 mL 0.35 M ammonium hydroxide, free bases (FBs) and ribosides (RBs) were retained based on charge and hydrophobic properties and, thus, these were eluted last using 5 mL 0.35 M ammonium hydroxide in 60% methanol. All samples were evaporated to dryness in a speed vacuum concentrator at 35° C. and immediately stored at −20° C. NTs were dephosphorylated using 3 units of bacterial alkaline phosphatase in 1 mL 0.1 M ethanolamine-HCl (pH 10.4) for 12 hours at 37° C. The resulting RBs were brought to dryness in a speed vacuum concentrator at 35° C. Samples were re-constituted in 1.5 mL double-distilled water (DDW) for further purification on a reversed-phase C18 column (Canadian Life Sciences; C18/14%, 6 ml, 500 mg; Peterborough, ON, Canada). Columns were activated using 3 mL HPLC grade methanol and equilibrated with 6 mL double-distilled water. The samples were loaded onto the C18 cartridge and allowed to pass through the column by gravity. The sorbent was washed with 3 mL DDW and analytes were eluted using 1.25 mL HPLC grade methanol. All sample eluents were dried in a speed vacuum concentrator at 35° C. and stored at −20° C. until further processing. Prior to high-performance liquid chromatography-electrospray ionization tandam mass spectrometry (HPLC-ESI MS/MS) analysis, all dried samples were re-constituted in 1.5 mL of starting conditions (CH₃COOH:CH₃CN:ddH₂O [0.08:5.0:94.92, vol/vol/vol].

CKs and ABA Quantification and Analysis

Hormones were identified and quantified by HPLC(ESI) MS/MS. A 25 μL of the sample volume was injected into a Dionex Ultimate 3000 HPLC coupled to a QExactive Orbitrap mass spectrometer (Thermo Scientific). Compounds were resolved using a reversed-phase C18 column (Kinetex 2.6u C18 100 A, 2.1×50 mm; Phenomenex, Torrance, Calif., USA). All hormone fractions were eluted with a multistep gradient of component A: Water (H₂O) with 0.08% Acetic Acid (CH₃CO₂H) mixed with component B: Acetonitrile (CH₃CN) with 0.08% CH₃CO₂H at a flow rate of 0.3 mL per minute for ABA and 0.4 mL per minute for CKs. The initial conditions were 5% B increasing linearly to 10% B over two minutes followed by an increase to 95% B over 6.5 minutes; 95% B was held constant for one and a half minutes before returning to starting conditions for five minutes. CK samples were analyzed using a Thermo Fisher Scientific QExactive Orbitrap (Santa Clara, Calif., USA) equipped with a heated electrospray ionization (HESI) source and operated in a positive ion mode, ABA samples were run in negative ion mode. To determine scanning start and end times in parallel reaction monitoring (PRM) mode the mass range in full scan mode was m/z 150-550 and resolution was set at 35,000. Temperatures of the HESI probe and capillary were 450° C. and 250° C., respectively, and the spray voltage was 3.9 kV. Sheath, auxiliary and spare gases were operated at 30, 8 and 0 arbitrary units, respectively, and the S-lens RF level was 60. PRM parameters included an automatic gain control (AGC) of 1·10⁶ and a maximum injection time (IT) of 128 msec. The precursor isolation window width was m/z 1.2 and normalized collision energies (NCE), individually optimized for each compound, ranged from 28 to 40 eV depending on the compound. Raw data files collected during LC-MS/MS analysis were processed using Thermo Fisher Scientific Xcalibur software (v. 3.0.63; Santa Clara, Calif., USA). Peaks were extracted using the accurate mass of the two most intense fragment ions and a mass tolerance of three ppm. Peak integration employed five smoothing points and a signal-to-noise (S/N) threshold of 0.5. Quantification was achieved through isotope dilution analysis based on recovery of ²H-labelled internal standards.

Endogenous CK analysis was adjusted by removing the background levels of existing endogenous hormones in the organic media (media blank), and CK concentrations were calculated using Xcalibur software package as per cell dry pellet weight. Internal standards purchased were used for the analysis (Table 1).

Glucose Consumption

A sample of the supernatant was taken and measured by an YSI analytical instrument (YSI 2700) in order to determine the amount of glucose in the sample. More specifically, 250 μL of supernatant was added to 1.75 mL of Deionized water to dilute (8 times) the sample into the range measured by the YSI instrument. The instrument has a standard that can detect glucose from 0.05 g/L to 9 g/L. It measures the glucose in an experimental sample and compares it to standard in order to determine the amount of glucose present.

Results and Discussions

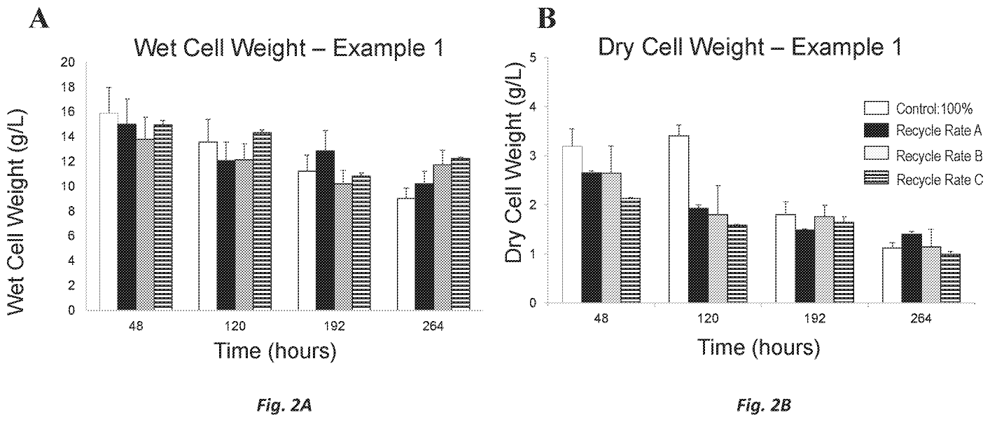

Wet and dry cell weight from different Recycle Rates and Control over time (cycles) were determined. (FIG. 2). In general, as time increased the wet cell weight decreased except for 264 hours for recycle rate B and C which increases instead. Dry weight was more variable but control in general was higher than the rest (except for 264 hours). This shows fairly consistent levels of biomass generation across recycle rates.

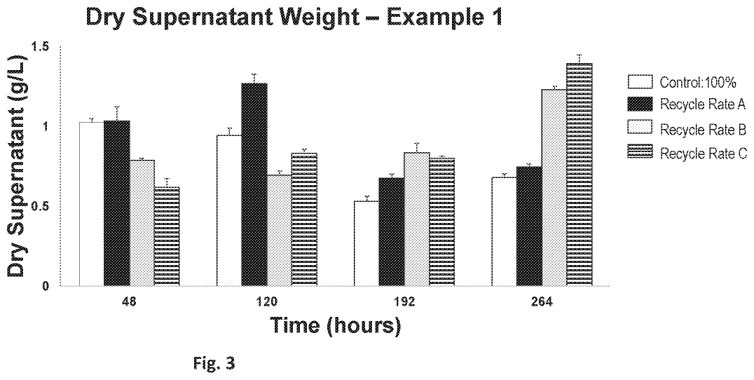

Dry supernatant weight of different experimental groups were determined. Supernatant for the Control and Recycle Rate A used less recycled culture media nutrients over time, compared to Recycled Rates B and C (FIG. 3).

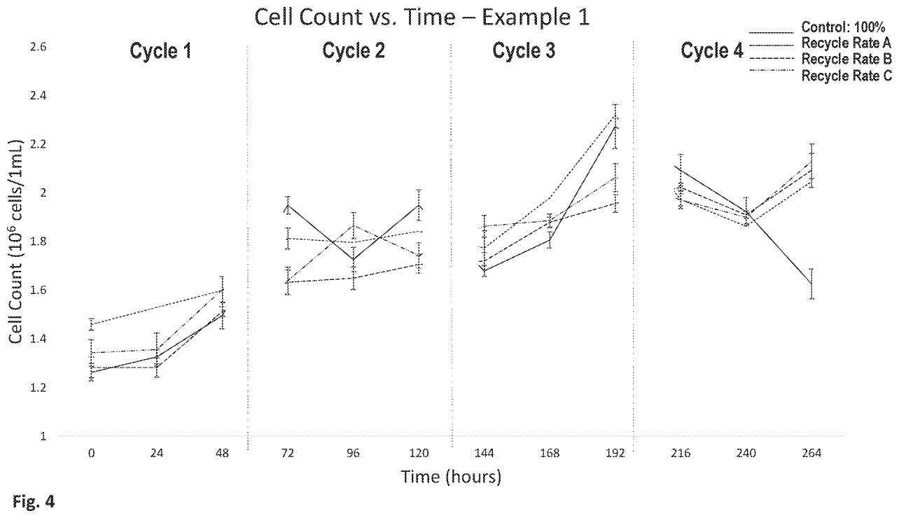

Cell counts of the different recycled culture media rates over time were determined (FIG. 4). Cell counts remain fairly similar within each cycle. This shows that cell division is not limited even at the highest recycling rate tested.

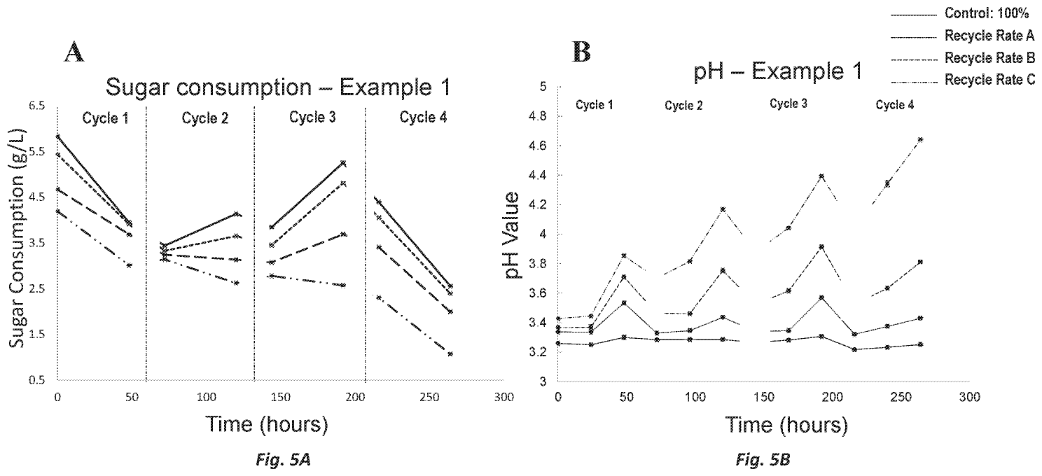

Glucose consumption from the media for each recycled rate and cycles was determined (FIG. 5A). Consumption data shows that late cycle cells use less sugar, indicating that these cells are less metabolically active. pH tracking (FIG. 5B) shows that as the recycled rate increases, the pH also increases as well as during the cycle. This shows that the control media and media in Recycle Rate A retain buffering capacity while media Recycle Rate B and C do not.

All hormone data has been corrected for the percentage of CKs and ABA present in the fresh media used in culturing. This was done by taking experimental blanks, analyzing the phytohormone levels and then subtracting these levels based on the dilution rate in the recycled media. Resulting reported hormone levels would be representative of hormones synthesized or processed by the Euglena cells themselves. However, it cannot be disregarded that the exogenous presence of hormones in the media may impact the overall endogenous profile of the Euglena cell pellet and or the hormones excreted into the media and cell free spent media used in subsequent cycling rounds.

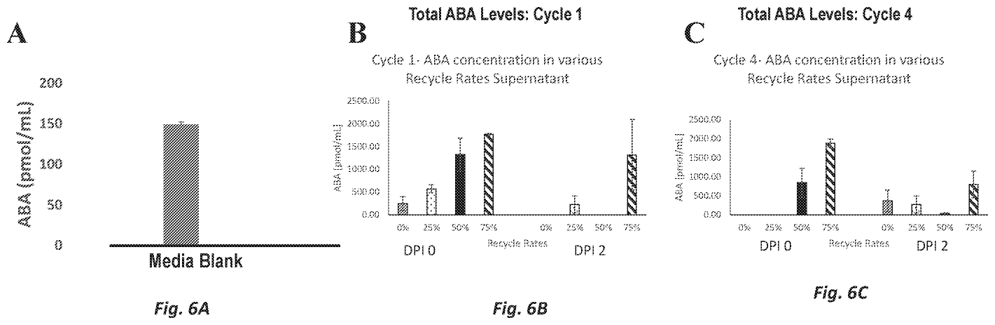

ABA analysis of the media blank and supernatant from all recycled rates was conducted (FIG. 6). The media blank accounted for −25% of the total hormone profile. The ABA data from the supernatant of cultures grown under different recycling rates showed that in Cycle 1 ABA levels in the culture media increased with higher recycling rate suggesting that Euglena may have the ability to secrete ABA into the surrounding environment. At the end of Cycle 1 ABA levels have reduced in all recycle rates by >25% of their starting values. Relative to the other recycled rates 75% had the highest level of ABA at the beginning and end of cycle 1 and the beginning and end of cycle 4, this may account for lower dry cell weights relative to other recycle rates as ABA can cause inhibition or slowing of the cell cycle and may result in increases in other metabolite production such as lipids.

Cytokinins (CK) can be broadly grouped based on their type or form. CK types include DZ, cZ, tZ and iP and CK forms include nucleotide, riboside, freebase, glucoside and methylthiol. Broadly the CK types include the various forms ie DZ type: DZNT, DZR, DZ; cZ type: cZNT, cZR, cZ; tZ type: tZNT, tZR, tZ and iP type: iPNT, iPR, iP. CK forms incorporate the various CK types. The CK analysis will examine the CK profiles of the supernatant of all recycle rates and provide analysis based on the CK type and form.

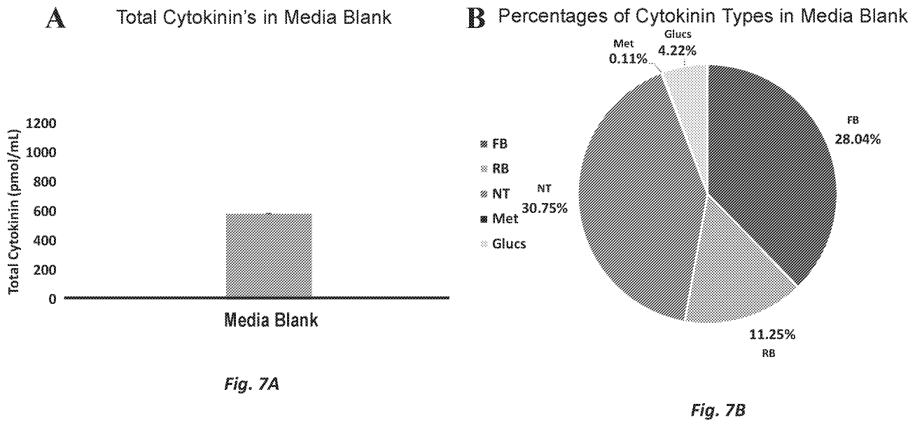

Total cytokinin levels in the media blank as well as the cytokinin types in the media blank were determined (FIG. 7). Total CK concentrations as well as the types and forms present, were determined for the media blank. The majority of the CKs present were the nucleotide form followed by free base and riboside forms. The dominant CK types included iP and DZ.

Cytokinin measurements are reported in Tables 2-3 for supernatant measurements and Tables 4-5 for the cell pellet cytokinin measurements.

TABLE 2
Cytokinin and ABA concentrations (pmol/mL) from the supernatant of E. gracilis cultures
grown at varying recycled rates of cell free spent media. Values are means (n = 3)
taken at specific timepoints during cultivation of cycle 1 at the beginning (DPI 0) and
the end (DPI 2) of the growth cycle. Empty cells indicate values of zero for those analytes.

| | Cycle | | | | | | | |
|---|---|---|---|---|---|---|---|---|
| | 1 | | | | | | | |
| | DPI | | | | | | | |
| | 0 | | | | 2 | | | |
| Recycle Rate | 0% | 25% | 50% | 75% | 0% | 25% | 50% | 75% |
| ABA | 242.41 | 570.92 | 1318.74 | 1770.41 | — | 227.51 | — | 1316.60 |
| Free base | | | | | | | | |
| DZ | | | | 91.26 | | 25.80 | 11.40 | 32.96 |
| tZ | | 43.67 | 49.67 | | 23.40 | 33.83 | 89.05 | 220.12 |
| cZ | 240.35 | 539.98 | 717.14 | 612.52 | 291.04 | 1004.14 | 1163.08 | 1853.12 |
| iP | | 447.71 | 894.16 | 1123.03 | 2.92 | 206.88 | 554.24 | 1358.09 |
| Riboside | | | | | | | | |
| DZR | 230.35 | 256.13 | 331.39 | 389.46 | 79.92 | 199.47 | 308.83 | 557.30 |
| tZR | | | | | | | | |
| cZR | | 86.81 | 147.50 | 121.81 | 74.44 | 78.86 | 147.50 | 297.89 |
| iPR | 3934.7 | 5181.6 | 5122.00 | 5934.24 | 1446.29 | 3115.14 | 3447.73 | 6962.65 |
| Nucleotide | | | | | | | | |
| DZNT | 66.87 | 276.14 | 478.32 | 603.06 | 97.80 | 239.52 | 330.09 | 671.85 |
| tZNT | 77.38 | 60.34 | 53.28 | 40.40 | 33.82 | 39.25 | 30.41 | 52.46 |
| cZNT | 89.92 | 92.51 | 92.28 | 92.39 | 68.72 | 86.42 | 94.58 | 165.28 |
| iPNT | | | 8021.6 | 4687.9 | | | 2624.5 | 2822.4 |
| Methylthiol | | | | | | | | |
| 2MeSZ | | 6.85 | 206.80 | 190.89 | 0.55 | 67.91 | 279.06 | 321.41 |
| 2MeSiP | 4.43 | 10.37 | 16.01 | 2.42 | 0.63 | | | 12.13 |
| 2MeSZR | 39.52 | 51.59 | 59.95 | 70.98 | 42.66 | 81.30 | 104.43 | 210.70 |
| 2MeSiPA | | 10.28 | 19.18 | 23.92 | 13.78 | 20.68 | 24.41 | 42.32 |
| Glucoside | | | | | | | | |
| DZOG | | | | | | | | |
| DZROG | | | | | | | | |
| tZROG | | | | | | | | |
| cZROG | 142.32 | 143.57 | 365.24 | 517.65 | 38.36 | 165.80 | 701.00 | 947.58 |
| DZ9G | 1659.03 | 2903.76 | 8167.62 | | 1607.42 | 2872.81 | 6365.61 | 3457.62 |
| tZOG | | | | | 718.80 | 16.77 | 11.76 | 271.53 |
| cZOG | | | | | 899.21 | 367.56 | 396.98 | 1095.97 |
| tZ9G | 55.58 | 41.14 | 16.91 | 44.92 | 14.08 | 11.93 | 33.93 | 16.91 |
| cZ9G | | | 8.96 | | 3.16 | 9.40 | 5.65 | 4.34 |
| Total types | | | | | | | | |
| DZ Types | 297.22 | 532.26 | 809.72 | 1083.78 | 177.72 | 464.79 | 650.32 | 1262.12 |
| tZ types | 77.38 | 104.01 | 102.94 | 40.40 | 57.22 | 73.08 | 119.45 | 272.58 |
| cZ types | 330.28 | 719.30 | 956.92 | 826.72 | 434.20 | 1169.42 | 1405.16 | 2316.28 |
| iP types | 3934.70 | 5629.34 | 14037.71 | 11745.20 | 1449.21 | 3322.03 | 6626.51 | 11143.13 |
| Total forms | | | | | | | | |
| FBRNT | 4639.57 | 6984.90 | 15907.29 | 13696.10 | 2118.35 | 5029.31 | 8801.46 | 14994.11 |
| Glucosides | 1856.94 | 3088.48 | 8558.73 | 562.57 | 3281.02 | 3444.28 | 7514.92 | 5793.95 |
| Methylthiols | 43.95 | 79.09 | 301.94 | 288.21 | 57.62 | 169.88 | 407.90 | 586.56 |
| Total CKs | 6540.46 | 10152.48 | 24767.96 | 14546.88 | 5456.99 | 8643.48 | 16724.27 | 21374.62 |

TABLE 3
Cytokinin and ABA concentrations (pmol/mL) from the supernatant of E. gracilis cultures
grown at varying recycled rates of cell free spent media. Values are means (n = 3)
taken at specific timepoints during cultivation of cycle 4 at the beginning (DPI 0) and
the end (DPI 2) of the growth cycle. Empty cells indicate values of zero for those analytes.

| | Cycle | | | | | | | |
|---|---|---|---|---|---|---|---|---|
| | 4 | | | | | | | |
| | DPI | | | | | | | |
| | 0 | | | | 2 | | | |
| Recycle Rate | 0% | 25% | 50% | 75% | 0% | 25% | 50% | 75% |
| ABA | | | 843.99 | 1876.96 | 362.06 | 271.83 | 30.53 | 799.47 |
| Free base | | | | | | | | |
| DZ | 85.15 | 59.70 | 35.96 | 25.39 | 22.87 | 28.70 | 14.22 | 45.84 |
| tZ | | | 130.25 | 128.23 | 13.07 | 46.48 | 149.77 | 80.54 |
| cZ | 1600.24 | 1959.34 | 990.68 | 938.20 | 552.97 | 257.78 | 1083.38 | 489.14 |
| iP | 49.79 | 919.16 | 614.37 | 1229.42 | 92.43 | | 857.43 | 1029.39 |
| Riboside | | | | | | | | |
| DZR | 207.55 | 352.03 | 332.73 | 579.24 | 129.85 | 126.70 | 505.49 | 365.09 |
| tZR | | | | | | | | |
| cZR | | 29.15 | 173.59 | 304.82 | 19.00 | 37.62 | 304.74 | 331.84 |
| iPR | 3699.7 | 3883.14 | 3820.37 | 7056.04 | 1565.21 | 545.82 | 5069.34 | 4368.42 |
| Nucleotide | | | | | | | | |
| DZNT | 68.67 | 442.34 | 305.07 | 766.81 | 55.74 | 51.94 | 574.51 | 683.13 |
| tZNT | 73.60 | 53.03 | 26.85 | 42.72 | 37.26 | 21.18 | 40.07 | 39.68 |
| cZNT | 76.71 | 80.59 | 58.10 | 191.03 | 66.88 | 53.27 | 143.46 | 227.62 |
| iPNT | | | | 7712.5 | | | 873.3 | 5055.9 |
| Methylthiol | | | | | | | | |
| 2MeSZ | 888.24 | 443.71 | 373.09 | 245.66 | 96.35 | 452.78 | 438.07 | 377.80 |
| 2MeSiP | 187.76 | 173.05 | 52.87 | 19.95 | 26.24 | 28.49 | 63.39 | 37.31 |
| 2MeSZR | 42.01 | 103.70 | 265.11 | 253.36 | 70.06 | 106.36 | 236.59 | 166.82 |
| 2MeSiPA | | 9.62 | 19.71 | 22.91 | 11.12 | 11.22 | 24.52 | 22.93 |
| Glucoside | | | | | | | | |
| DZOG | | | | 28.74 | | | | |
| DZROG | | | | | | | | |
| tZROG | | | | | | | | |
| cZROG | 661.22 | 446.07 | 542.57 | 1020.62 | 282.78 | 583.74 | 800.51 | 693.27 |
| DZ9G | 1622.40 | 1580.05 | 3879.76 | 7969.48 | 946.16 | 925.76 | 3029.74 | 782.32 |
| tZOG | 132.93 | 237.71 | 223.74 | 60.47 | 72.30 | | 172.98 | 141.68 |
| cZOG | 590.59 | 1267.82 | 1121.69 | 664.46 | 137.30 | 324.10 | 834.10 | 488.62 |
| tZ9G | 21.37 | 23.61 | 10.21 | 17.09 | | 13.35 | 37.29 | 29.34 |
| cZ9G | 1.73 | 1.41 | 5.82 | 9.38 | | 0.54 | 3.45 | 1.49 |
| Total types | | | | | | | | |
| DZ Types | 361.37 | 854.08 | 673.77 | 1371.44 | 208.45 | 207.34 | 1094.22 | 1094.06 |
| tZ types | 73.60 | 53.03 | 157.10 | 170.95 | 50.33 | 67.66 | 189.84 | 120.22 |
| cZ types | 1676.95 | 2069.09 | 1222.37 | 1434.04 | 638.85 | 348.67 | 1531.59 | 1048.59 |
| iP types | 3749.52 | 4802.29 | 4434.73 | 15998.00 | 1657.64 | 545.82 | 6800.08 | 10453.70 |
| Total forms | | | | | | | | |
| FBRNT | 5861.43 | 7778.48 | 6487.97 | 18974.43 | 2555.27 | 1169.49 | 9615.73 | 12716.58 |
| Glucosides | 3030.24 | 3556.66 | 5783.78 | 9770.24 | 1438.54 | 1847.49 | 4878.07 | 2136.72 |
| Methylthiols | 1118.02 | 730.07 | 710.78 | 541.89 | 203.77 | 598.86 | 762.57 | 604.86 |
| Total CKs | 10009.69 | 12065.22 | 12982.53 | 29286.56 | 4197.58 | 3615.84 | 15256.36 | 15458.15 |

TABLE 4
Cytokinin and ABA concentrations (pmol/gDCW) from the pellet of E. gracilis
cultures grown at varying recycled rates of cell free spent media. Values
are means (n = 3) taken at specific timepoints during cultivation
of cycle 1 at the beginning (DPI 0) and the end (DPI 2) of the growth
cycle. Empty cells indicate values of zero for those analytes.

| | Cycle | | | | | | | |
|---|---|---|---|---|---|---|---|---|
| | 1 | | | | | | | |
| | DPI | | | | | | | |
| | 0 | | | | 2 | | | |
| Recycle Rate | 0% | 25% | 50% | 75% | 0% | 25% | 50% | 75% |
| ABA | | | | | | | | |
| Free base | | | | | | | | |
| DZ | 0.40 | 0.21 | 0.21 | 0.33 | 0.51 | 0.31 | 0.72 | 0.62 |
| tZ | | | | | | | | |
| cZ | 98.18 | 85.75 | 96.11 | 198.22 | 219.39 | 134.10 | 57.64 | 47.05 |
| iP | 56.68 | 88.07 | 49.40 | 238.33 | 37.40 | 75.88 | 75.06 | 71.82 |
| Riboside | | | | | | | | |
| DZR | 5.88 | 3.80 | 3.80 | 3.48 | 3.15 | 4.27 | 5.38 | 6.65 |
| tZR | 2.20 | 1.44 | 1.15 | 1.25 | 2.19 | 2.49 | 1.73 | 1.89 |
| cZR | 1.31 | 1.14 | 1.87 | 2.17 | 1.21 | 1.99 | 3.50 | 6.74 |
| iPR | 112.2 | 64.9 | 64.48 | 55.25 | 44.86 | 42.75 | 97.37 | 125.44 |
| Nucleotide | | | | | | | | |
| DZNT | 12.41 | 7.65 | 8.43 | 7.50 | 9.03 | 9.09 | 15.20 | 17.11 |
| tZNT | 18.35 | 7.64 | 7.39 | 3.96 | 5.03 | 4.13 | 6.77 | 16.39 |
| cZNT | 14.05 | 11.93 | 12.91 | 12.97 | 27.51 | 26.49 | 35.31 | 58.74 |
| iPNT | 373.46 | 478.9 | 455.1 | 494.3 | 703.2 | 582.4 | 795.0 | 722.6 |
| Methylthiol | | | | | | | | |
| 2MeSZ | 20.96 | 18.88 | 19.28 | 16.24 | 10.72 | 12.33 | 15.18 | 21.01 |
| 2MeSiP | 48.41 | 49.86 | 39.42 | 29.67 | 24.71 | 26.46 | 38.78 | 48.46 |
| 2MeSZR | 3.91 | 4.45 | 3.13 | 3.50 | 2.88 | 3.36 | 7.44 | 12.16 |
| 2MeSiPA | 1.12 | 0.41 | 1.42 | 0.20 | 0.24 | 0.31 | 0.64 | 0.51 |
| Glucoside | | | | | | | | |
| DZOG | | 0.04 | 0.12 | | 0.49 | | | |
| DZROG | 0.07 | | 0.04 | 0.04 | 0.09 | 0.02 | 0.04 | 0.05 |
| tZROG | 0.24 | 0.29 | 0.07 | 0.07 | 1.17 | 0.87 | 1.09 | 1.08 |
| cZROG | 7.20 | 4.90 | 5.61 | 4.94 | 4.30 | 4.00 | 7.37 | 8.61 |
| DZ9G | | | | | | | | |
| tZOG | 1.86 | 1.57 | 0.54 | 1.99 | 0.83 | 2.00 | 0.06 | 1.71 |
| cZOG | 3.10 | 1.00 | 1.73 | 1.60 | 1.38 | 1.73 | 3.36 | 3.19 |
| tZ9G | | | | | | | | |
| cZ9G | | | | | | | | |
| Total types | | | | | | | | |
| DZ Types | 18.69 | 11.66 | 12.44 | 11.31 | 12.69 | 13.67 | 21.30 | 24.38 |
| tZ types | 20.55 | 9.08 | 8.54 | 5.21 | 7.22 | 6.62 | 8.50 | 18.28 |
| cZ types | 113.54 | 98.82 | 110.89 | 213.36 | 248.11 | 162.58 | 96.45 | 112.53 |
| iP types | 542.38 | 631.85 | 568.94 | 787.85 | 785.43 | 700.99 | 967.39 | 919.90 |
| Total forms | | | | | | | | |
| FBRNT | 695.16 | 751.41 | 700.81 | 1017.73 | 1053.45 | 883.86 | 1093.64 | 1075.09 |
| Glucosides | 12.47 | 7.80 | 8.11 | 8.64 | 8.26 | 8.62 | 11.92 | 14.64 |
| Methylthiols | 74.40 | 73.60 | 63.25 | 49.61 | 38.55 | 42.46 | 62.04 | 82.14 |
| Total CKs | 782.03 | 832.81 | 772.17 | 1075.98 | 1100.26 | 934.94 | 1167.60 | 1171.87 |

TABLE 5
Cytokinin and ABA concentrations (pmol/gDCW) from the pellet of E. gracilis
cultures grown at varying recycled rates of cell free spent media. Values
are means (n = 3) taken at specific timepoints during cultivation
of cycle 4 at the beginning (DPI 0) and the end (DPI 2) of the growth
cycle. Empty cells indicate values of zero for those analytes.

| | Cycle | | | | | | | |
|---|---|---|---|---|---|---|---|---|
| | 4 | | | | | | | |
| | DPI | | | | | | | |
| | 0 | | | | 2 | | | |
| Recycle Rate | 0% | 25% | 50% | 75% | 0% | 25% | 50% | 75% |
| ABA | | | | | | | | |
| Free base | | | | | | | | |
| DZ | 0.61 | 0.96 | 1.10 | 0.70 | 0.33 | 0.79 | 1.48 | 0.87 |
| tZ | | | | | | | | |
| cZ | 27.62 | 54.26 | 31.40 | 24.43 | 77.57 | 61.32 | 20.49 | 13.99 |
| iP | 65.36 | 83.66 | 89.59 | 64.76 | 34.68 | 54.58 | 108.74 | 85.37 |
| Riboside | | | | | | | | |
| DZR | 4.00 | 6.04 | 6.43 | 5.08 | 2.50 | 5.48 | 6.38 | 4.20 |
| tZR | 1.39 | 2.68 | 1.49 | 0.87 | 1.97 | 2.49 | 1.75 | 0.47 |
| cZR | 1.11 | 3.49 | 3.55 | 3.77 | 1.19 | 2.78 | 4.51 | 3.95 |
| iPR | 87.3 | 89.06 | 106.48 | 94.52 | 39.47 | 72.76 | 101.97 | 91.43 |
| Nucleotide | | | | | | | | |
| DZNT | 10.28 | 15.24 | 13.81 | 13.68 | 7.59 | 13.36 | 20.36 | 16.04 |
| tZNT | 13.42 | 16.91 | 13.52 | 10.07 | 6.61 | 9.08 | 8.92 | 9.05 |
| cZNT | 20.63 | 23.37 | 22.72 | 32.74 | 23.81 | 27.73 | 35.68 | 51.89 |
| iPNT | 602.0 | 490.0 | 459.1 | 719.0 | 528.6 | 608.5 | 997.7 | 992.3 |
| Methylthiol | | | | | | | | |
| 2MeSZ | 16.27 | 19.95 | 13.98 | 15.97 | 9.24 | 15.48 | 21.44 | 24.55 |
| 2MeSiP | 46.76 | 46.35 | 34.54 | 44.07 | 24.51 | 122.72 | 234.44 | 208.35 |
| 2MeSZR | 5.21 | 5.71 | 6.88 | 11.65 | 3.62 | 8.33 | 42.21 | 44.82 |
| 2MeSiPA | 0.15 | | 0.66 | 0.75 | 0.62 | 0.83 | 3.77 | |
| Glucoside | | | | | | | | |
| DZOG | | | | | | | | |
| DZROG | | 0.04 | 0.03 | 0.04 | 0.03 | 0.04 | | |
| tZROG | | 0.09 | 0.12 | 0.21 | 0.45 | 0.17 | 0.74 | 0.44 |
| cZROG | 6.63 | 6.49 | 7.48 | 7.68 | 4.15 | 6.01 | 8.76 | 7.75 |
| DZ9G | | | | | | | | |
| tZOG | 1.33 | 0.82 | 0.61 | 1.63 | 1.12 | 0.85 | 2.57 | 1.58 |
| cZOG | 2.04 | 2.29 | 4.05 | 3.10 | 1.93 | 3.11 | 4.36 | 4.05 |
| tZ9G | | | | | | | | |
| cZ9G | | | | | | | | |
| Total types | | | | | | | | |
| DZ Types | 14.89 | 22.24 | 21.34 | 19.46 | 10.42 | 19.63 | 28.22 | 21.11 |
| tZ types | 14.81 | 19.59 | 15.01 | 10.94 | 8.58 | 11.57 | 10.67 | 9.52 |
| cZ types | 49.36 | 81.12 | 57.67 | 60.94 | 102.57 | 91.83 | 60.68 | 69.83 |
| iP types | 754.68 | 662.69 | 655.19 | 878.26 | 602.70 | 735.86 | 1208.40 | 1169.13 |
| Total forms | | | | | | | | |
| FBRNT | 833.74 | 785.64 | 749.21 | 969.60 | 724.27 | 858.89 | 1307.97 | 1269.59 |
| Glucosides | 10.00 | 9.73 | 12.29 | 12.66 | 7.68 | 10.18 | 16.43 | 13.82 |
| Methylthiols | 68.39 | 72.01 | 56.06 | 72.44 | 37.99 | 147.36 | 301.86 | 277.72 |
| Total CKs | 912.13 | 867.38 | 817.56 | 1054.70 | 769.94 | 1016.43 | 1626.26 | 1561.13 |

Total cytokinin levels were tested in both the media supernatant, as well was in the cell pellets. In general, higher ribosides are observed at the beginning of the cycle, with the glucoside form (storage form) found at the end of the cycles. In terms of the glucosides, there is an increase in the cZ type. As well, it is mainly cZ and iP forms.

As there is an increase in glucoside forms as the end of the cycle, at the beginning of the next cycle, the higher the recycled rate, the more glucosides present at the start of the cycle. The most common type of cytokinin was the DZ form glucoside with Recycle Rate B (50%) and Rate C (75%) having the higher levels of the cytokinin. However, the levels reduce over the timespan of the cycle.

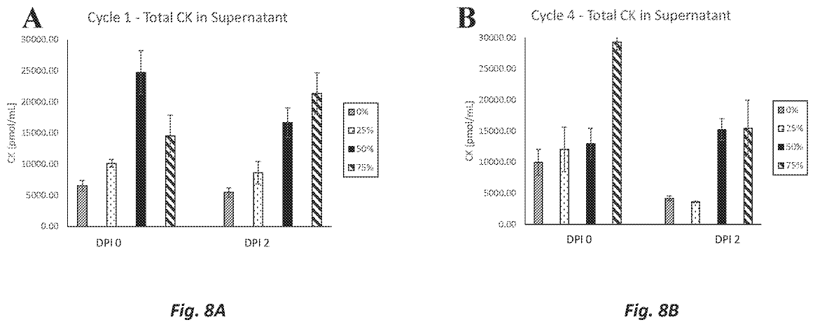

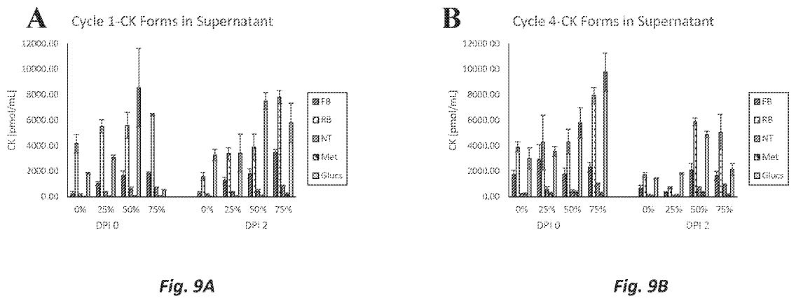

Cytokinin levels in the 100% control supernatant were determined (FIG. 8-9). Total cytokinin levels decreased from Cycle 1 to Cycle 4. CK levels in the 100% fresh media control was determined during E. gracilis culture growth and background CK levels from the media blank were removed from further calculations. Total CKs decreased between the beginning and end of cycle 1 and cycle 4. Decreases in cycle 1 showed reduction in the dominant iP type CK, specifically iPR. Increases in cZ type were mainly due to increases in cZR and cZ. CK forms at the beginning of cycle 1 were dominated by ribosides whereas at the end of cycle 1 ribosides had decreased and an increased proportion of glucosides were present likely due to a reduction in DZR and iPR and increase in tZOG and cZOG. As tZOG and cZOG act as storage forms, they can be found to increase with increases in their unconjugated forms ie. cZ and tZ which were both found to increase between the beginning and end of cycle 1. Cycle 4 followed a similar trend with decreases in total CKs and similar reduction in iP CK types.

Total CK levels in the media from Recycle Rate A (25% hybrid culture media) supernatant were determined (FIG. 8-9). The total CK level decreased from the beginning of Cycle 1 to the end of cycle 1, this trend was also reflected in cycle 4. With E. gracilis growth there is a change in CK profiles in which overall CK ribosides are reduced from the beginning to end of cycle 1 this is due to the reduction in iP CK types and the reduction in iP and iPR. CK glucosides and CKRB forms become more equivalent in the profile. Other CK types, including DZ and tZ, types are also reduced from the beginning to the end of cycle 1 this trend is also reflected in cycle 4. However, cZ types increase between the beginning and end of Cycle 1 due to the increased abundance of cZ. Increase in cZ glucosides likely to counterbalance the increase in cZ forms from the beginning to the end of cycle 1.

Total CK levels in the supernatant from Recycle Rate B (50% hybrid culture media) were determined (FIG. 8-9). The total CK levels decreased from the beginning to the end of cycle 1, this trend was not noted in cycle 4. Increase in cZ types with the increase in cZ additionally there is an increase in cZ type glucosides (cZOG and cZROG).

There is a reduction in iP CK types as seen in other recycle rates however there are higher levels of glucosides in the form of DZ types, while reduced from the beginning to the end of cycle 1 they are a dominant type in the CK profile.

These differences in the overall CK form dominance may be correlated with growth trends including DCW that may be related to the change in CK profile noted in this treatment.

Total CK levels in the supernatant from Recycle Rate C (75% hybrid culture media) were determined (FIG. 8-9). The total CK levels increased in cycle 1 from the beginning to the end, less change was noted for cycle 4. Does not have the characteristics reduction in iP types as seen in other recycle rates. There is an increase in other CK types relative to the iP types. Increase in CK glucoside forms as well as riboside forms in cycle 1, these increases are not reflected in cycle 4.

Overall trends show different CK profiles for the different media from Recycle Rate A, B and C. With the background base media hormone profile correction incorporated it can be noted that the media blank levels were lower than the measured levels in the recycled culture media and control samples. Furthermore, analysis of the changes in CK profiles highlights conserved trends in various culture conditions. Overall there is trend towards decreased riboside forms including iP types as well as an increase in cZ types with increased culturing time. This indicates that E. gracilis is capable of synthesizing and excreting certain CKs into the media. Additionally, CK glucosides increase in dominance at the end of cycles indicating the ability of E. gracilis to process CKs into stable glucoside forms.

Profiles provide data that will help to guide production. Matching hormone data and modifying E. gracilis output to match the most productive profile will guide culturing conditions and techniques. Ideally parameters will reduce glucoside abundance and also highlight the importance of iP and cZ type CKs in growth processes.

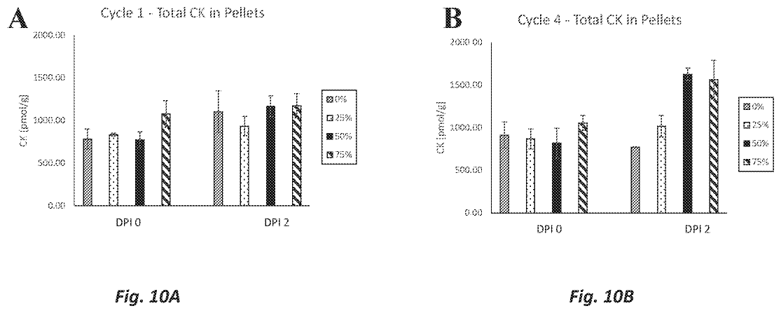

In terms of the total CK's for the pellet, the endogenous cytokinins increases by the end of the cycle, with the higher recycling rates (50% and 75%) showing the higher levels of cytokinins (FIG. 10). No ABA was detected in any pellet sample.

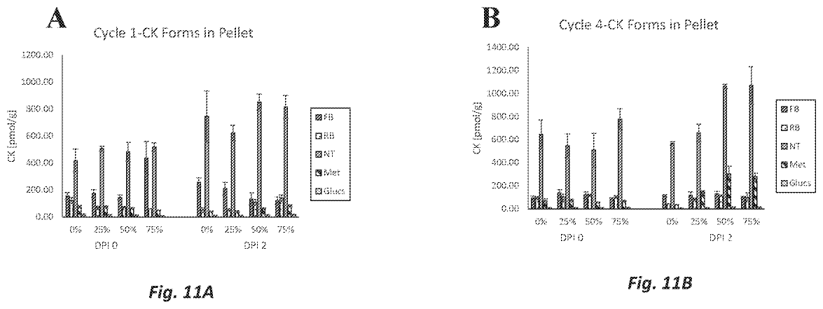

Total CK levels in the pellet from 100% fresh media were determined (FIG. 10-11). The total CK levels increased overall from the beginning to the end of cycle 1. Decreases in DZ types were found between the beginning and end of cycle 1 and 4 due to a reduction in DZR and DZNT forms. tZ types decreased in cycle 1 and 4 with a reduction in tZNT. cZ types increased with increases in cZ and cZNT, this increase in cZ types is also seen in cycle 4. iP types increased due to the abundance of iPNT but there was an overall reduction in iPR and iP. Of the methylthiol CKs 2MeSiP was reduced by 50% during growth in both cycle 1 and cycle 4. CK glucoside forms are reduced between the beginning and end of cycle 1 and 4, interestingly this endogenous reduction can be paired with in exogenous increase in CK glucosides in the corresponding supernatant sample.

Total CK levels in the pellet from Recycle Rate A (25% hybrid culture media) were determined (FIG. 10-11). CK levels increased from the beginning to the end of both cycle 1 and cycle 4. This increase was due to increases in cZ and iP CK types. Specifically, cZ, cZNT and iPNT accumulation over the growth cycle. Of the methythiol CKs 2MeSiP was reduced between the beginning and end of cycle 1 whereas it increased between the beginning and end of cycle 4. This increased accumulation during cycle 4 correlates with a decrease in overall biomass for Recycle Rate A during cycle 4. Recycle Rate B and C also show a similar increase in 2MeSiP in cycle 4 and this may be correlated to DCW. Glucosides were also detected in the pellets of recycle rate A there was no distinct changes during growth.

Total CK levels in the pellet from Recycle Rate B (50% hybrid culture media) were determined (FIG. 10-11). Total CK levels increased in the pellet for recycle rate B this was due to the increase in CK iP CK types. The main iP CK type accumulation was iPNT followed by iPR and iP. cZ types decreased which is different from what was seen in the 100% fresh media and recycle rate A. cZ CK type decrease was due to a decrease in cZ whereas there was an increase in cZNT (precursor form).

Total CK levels in the pellet from Recycle Rate C (75% hybrid culture media) were determined (FIG. 10-11). Total CKs increased between the beginning and end of cycle 1 and cycle 4. Increases were seen in the CK forms particularly in the nucleotide form as well as the methylthiol CKs. Increases could be seen in cZNT and iPNT, and 2MeSiP and 2MeSZR from the beginning to end of cycle 1 and 4. There were also decreases in decreases in cZ and iP in cycle 1 which was seen in Recycle rate B as well.

Overall there was an increase in endogenous CKs between the beginning and end of cycles throughout the recycle rates. 100% fresh and recycle rate A produced increased levels of cZ whereas B and C had reduced levels of cZ overall in cycle 1 which may correlate to the growth difference seen in these cells.

Overall there was an increased accumulation of iP types in pellets in the form of iPNT, these iP types may be produced in the cell (NT) or be taken up and processed by the cell from the surrounding media.

Additionally, methylthiol CKs may play a role in overall growth rate of the culture; 2MeSiP decreases in 100% fresh media for both cycle 1 and 4, in recycle rate A the methylthiol 2MeSiP was reduced between the beginning and end of cycle 1 whereas it increased between the beginning and end of cycle 4. This increased accumulation during cycle 4 correlates with a decrease in overall biomass for Recycle Rate A during cycle 4. Recycle Rate B and C also show a similar increase in 2MeSiP in cycle 4 and this may be correlated to DCW.

In the higher recycling rate there is a reduction on the cZ types of cytokinins. This may correlate with the cZ forms being secreted into the media as noted in the supernatant observations. In all rates, there is an increase in the iP types in the pellet, in particular the nucleotide form. Without wishing to be bound by theory, this maybe by that the iP types are produced in the cell in NT form, or are taken up and processed by the cell.

### Example 2: Hybrid Culture Media B

Introduction

Further to Example 1 that used molasses as a carbohydrate source, glucose as a specific sugar source was also tested.

Methods and Materials

Euglena gracilis Strain Z

Euglena gracilis strain Z was used as the cell culture. Starting cell culture: Cells were inoculated into a growth media containing: Glucose, potassium phosphate, magnesium phosphate, calcium chloride, various trace metals, ammonium sulfate as well as the vitamins Biotin (B7), Thiamine HCl (B1), B6 and B12 (see Table 6) at a concentration of 2×10⁶ cells/mL.

TABLE 6
Components of the fresh growth media used to
grow Euglena gracilis, pH adjusted to 3.2.

| | Final concentration |
|---|---|
| Fresh Growth Media | in media (g/L) |
| KH₂PO₄ | 5 |
| MgSO₄•7H₂O | 5 |
| CaCl₂•2H2O | 0.5 |
| Glucose | 20 |
| (NH₄)₂SO₄ | 2 |
| Iron(III) Chloride (FeCl₃•6 H₂O) | 0.0196 |
| Manganese Chloride (MnCl₂•4 H₂O) | 0.0036 |
| Zinc sulphate (ZnSO₄•7 H₂O) | 0.0022 |
| Cobalt Chloride (CoCl₂•6 H₂O) | 0.0004 |
| Sodium Molybdate (Na₂MoO₄•2 H₂O) | 0.00025 |
| Sodium EDTA (Na₂EDTA•2 H₂O) | 0.1 |
| Biotin | 0.0001 |
| Thiamine HCl | 0.01 |
| Vitamin B6 | 0.002 |
| Vitamin B12 | 0.00005 |

Generation of Recycled Culture Media

The cells were grown for 3-4 days until the glucose was exhausted (reached approximately 0 g/L in the media). This generated the first spent media that was used to make the recycled culture media for the first recycled growth cycle (Cycle 1).

Conditions Tested

Four different conditions were tested in this experiment:

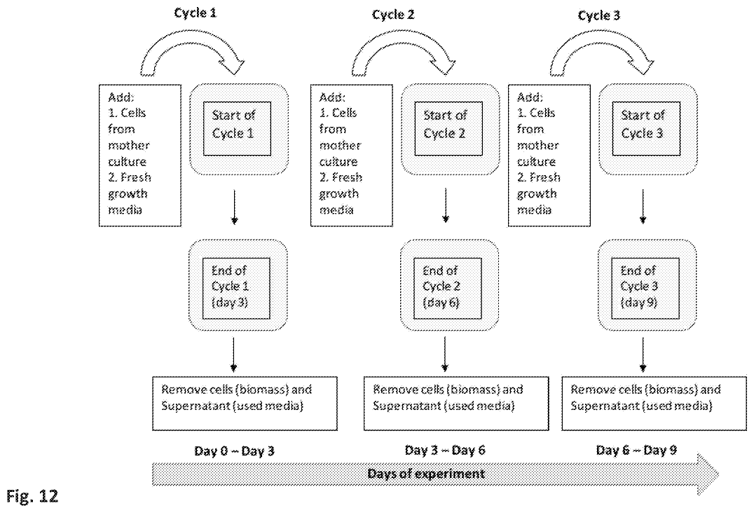

Condition 1: Cells from a mother culture (starting concentration of 2 million cells/mL) were incubated in fresh growth media and grown for 3 days. The flasks were restarted with fresh growth media and fresh cell inoculum and Cycle 2 started. This Cycle 2 ended again after 3 days. A Cycle 3 was repeated in the same way (FIG. 12).

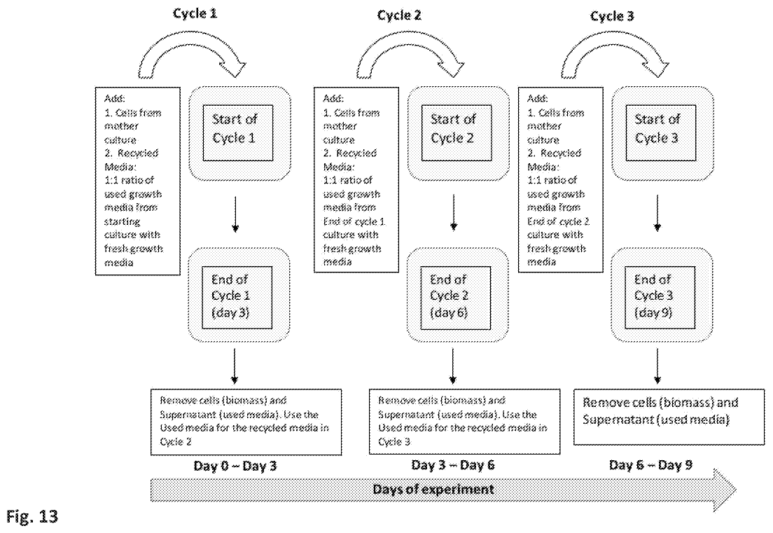

Condition 2: Cells from a mother culture (starting concentration of 2 million cells/mL) were incubated in a recycled growth media and grown for 3 days for Cycle 1 (see FIG. 13). The recycled growth media was made up of spent growth media and fresh growth media in a 1:1 ratio. For Cycle 2, the spent growth media from Cycle 1 was mixed with fresh growth media at 1:1 ratio to generate a recycled culture media for Cycle 2. Fresh cells were inoculated (2 million cells/mL) into the recycled culture media and the cells were grown for 3 days. For Cycle 3, the spent growth media from Cycle 2 was mixed with fresh growth media at 1:1 ratio to generate a recycled culture media for Cycle 3. Fresh cells were inoculated (2 million cells/mL) into the recycled culture media and the cells were grown for 3 days. The skilled person can readily recognize that additional cycles can be carried out in a similar manner.

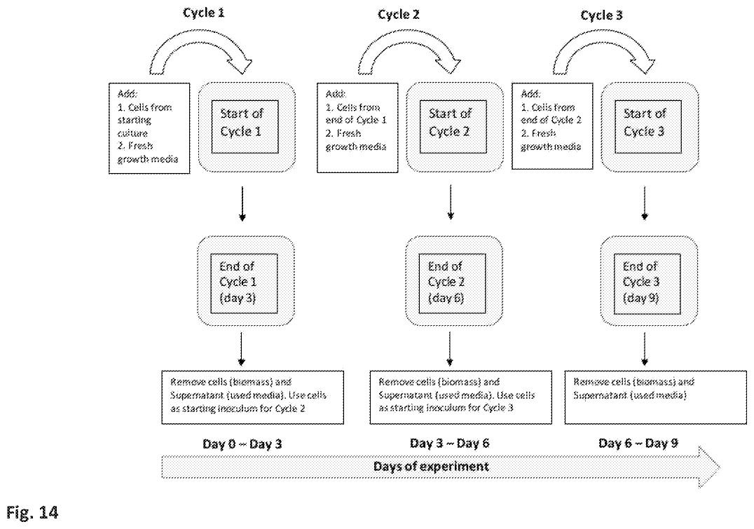

Condition 3: Cells from the end of Cycle 1 under Condition 1 were used as the inoculation cells introduced into fresh growth media. This incubation is referred to as Cycle 1 of Condition 3. 2 million cells/mL were inoculated into the fresh media and were grown for 3 days. Then for Cycle 2 the cells at the end of Cycle 1 were inoculated at 2 million cells/mL into fresh media and grown for 3 days. Cycle 3 was repeated the same way as Cycle 2. See FIG. 14.

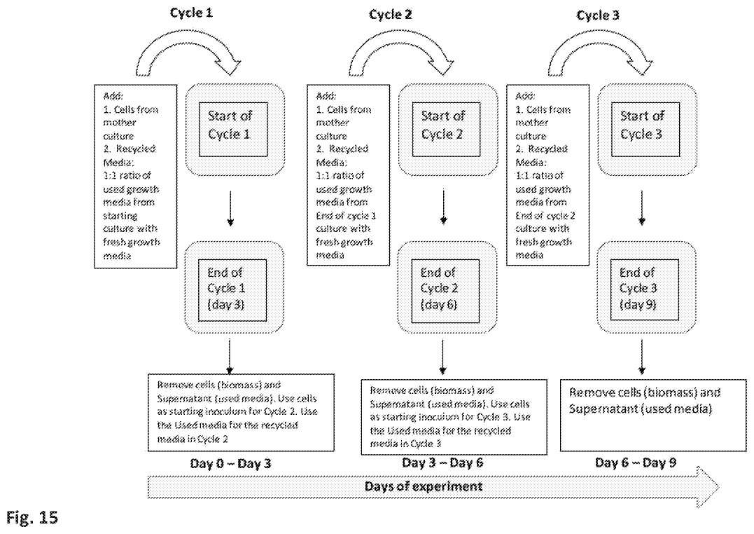

Condition 4: Cells from the end of Cycle 1 under Condition 1 were used as the inoculation cells introduced into recycled culture media (FIG. 15). The recycled growth media was made up of spent growth media and fresh growth media in a 1:1 ratio. Cells were inoculated at 2 million cells/mL and culture was grown for 3 days. For Cycle 2 the cells at the end of Cycle 1 were inoculated at 2 million cells/mL into recycled culture media comprising 1:1 mixture of spent growth media from Cycle 1 and fresh growth media. The culture was grown for 3 days. For Cycle 3, the spent growth media from Cycle 2 was mixed with fresh growth media at 1:1 ratio to generate a recycled culture media for Cycle 3. Cells from the end of Cycle 2 were inoculated (2 million cells/mL) into the recycled culture media and the cells were grown for 3 days. The skilled person can readily recognize that additional cycles can be carried out in a similar manner.

All cells were grown in the dark in 1 L vented cap flasks with a 400 mL working volume. Cells were incubated at 28° C. with shaking at 120 rpm. The dried biomass weight, dried supernatant weight and glucose consumption were measured for each condition and flask. All conditions were done in duplicate and each condition was grown for 3 consecutive cycles. Each cycle was 3 days and the total days past from start of Cycle 1 to end of Cycle 3 was 9 days.

Dried Biomass Weight

Dried biomass refers to biomass that has been freeze-dried in order to remove water molecules from the samples. The preparation of dried biomass was as described as in Example 1. The skilled person can readily recognize different methods suitable for drying biomass, for example, oven drying might be used. Dried cell biomass weight over time (days) of the culture is a measure of cell growth. Cell growth could be due to more cells i.e. replication or due to compositional changes in the cell i.e. generation of carbohydrates, protein or lipids within the cell.

Dred Supernatant Weight

Supernatant was removed from the pelleted cells in the example above by decanting it from the pelleted cells and freeze drying it. This process involves freezing the cell supernatant in a −80° C. for 10 min to 12 hours before putting the sample in a Freeze dryer under vacuum. This removes the frozen water molecules. What remains is the dried solutes that were left in the media. Solutes would be the compounds i.e. components from the media as well as potential excreted materials from the cells i.e. waste products. Over time, the solutes levels will decrease as the components of the media are used, for example glucose, the major carbon source.

Determining Efficiency

Conversion efficiency is a measure of media efficiency. Conversion efficiency is defined as the amount of biomass generated divided by the total amount of solutes consumed in the media. Biomass generated is calculated by taking the total mass of biomass at the end of the cycle and subtracting the initial total biomass in the culture at the start. The total amount of solutes consumed is calculated as the total of solutes in the culture at the start minus the total solutes on the last day. Conversion efficiency is determined as follows:

Conversion efficiency=(Total biomass generated at the end of a cycle/Total solutes consumed at the end of a cycle)*100%

Total Biomass Generated

Total biomass generate per cycle is determined as follows:

Total biomass generated per cycle=total dried biomass weight at the end of a cycle−total dried biomass weight at the beginning of a cycle

Total Solutes Consumed

Total solutes consumed per cycle is determined as follows:

Total solutes consumed per cycle=Initial solutes weight at the beginning of a cycle−final solutes weight at the end of a cycle

Overall Yield

Overall Yield is the measure of how much of the inputs were converted into biomass. In this calculation the amount of biomass generated in a cycle is determined by subtracting the dried biomass at the end of each cycle in grams by the initial dried biomass weight in gram from the start of the cycle. This is then divided by the total mass of inputs used in grams i.e. all the components that are in the growth media. Dried cell weight is defined as the dried biomass weight. Overall yield is determined as follows:

Overall Yield (gDCW/gInput)=total mass of biomass (dry cell weight) generated in the cycle (gDCW)/total mass of inputs used (gInput)

Supplements Yield

Supplement Yield is calculated similarly to Overall Yield, however, instead of the total mass of inputs used, it is the total mass in the hybrid media that is from the fresh media supplementation. In this calculation the amount of biomass generated in a cycle is determined by subtracting the dried biomass at the end of each cycle in grams by the initial dried biomass weight in gram from the start of the cycle. This is then divided by the total mass of supplemented inputs used in grams i.e. all the components that are in the fresh growth media that was added. Dried cell weight is defined as the dried biomass weight. Supplement Yield is determined as follows:

Supplement Yield (gDCW/gSInput)=total mass of generated biomass (dry cell weight) in the cycle (gDCW)/total mass of inputs from the fresh growth media used in the cycle (gSInput)

Yield Based on Glucose

As glucose is the major carbon source and makes up ⅔ of the media with respect to mass, the yield in terms of glucose utilization is also reported. This is defined as the dry biomass weight generated for a cycle divided by how much glucose was used in that cycle. This is measured either as a mass (i.e. grams) or by grams per liter (concentration) of culture or growth media. Yield based on glucose (concentration) is determined as follows:

Yield based on glucose (concentration)=(concentration of cells at the end of a cycle (g/L)−concentration of cells at start of cycle (g/L))/(concentration of glucose at the start of the cycle (g/L)−concentration of glucose at the end of the cycle (g/L)

Results and Discussions

Table 7 and Table 8 show summary supplement yield and yield based on glucose concentration from Euglena cells cultured under Condition 1 and Condition 2, respectively. DCW stands for the dried cell weight. gDCW is grams of Dried Cell weight. Each cycle's (Cycle 1-3) results are shown individually for supplement yield and yield based on glucose concentration. Several elements from each cycle are shown, the first being the final mass of biomass accumulated from each cycle. It also shows the supplement yield for all cycles based on the total mass generated in all three cycles divided by the sum of fresh growth media inputs for all three cycles. Finally, it also shows the average of the yields based on glucose.

Under Condition 1, the overall yield was highest on Cycle 1 and 2 at 0.26, while Cycle 3 was slightly lower (0.24). The yield based on the glucose concentration however is highest for Cycle 2 at 0.45 and lowest for Cycle 3 with 0.41. Under Condition 2, the supplement yield was highest on Cycle 1 at 0.40, while Cycles 2 and 3 remained lower (0.34 and 0.29 respectfully). The yield based on the glucose concentration however is highest for Cycle 1 at 0.61 and lowest for Cycle 3 with 0.45.

TABLE 7
Summary of Condition 1 Overall yield and yield based on glucose.

| | | | | | Overall | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gSInput) | (g/L) | Glucose (g/L)) |
| Cycle 1 | Day 0 | 1 | 0.4 | 12.26 | 0.26 | 21.68 | 0.44 |
| | Day 3 | 9.3 | 0.38 | | | 2.88 | |
| Cycle 2 | Day 0 | 1.2 | 0.41 | 12.26 | 0.26 | 20.36 | 0.45 |
| | Day 3 | 9.4 | 0.39 | | | 1.94 | |
| Cycle 3 | Day 0 | 2 | 0.42 | 12.26 | 0.24 | 19.84 | 0.41 |
| | Day 3 | 9.248 | 0.405 | | | 2.25 | |

| | | | | | |
|---|---|---|---|---|---|
| All Cycles | 11.46 g per 1.23 L | 36.78 | 0.25 | 54.81 | 0.43 |

TABLE 8
Summary of Condition 2's supplement yield
and yield based on glucose concentration.

| | | | | | Supplement | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gSInput) | (g/L) | Glucose (g/L)) |
| Cycle 1 | Day 0 | 0.9 | 0.4 | 6.13 | 0.40 | 10.88 | 0.61 |
| | Day 3 | 7.4 | 0.38 | | | 0.22 | |
| Cycle 2 | Day 0 | 2.1 | 0.41 | 6.13 | 0.34 | 10.32 | 0.54 |
| | Day 3 | 7.6 | 0.39 | | | 0.12 | |
| Cycle 3 | Day 0 | 1.8 | 0.42 | 6.13 | 0.29 | 9.92 | 0.45 |
| | Day 3 | 6.25 | 0.405 | | | 0.1 | |

| | | | | | |
|---|---|---|---|---|---|
| All Cycles | 8.70 g per 1.23 L | 18.39 | 0.34 | 30.67 | 0.54 |

When comparing Condition 1 (fresh growth media with mother culture cells) to Condition 2 (hybrid culture media containing 50% recycled growth media and 50% fresh media with mother culture cells), the supplement yield and yield based on glucose concentration is higher with the hybrid culture media. This indicates that the amount of biomass generated based on the total amount of supplemented inputs or glucose used is higher in Condition 2. Without wishing to be bound by theory, hybrid culture media has a higher yield may be due to the unique metabolism of the Euglena cell. “Waste” products that might be excreted by Euglena, such as acetic acid, lactic acid or succinic acid, may be able to metabolize and be useful as sources for growth. Without wishing to be bound by theory, as the hybrid culture media has more “Waste” product build up and as the main carbon source of glucose becomes limiting, the cell is able to use the “wastes” in the media in order to survive.

Table 9 and Table 10 shows summary overall yield, supplement yield and yield based on glucose concentration from Euglena cells cultured under from Condition 3 and Condition 4, respectively. DCW stands for the dried cell weight. gDCW is grams of Dried Cell weigh. Each cycle's (Cycle 1-3) results are shown individually for overall yield, supplement yield and yield based on glucose concentration. Several features from each cycle are noted, the first being the final mass of biomass accumulated from each cycle. Table 5 also shows the supplement yield for all cycles is based on the total mass generated in all three cycles divided by the sum of all inputs for all three cycles. Finally, the average of the yields based on glucose is also shown.

Under Condition 3, overall yield was highest on Cycle 1 at 0.28, while Cycles 2 and 3 remained lower (0.25). The yield based on the glucose concentration is highest for Cycle 1 at 0.49 and lowest for Cycle 3 with 0.42. Under Condition 4, supplement yield was highest on Cycle 1 at 0.42, while Cycles 2 and 3 remained similar (0.33 and 0.32 respectfully). The yield based on the glucose concentration however is highest for Cycle 1 at 0.68 and lowest for Cycle 3 with 0.48.

TABLE 9
Summary of Condition 3's overall yield and yield based on glucose concentration.

| | | | | | Overall | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gSInput) | (g/L) | Glucose (g/L)) |
| Cycle 1 | Day 0 | 1 | 0.4 | 12.26 | 0.28 | 21.16 | 0.49 |
| | Day 3 | 10.2 | 0.38 | | | 2.35 | |
| Cycle 2 | Day 0 | 2.3 | 0.41 | 12.26 | 0.25 | 20 | 0.44 |
| | Day 3 | 10.4 | 0.39 | | | 1.43 | |
| Cycle 3 | Day 0 | 1.8 | 0.42 | 12.26 | 0.25 | 20 | 0.42 |
| | Day 3 | 9.5 | 0.405 | | | 1.85 | |

| | | | | | |
|---|---|---|---|---|---|
| All Cycles | 12.33 g per 1.23 L | 36.78 | 0.26 | 55.53 | 0.45 |

TABLE 10
Summary of Condition 4's supplement yield
and yield based on glucose concentration.

| | | | | | Supplement | | Yield based |
|---|---|---|---|---|---|---|---|
| | | | Volume of | Total | Yield | | o glucose |
| | Age of | DCW | Culture | inputs | (gDCW/ | Glucose | (DCW (g/L))/ |
| Cycle | culture | (g/L) | (L) | (g) | gSInput) | (g/L) | Glucose (g/L)) |
| Cycle 1 | Day 0 | 0.95 | 0.4 | 6.13 | 0.42 | 10.28 | 0.68 |
| | Day 3 | 7.8 | 0.38 | | | 0.212 | |
| Cycle 2 | Day 0 | 2.3 | 0.41 | 6.13 | 0.33 | 9.76 | 0.55 |
| | Day 3 | 7.6 | 0.39 | | | 0.164 | |
| Cycle 3 | Day 0 | 1.8 | 0.42 | 6.13 | 0.32 | 10.4 | 0.48 |
| | Day 3 | 6.75 | 0.405 | | | 0.18 | |

| | | | | | |
|---|---|---|---|---|---|
| All Cycles | 6.24 g per 1.23 L | 18.39 | 0.36 | 29.884 | 0.57 |

When comparing Condition 3 (fresh growth media with reinoculated culture cells—i.e. cells from a previous culture other than the mother culture) to Condition 4 (hybrid culture media containing 50% recycled culture media and 50% fresh growth media, with reinoculated culture cells), the supplement yield (compared to the overall yield) and yield based on glucose concentration is higher with the hybrid culture media. This indicates that the amount of biomass generated based on the total amount of supplemented inputs or glucose used is higher in Condition 4. This is the same trend that was observed when comparing Condition 1 and 2. As the results between Condition 1 and 3 as well as Condition 2 and 4 only differ in the source of Euglena cells, it shows that the starting cell inoculum does not affect consumption of inputs. As there are difference between yield for both supplemented and glucose concentration between Conditions 1 and 3 with Conditions 2 and 4, the data shows that there is a factor present in the hybrid culture media that causes a higher conversion of the supplemented inputs into biomass. Without wishing to be bound by theory, unique metabolism of the Euglena cell may play a role in conversion, where “waste” products that Euglena excretes, such as acetic acid, lactic acid or succinic acid, are metabolized and used as sources for growth. Without wishing to be bound by theory, the hybrid culture media has more “waste” products built up and as the main carbon source of glucose becomes limiting, the cell is able to use the “wastes” in the media in order to survive.

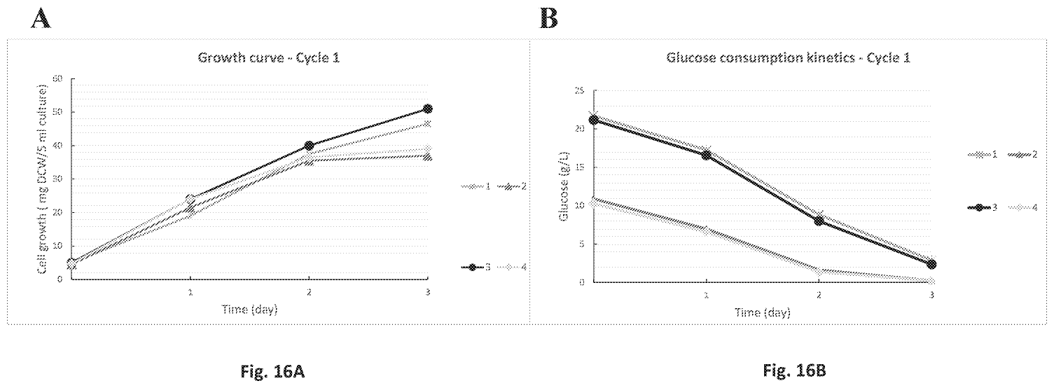

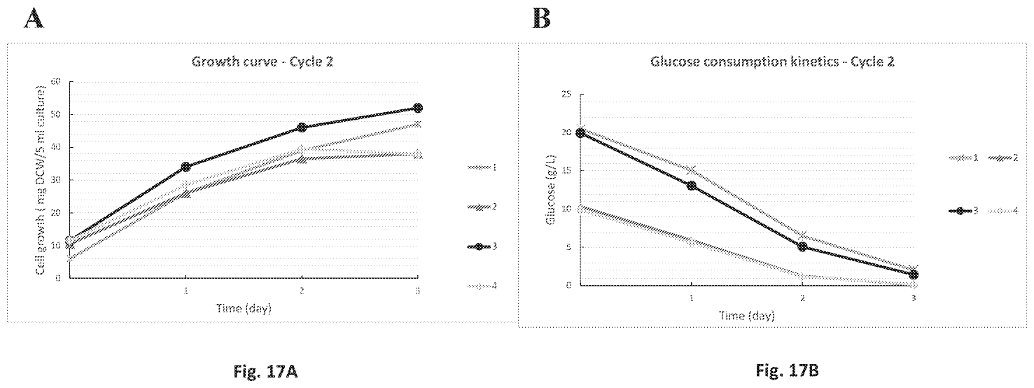

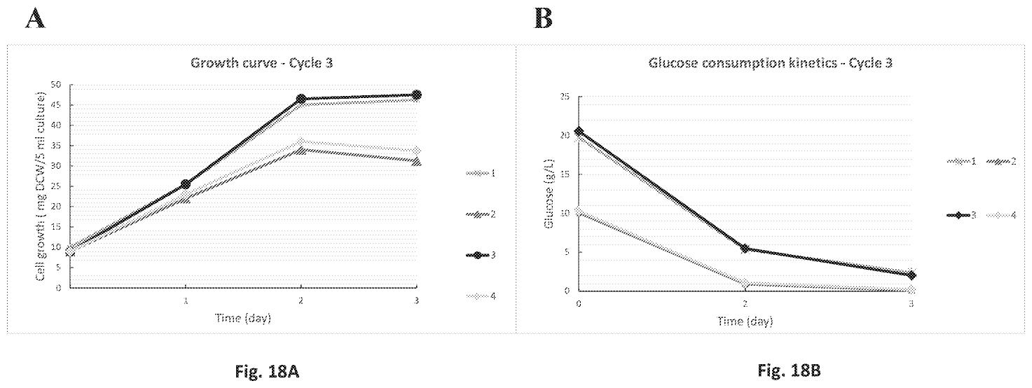

Similar trends are shown in FIGS. 16-18. These figures show growth curve and glucose consumption results for Cycles 1, 2 and 3. Panel A of these figures show that Conditions 2 and 4 have similar growth compared to Condition 1 and 3 up until day 2 for all three cycles. After day 2, Euglena growth under Conditions 2 and 4 was similar on day 3, but growth under Conditions 1 and 3 increases. In terms of glucose consumption (Panel B of FIGS. 16-18), Conditions 2 and 4 start with a lower glucose concentration (about half) than Conditions 1 and 3. As such, by day 2, Conditions 2 and 4 have consumed the majority of the glucose present in the hybrid growth media (<0.2 g/L). Under Conditions 1 and 3, by day 3 there is still approximately 2 g/L glucose remaining, indicating that not all the carbon source has been utilized by cells under these conditions.

Table 11 shows conversion efficiency summary. Conversion efficiencies (total biomass accumulated divided by the total amount of solutes in the source media consumed) are reported for each condition for each cycle. Conditions 1 and 3 show results from Euglena cells cultured under fresh media conditions, and Conditions 2 and 4 show results from Euglena cells under hybrid culture media conditions. The averages of conversion efficiencies for all cycles under each condition are shown.

TABLE 11
Conversion efficiency summary.

| Conversion | | | | | |
|---|---|---|---|---|---|
| Efficiency Summary | | Condition 1 | Condition 3 | Condition 2 | Condition 4 |
| Cycle 1 | Total biomass (mg) | 3153 | 3384 | 2509 | 2644.5 |
| | Total Solutes consumed (mg) | 6723 | 6965 | 5361 | 5806.5 |
| | Conversion efficiency (%) | 46.90 | 48.59 | 46.80 | 45.54 |
| Cycle 2 | Total biomass (mg) | 3239 | 3193 | 2165.5 | 2089 |
| | Total Solutes consumed (mg) | 6924.5 | 8013 | 5142.5 | 4524.5 |
| | Conversion efficiency (%) | 46.78 | 39.85 | 42.11 | 46.17 |
| Cycle 3 | Total biomass (mg) | 2808.75 | 2911.5 | 1579.25 | 1784.75 |
| | Total Solutes consumed (mg) | 10250.85 | 11556.3 | 7407.95 | 6787.95 |
| | Conversion efficiency (%) | 27.40 | 25.19 | 21.32 | 26.29 |
| All cycles | Average Conversion Efficiency (%) | 40.36 | 37.88 | 36.74 | 39.34 |

| | | |
|---|---|---|
| Average Fresh/Hybrid | 39.12 | 38.04 |
| Culture Media Conditions | | |

Conversion efficiency is a measure of media efficiency. Conversion efficiency is defined as the amount of biomass generated divided by the total amount of solutes consumed in the media. Solutes are defined as the components measured in the source media. In particular they are a measurement of what is in the source media after water is removed. As more biomass is generated, the higher the conversion efficiency. If a lot of solutes are consumed however and not a lot of biomass generated, the number will be lower. As such, as shown here, conversion efficiencies by Euglena cells grown in hybrid culture media are very similar to the fresh media's conversion efficiencies. When comparing the averages of fresh growth media to hybrid culture media, the conversion efficiencies are within 2% of each other (39% for fresh, 38% for recycled). If assuming fresh media was control efficiency, recycled media operated at a 97% efficiency overall.

In addition, the results show hybrid culture media having a higher conversion efficiency compared to fresh media. For example, in Cycle 2, Condition 2 and 4 had a higher conversion efficiency than Condition 3, and Condition 4 was similar to Condition 1 as well (both at 46%). In Cycle 3, Condition 4 had a slightly higher conversion efficiency than Condition 3. These results show that hybrid culture media-50% (50% recycled culture media and 50% fresh media) is not limiting the conversion of components in the source media into biomass.

TABLE 12
Dried cell weights of for a 4-day cycle of growth.

| Dry weight [g/L] | cycle 0 | cycle 1 | cycle 2 | cycle 3 | cycle 4 |
|---|---|---|---|---|---|
| Day 1 | 4.73 | 3.58 | 1.71 | 0.76 | 1.67 |
| Day 2 | 7.41 | 3.02 | 1.60 | 1.01 | 2.45 |
| Day 3 | 8.49 | 2.77 | 2.69 | 1.31 | 2.63 |
| Day 4 | 5.33 | 2.98 | 2.20 | 2.92 | 2.72 |

TABLE 13
Dried cell weights of for a 3-day cycle of growth.

| Dry weight [g/L] | cycle 0 | cycle 1 | cycle 2 | cycle 3 | cycle 4 |
|---|---|---|---|---|---|
| Day 1 | 2.86 | 4.22 | 3.86 | 3.32 | 2.54 |
| Day 2 | 4.67 | 4.72 | 3.72 | 4.30 | 13.09 |
| Day 3 | 5.46 | 5.17 | 3.49 | 6.41 | 7.64 |

Table 12 and Table 13 show the change in biomass with two different cycle lengths. A 3-day cycle shows a change in growth over time to the same degree as a 4-day cycle.

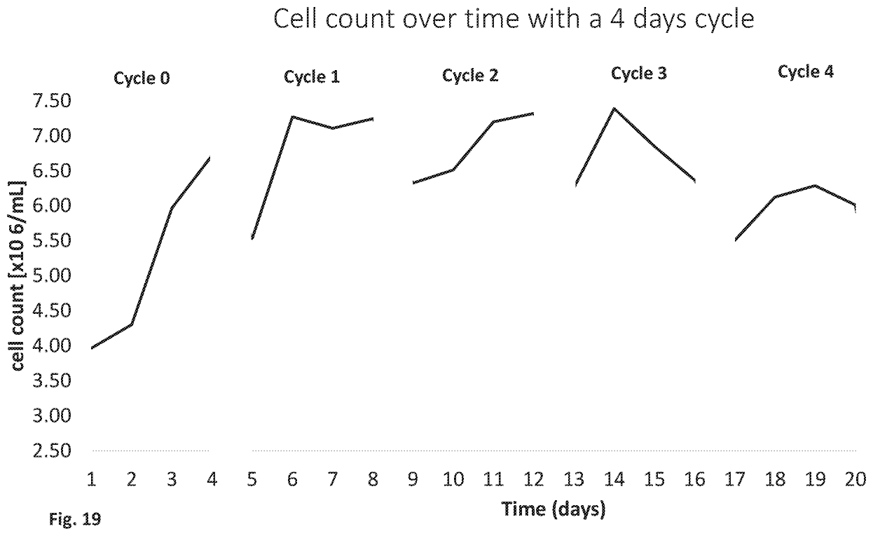

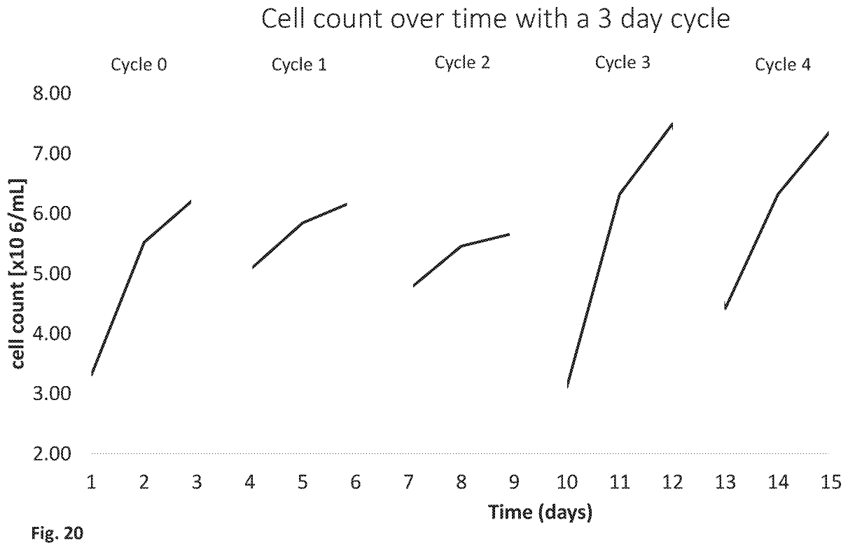

FIG. 19 and FIG. 20 show the difference between a 4-day growth cycle and a 3-day growth cycle. As well, FIG. 19 is an example of recycling media with a longer growth cycle and cells that were inoculated in a late to stationary phase. In FIG. 20, cells have a shorter growth cycle and are inoculated during more of an exponential phase.

### Example 3: Additional Studies Using Recycled Culture Media

### (a) Supplementation of Limiting Factor Glucose in Euglena Cultivation

A study is carried out to determine if glucose levels in the media are limiting to growth, conversion efficiency, and capacity of the media to be reused. Glucose is supplemented in the recycled culture media to bring the concentration back to the fresh growth media control. This is done at a flask scale over three, 3 day cycles.

### Methods & Materials

Euglena gracilis strain Z was used as the cell culture. Cells were inoculated into growth media as indicated in Table 14 which was autoclaved before use in culturing the cells. Three, 2 L baffled flasks with vented caps at a volume of 1 L were seeded at 2×10⁶ cells/mL and incubated with shaking under heterotrophic conditions at 28° C., 120 rpm. After 3 days growth media was separated under sterile conditions from the cell mass by centrifugation, and was combined together acting as the recycling media source for use in further cycles; thereby generating the first spent media that was used to make the recycled media hybrid for Cycle 1. Seed culture pellets were also combined and uniformly mixed before seeding Cycle 1 flasks.

TABLE 14
Fresh growth media used in Example 3 experiments,
pH adjusted to 3.2 and autoclaved.

| Compounds | Amount (g/L) | |
|---|---|---|
| Glucose | 15 | |
| Yeast Extract | 5 | |
| Ammonium Sulfate ((NH₄)₂SO₄) | 2 | |
| Potassium Phosphate (KH₂PO₄) | 1 | |
| Magnesium sulfate (MgSO₄•7H₂O) | 1 | |
| Calcium Chloride (CaCl₂•2H₂O) | 0.1 | |
| Ethylenedinitrilotetraacetic acid disodium salt | 0.05 | |
| dihydrate (Na₂EDTA•2H₂O) | | |
| Iron Chloride hexahydrate (FeCl₃•6H₂O) | 0.042 | |
| Zinc Sulphate heptahydrate (ZnSO₄•7H₂O) | 0.088 | |
| Manganese Chloride (MnCl₂•4H₂O) | 0.080 | |
| Copper Sulphate (CuSO₄•5H₂O) | 0.78 | mg/L |
| Boric Acid (H₃BO₃) | 0.57 | mg/L |
| Sodium Molybdate (Na₂MoO₄•2H₂O) | 0.004 | |
| Vitamin B₁ (Thiamine) | 0.01 | |
| Vitamin B₁₂ (Cyanocobalamin) | 0.05 | mg/L |
| Vitamin B₆ (Pyridoxine) | 0.002 | mg/L |
| Vitamin B₇ (Biotin) | 0.0001 | mg/L |

A source culture was also maintained heterotrophically in the media listed in Table 14 at 28° C., 120 rpm; throughout the experiment serving as an experimental mother or seed source for all treatments.

Conditions Tested

Experimental treatments were conducted in 1 L baffled flasks with vented caps at a culture volume of 500 mL. Cultures were incubated for heterotrophic growth with shaking in a Kuhner Shaker incubator at 28° C., 120 rpm for 3 days per cycle. Experimental treatments were seeded at approximately 2×10⁶ cells/mL. Conditions were done in n=2 and the total days past for the experiment, from the start of Cycle 1 to the end of Cycle 3 was 10 days. Measurements were taken at each day during the consecutive cycles where Day 3 of Cycle 1 was Day 0 of Cycle 2 etc. Harvesting and reuse of cell free spent media for use in recycled media hybrid was done under sterile conditions.

Experimental treatments included the following:

Condition 1: 100% fresh growth media where 500 mL of the media listed in Table 14 was inoculated with E. gracilis cells at 2×10⁶ cells/mL from the seed stock. Flasks were started with fresh media at the beginning of each cycle and reinoculated at the start of Cycle 2 and 3. Sample size n=2

Condition 2: Recycled media hybrid, where approximately 50% of the media source was obtained from cell free spent media from the previous growing cycle and 50% from fresh growth media and inoculated with 2×10⁶ cells/mL. This media was made of 1:1 ratio of spent growth media from the previous cycle and fresh growth media. For Cycle 2 the spent growth media from Cycle 1 was mixed with fresh growth media at a 1:1 ratio. Cycle 3 was repeated in the same way as Cycle 2. Cells were grown for 3 days. Sample size n=2.

Condition 3: Glucose supplemented 50% recycled media where approximately 50% of the media source was obtained from cell free spent media from the previous growing cycle, 50% was from fresh growth media and glucose was supplemented in the media at day 0 of each cycle using a concentrated glucose stock at 40% (w/v) to obtain an approximate 15 g/L of glucose under the assumption the recycled media had a glucose concentration of 0 g/L and 50% fresh media had a glucose concentration of 7.5 g/L. Approximately 9.375 mL of 40% (w/v) of concentrated glucose stock was added to this treatment at the start of each cycle. Sample size n=2

Cell Free Media

Cell free media was incubated under the same parameters as above in 250 mL baffled flasks with vented caps at a volume of 100 mL. 100% fresh media was maintained and refreshed at the beginning of each cycle. 50% recycled cell free media hybrid was obtained from the corresponding 50% recycled treatment flask of the previous cycle and mixed with fresh media in a 1:1 ratio i.e. 50% cell free media for cycle 2 was obtained from 50% recycled treatment in cycle 1. Cell free media was monitored and ensured to be free of cells at the beginning of each cycle by light microscopy, no growth was detected in any cell free media treatments. Each treatment was done in triplicate and the glucose levels as well as organic acids were measured to observe if there was any degradation during the experiment conditions.

Data Collection

Biomass and supernatant were harvested through centrifugation in 50 mL falcon tubes at 5000 rpm, 10 min, and the weights were recorded for various parameters and the corresponding dry weights following freeze drying. Glucose consumption was measured by a YSI analytical instrument; YSI 2950 by the same method as outlined in Example 1 and Example 2.

For No Cell controls/cell free media on Day 0 and 3 of each cycle the following measurements were taken under sterile conditions; 20 mL of culture was centrifuged at 5000 rpm, 5 mL was taken to determine supernatant dry weight following freeze drying, 1.5 mL for glucose analysis, 5 mL for organic acid profiling and the remainder for pH measurement. On Day 1 and 2, 7 mL was spun down at 5000 rpm, and used for glucose and pH measurements.

For Experimental treatments on Day 0 and 3, 25 mL of culture was aliquoted from each treatment under sterile conditions. Aliquot was used for cell count each day; 10 mL of culture was centrifuged at 5000 rpm and 5 mL removed for supernatant analysis followed by freeze drying. The remaining supernatant was removed, and pellet weighed followed by freeze drying. Remaining culture was centrifuged as above and 1.5 mL used for glucose analysis, 5 mL for organic acid profiling and the remainder for pH measurement. For Days 1 and 2, 8 mL of culture was removed under sterile conditions; 1 mL for cell count and the remainder centrifuged as above and 1.5 mL used for glucose and the remainder for pH measurements.

Dried Biomass weight was done as outlined in Example 2.

Dried supernatant weight was done as described in Example 2.

Further parameters that were determined in Example 2 were calculated as well.

Organic Acid Analysis

5 mL of supernatant was collected from each treatment and stored at −80 C until analysis could take place. 2 mL of sample were filtered through 0.2 um filter and 1 cc syringe into running vials.

Organic acid content was detected using HPLC. Agilent HPLC-1260 infinity system equipped with DAD and an Aminex HPLC Column of HPX-87H (300×8.7 mm) were used. The mobile phase was 5 mM sulfuric acid with a flow rate of 0.35 mL/min heated at 40 C. The DAD detector was set at 210 nm. 10 μL sample was directly injected after it was filtered through a 0.2 um syringe filter through an autosampler. Individual organic acid concentrations were calculated using calibration curves achieved from generated standard calibration curves using: Fumaric acid; Malate Standard for IC, Succinate Standard for IC, Pyruvic acid (Sigma Aldrich).

Results and Discussion

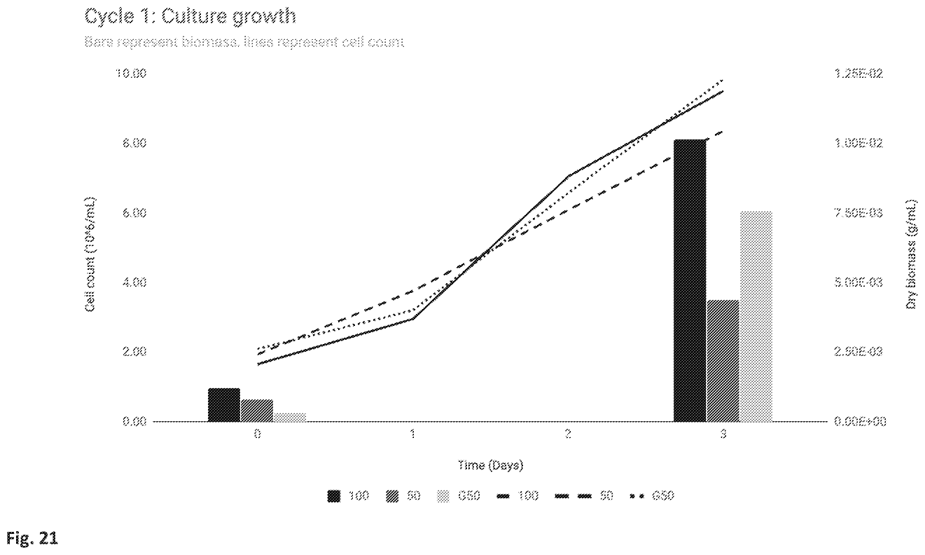

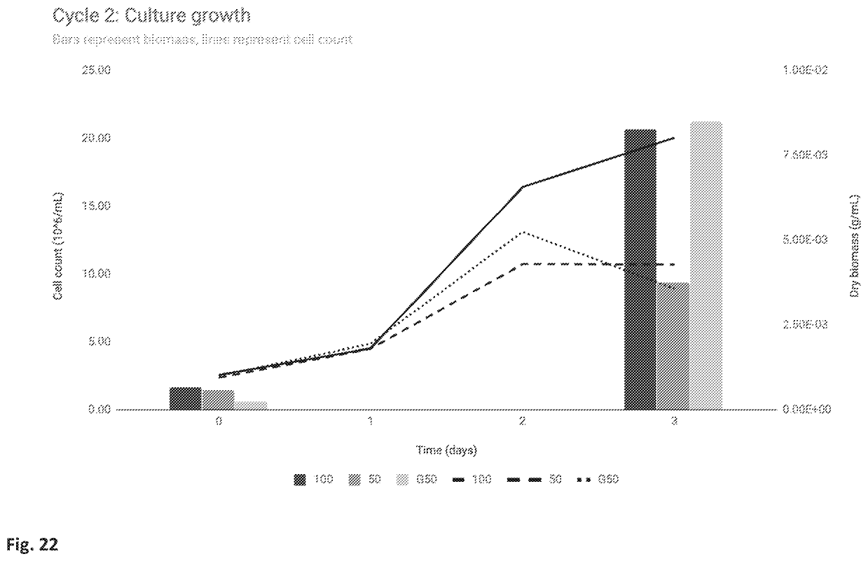

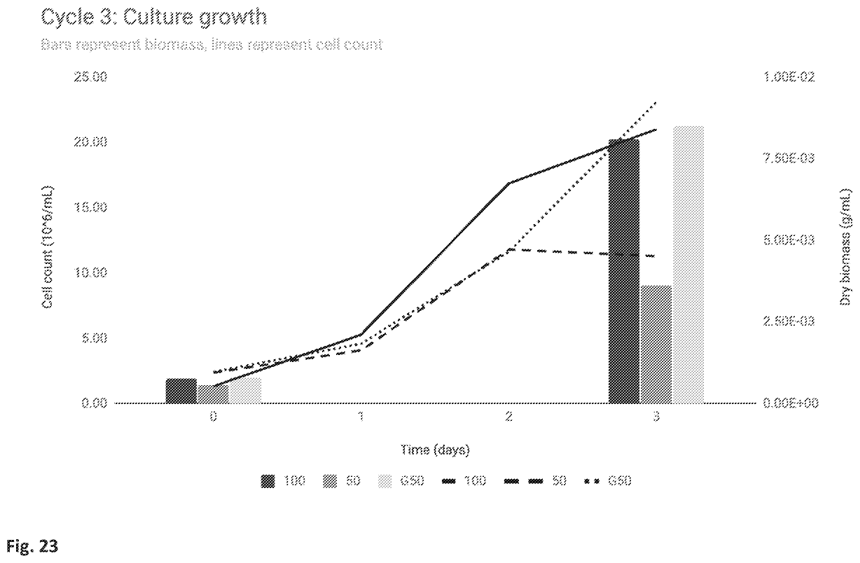

Culture growth during cycles was measured through both cell counting and dry cell weight (FIGS. 21-23). In cycle 1, there were similar cell counts across the samples (FIG. 21). During cycle 2 (FIG. 22), cell counts for the 50% recycled (50) and 50% recycled with supplemented glucose media (G50) (conditions 2 and 3) were reduced by the end of cycle relative to cell counts for the 100% fresh growth media (100) (condition 1). For Cycle 3 (FIG. 23), only the 50% recycled media (50) (condition 2) showed a reduction in cell count relative to cell counts for the 100% fresh growth media (100) (condition 1) by the end of the cycle. In terms of biomass, the glucose supplemented recycled media generated a similar amount of biomass as the 100% fresh growth media (11) over all cycles (FIGS. 21-23). The 50% recycled media generated about half the amount of biomass as the control over all cycles (FIGS. 21-23).

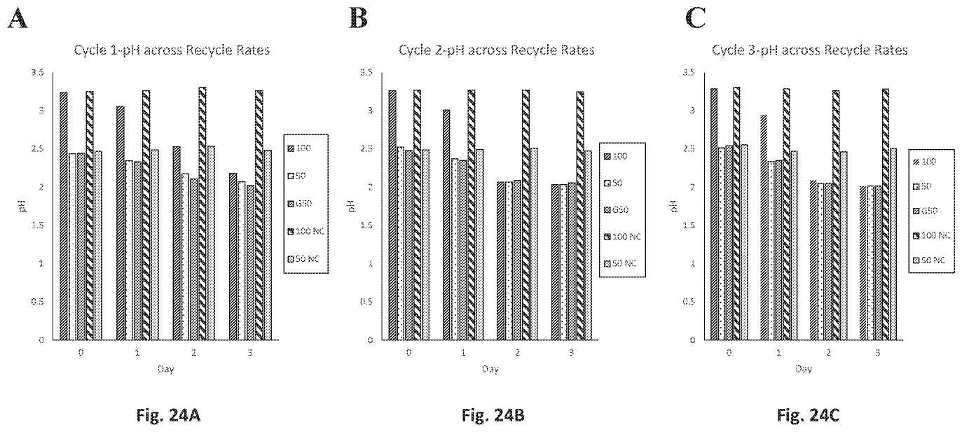

There were a few notable pH changes during the experiment (FIG. 24). The 100% fresh growth media condition (100) showed a higher pH than the 50% recycled (50) and glucose supplemented conditions (G50). The pH of the 100% fresh growth media decreased over time, while the pH of the 50% (50) and glucose supplemented recycled media (G50) conditions were more stable over time. There was little change in pH for both of the no cell controls (100 NC and 50 NC).

Conversion efficiency was calculated for all condition tested in the experiment (Table 15). On average, glucose supplemented media had the highest conversion efficiency (37%), as well as the highest in cycle 1 (41%). While the 50% recycled media had the lowest average over all cycles at 32%, this is due to the low cycle 2 conversion efficiency of 20%. In Cycle 1 and Cycle 3, the conversion efficiently of the 50% recycled media was 39%, which was higher than the 100% fresh media controls, and also higher than the average of all cycles for the glucose supplemented 50% recycled media sample. The 100% fresh growth media had comparable conversion efficiencies over all cycles with an average of 34. The average conversion efficiency of the 50% recycled media was 94% of that of the 100% fresh media and the average conversion efficiency of the glucose supplemented 50% recycled media was 109% of that of the 100% fresh media.

TABLE 15
Conversion efficiency summary of Example 3a conditions.

| | | | | 50% Recycled |
|---|---|---|---|---|
| Conversion | | 100% Fresh | 50% Recycled | Media Glucose |
| Efficiency Summary | | Media | Media | Supplemented |
| Cycle 1 | Total biomass (mg) | 3459 | 1503 | 3173 |
| | Total Solutes consumed (mg) | 9231 | 3892 | 7776 |
| | Conversion efficiency (%) | 37 | 39 | 41 |
| Cycle 2 | Total biomass (mg) | 3178 | 1078 | 3641 |
| | Total Solutes consumed (mg) | 9631 | 5395 | 10661 |
| | Conversion efficiency (%) | 33 | 20 | 34 |
| Cycle 3 | Total biomass (mg) | 2962 | 1391 | 3350 |
| | Total Solutes consumed (mg) | 9295 | 3596 | 9677 |
| | Conversion efficiency (%) | 32 | 39 | 35 |
| All cycles | Average Conversion Efficiency (%) | 34 | 32 | 37 |

TABLE 16
Summary of 100% fresh growth media overall yield
and yield based on glucose concentration.

| | | | | | Overall | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gInput) | (g/L) | Glucose (g/L)) |
| Cycle 1 | Day 0 | 1.35 | 0.5 | 12.19 | 0.284 | 16.31 | 0.53 |
| | Day 3 | 9.537 | 0.434 | | | 0.8 | |
| Cycle 2 | Day 0 | 0.74 | 0.5 | 12.19 | 0.261 | 13.61 | 0.55 |
| | Day 3 | 8.175 | 0.434 | | | 0.01 | |
| Cycle 3 | Day 0 | 0.844 | 0.5 | 12.19 | 0.243 | 15.1 | 0.46 |
| | Day 3 | 7.798 | 0.434 | | | 0.01 | |

| | | | | | |
|---|---|---|---|---|---|
| All Cycles | 25.51 g in 1.302 L | 36.56 | 0.263 | 44.2 | 0.51 |

TABLE 17
Summary of 50% recycled media supplement yield
and yield based on glucose concentration.

| | | | | | Supplement | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gInput) | (g/L) | Glucose (g/L)) |
| Cycle 1 | Day 0 | 0.8 | 0.500 | 6.09 | 0.247 | 7.87 | 0.48 |
| | Day 3 | 4.384 | 0.434 | | | 0.41 | |
| Cycle 2 | Day 0 | 0.825 | 0.500 | 6.09 | 0.177 | 7.17 | 0.37 |
| | Day 3 | 3.435 | 0.434 | | | 0.06 | |
| Cycle 3 | Day 0 | 0.526 | 0.500 | 6.09 | 0.239 | 7.57 | 0.46 |
| | Day 3 | 3.9655 | 0.434 | | | 0.05 | |

| | | | | | |
|---|---|---|---|---|---|
| All Cycles | 11.7845 in 1.302 L | 18.28 | 0.221 | 22.09 | 0.43 |

TABLE 18
Summary of glucose supplement 50% recycled media supplement
yield and yield based on glucose concentration.

| | | | | | Supplement | | Yield based |
|---|---|---|---|---|---|---|---|
| | | | Volume of | Total | Yield | | on glucose |
| | Age of | DCW | Culture | inputs | (gDCW/ | Glucose | (DCW (g/L))/ |
| Cycle | culture | (g/L) | (L) | (g) | gInput) | (g/L) | Glucose (g/L)) |
| Cycle 1 | Day 0 | 0.37 | 0.509 | 9.84 | 0.322 | 16.31 | 0.53 |
| | Day 3 | 7.588 | 0.443 | | | 2.61 | |
| Cycle 2 | Day 0 | 0.235 | 0.509 | 9.84 | 0.370 | 13.61 | 0.69 |
| | Day 3 | 8.49 | 0.443 | | | 1.59 | |
| Cycle 3 | Day 0 | 0.807 | 0.509 | 9.84 | 0.340 | 16.08 | 0.49 |
| | Day 3 | 8.49 | 0.443 | | | 0.24 | |

| | | | | | |
|---|---|---|---|---|---|
| All Cycles | 24.57 g in 1.329 L | 29.53 | 0.344 | 41.55 | 0.57 |

Tables 16-18 highlight the overall yield, supplement yield and yield based on glucose which shows similar trends to the conversion efficiency. Glucose supplement hybrid media had the highest supplement yield and highest yield based on glucose with 0.344 and 0.57 respectively. 100% fresh media had the next highest at 0.263 and 0.51 respectively for overall yield and yield based on glucose. Hybrid media at a 50% recycled rate had the lowest supplement yield at 0.221 and 0.43 from yield based on glucose. Cycle 2 was low for the hybrid media across all measurements, however cycle 1 and 3 were more comparable to the average seen in the 100% fresh media. Example 3b was conducted to look further into the effects of the glucose supplementation on biomass generation.

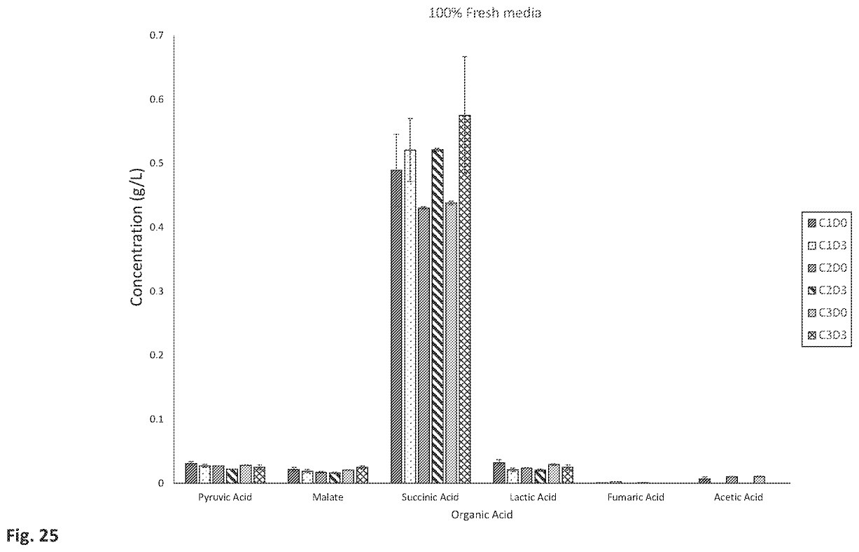

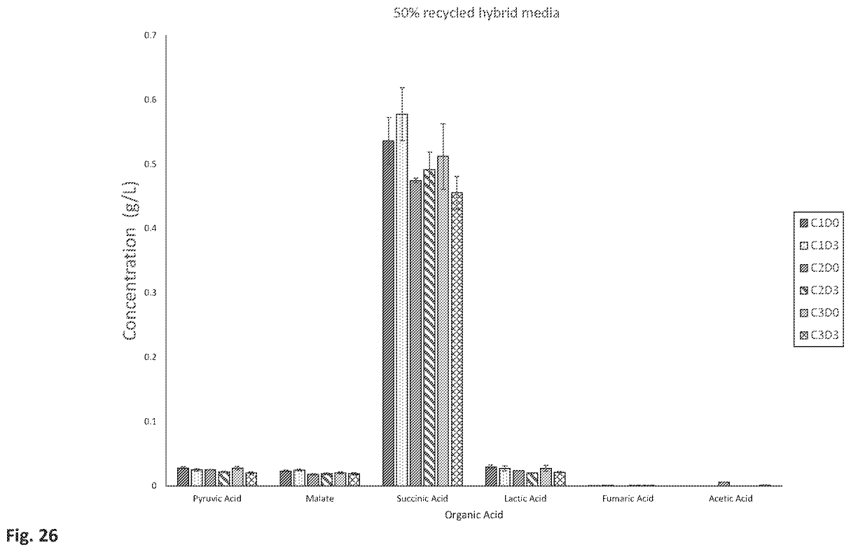

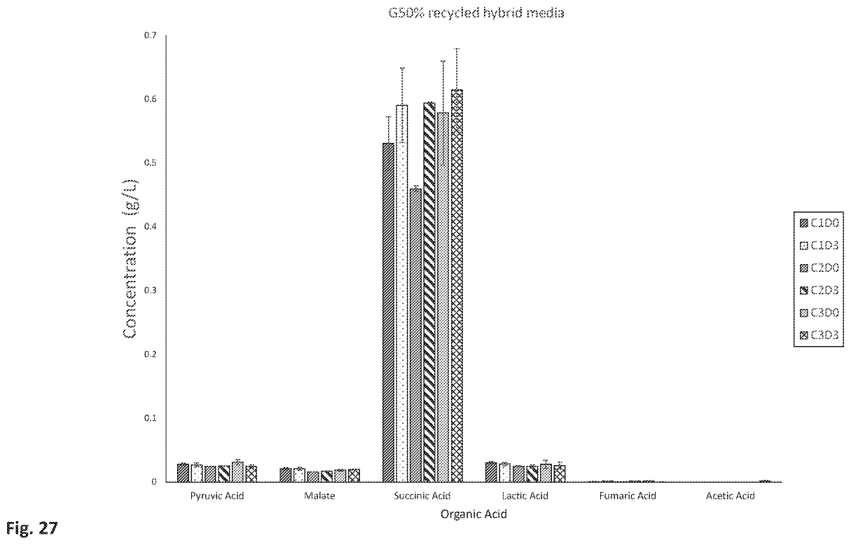

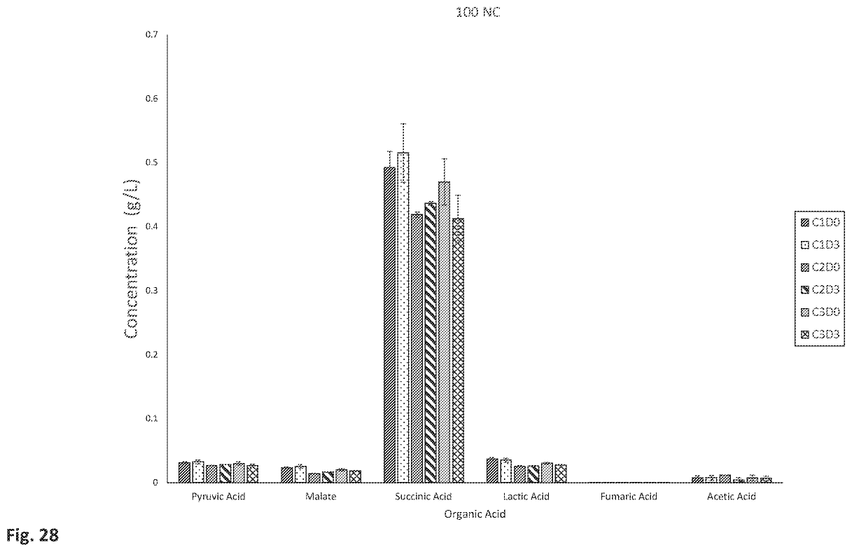

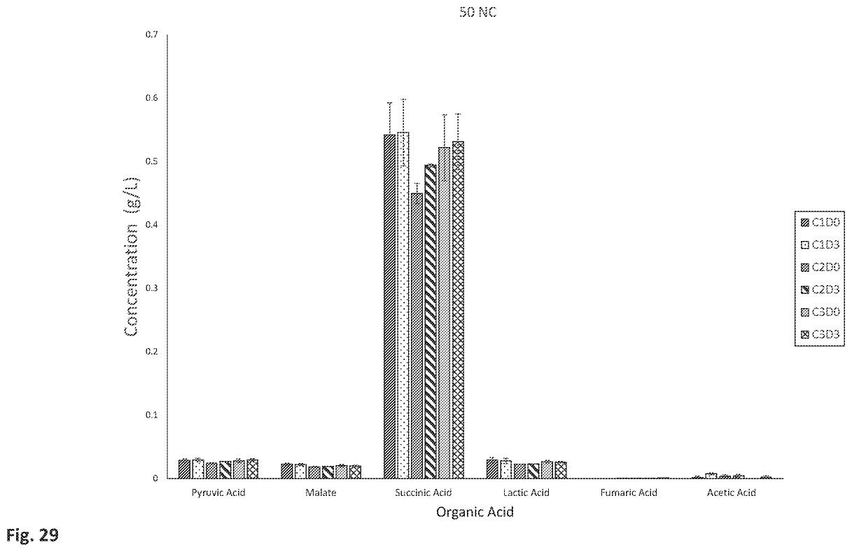

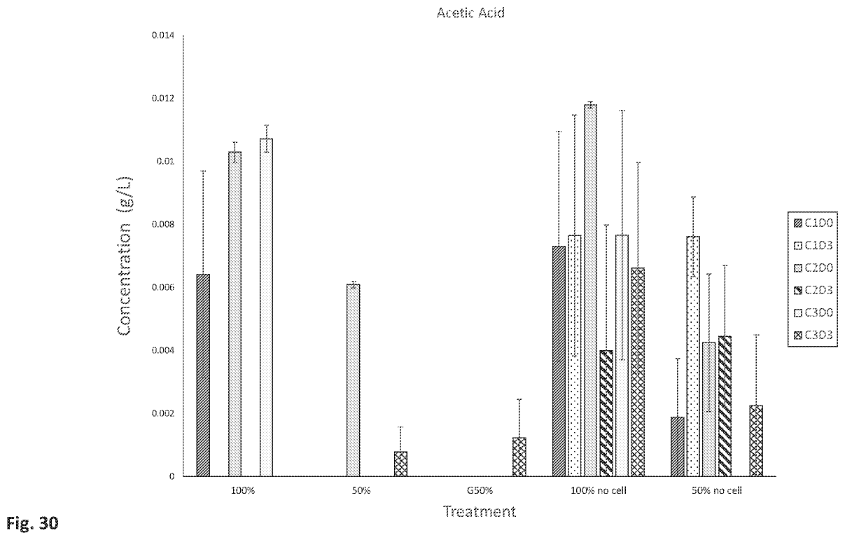

Organic acid analysis was conducted on all sample types, as well as the no cell controls to see if there was degradation of the acids in the media over the time points. 6 organic acids were investigated based on literature and available standards: Pyruvic acid, malate, succinic acid, lactic acid, fumaric acid, and acetic acid. FIGS. 30-35 highlights all organic acids per experimental condition, where FIG. 25 represents the 100% fresh growth media control, FIG. 26 represents the 50% recycled media sample, FIG. 27 represents the glucose supplemented 50% recycled media, FIG. 28 represents the 100% no cell control and FIG. 29 presents the 50% recycled media no cell control. Clear trends indicate that succinic acid is highest in all conditions, and fumaric acid and acetic acid are the lower excreted organic acids in the media. FIGS. 30-35 show the organic acids over all samples. Acetic acid (FIG. 30) has an interesting trend, where in the 100% fresh growth media control it is present at the start of each cycle and is not detected by the end of the cycle. In cycle 2 for the 50% recycled media there was presence of acetic acid at the beginning however, non at the end of the cycle. There was also acetic acid detected at the end of cycle 3 in both 50% recycled and glucose supplemented recycled media, however, taking into account standard error, these levels are at very low levels. In general the no cell controls showed similar levels of acetic acids at the start and end of each cycle, and further supports the levels seen in the 100% samples and cycle 2 50% recycled media as Euglena actively using the organic acids.

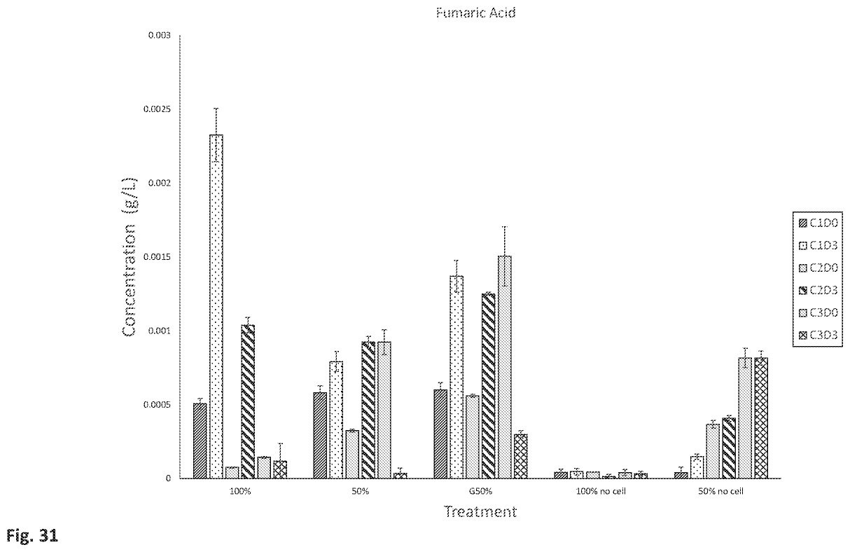

FIG. 31 shows fumaric acid in all treatments and the no cell controls. No cell controls show consistent levels within each cycle. The 100% fresh growth media sample, in cycle 1 and 2 there is a large increase in fumaric excreted into the media, whereas there is little change in cycle 3. For 50% recycled media Cycle 1 has a slight increase in fumaric by the end of the cycle, a larger increase is seen in cycle 2 and cycle 3 has a drastic decrease of the level of fumaric acid. The glucose supplemented 50% recycled media shows similar trend to the 50% recycled media.

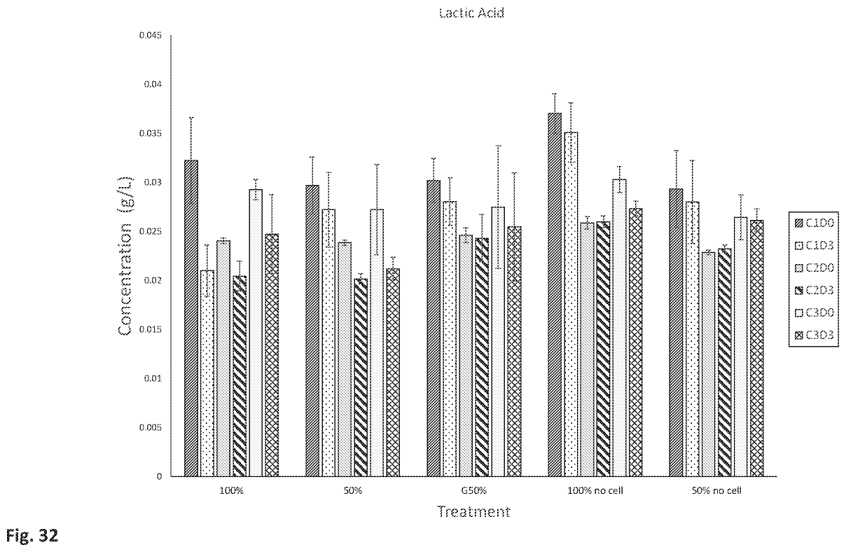

FIG. 32 shows lactic acid results. Similar levels of lactic acid were seen in both the no cell controls and the experimental conditions. Of note however, 100% fresh growth media in cycle 1 showed a decrease in lactic acid at the end of the cycle that was not reflected in the no cell control. Cycle 3, day 3 in the 50% recycled media example might also show the same slight decrease. These results indicate that in general Euglena was not uptaking lactic acid from the media, with just 2 examples on where there was slight decreases in lactic acid levels. As well, as lactic acid levels are not increasing in the media at the end of each cycle this suggests that the culture did not go into anaerobic state or metabolism as lactic acid is a common by product of anaerobic respiration.

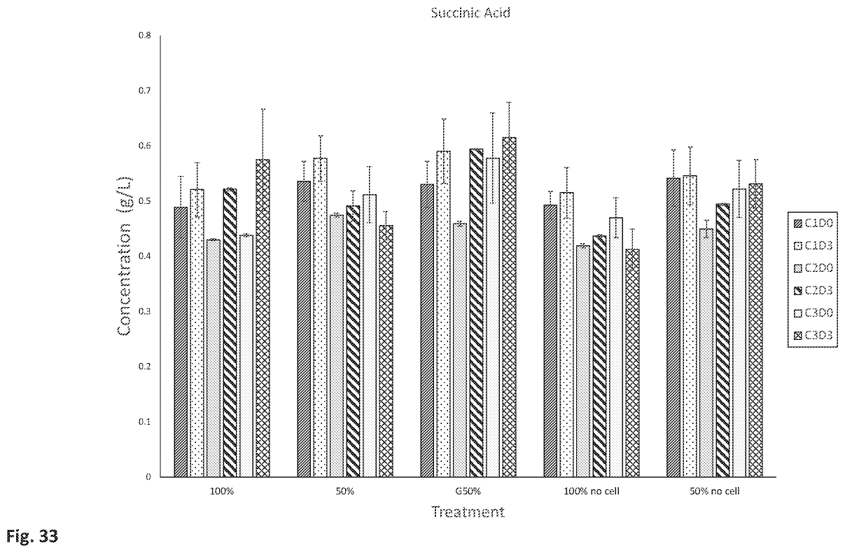

FIG. 33 represents succinic acid levels in the conditions, which had high levels observed in all treatments, including the no cell controls. Cycle 2 and 3 for in the 100% fresh growth media there was an increase in succinic acid at the end of the cycle. There was slight decrease in the organic acid at the end of cycle 3 for 50% recycled media. Cycle 2 in the 50% recycled media with glucose supplementation showed an increase in the organic acid at the end of the cycle.

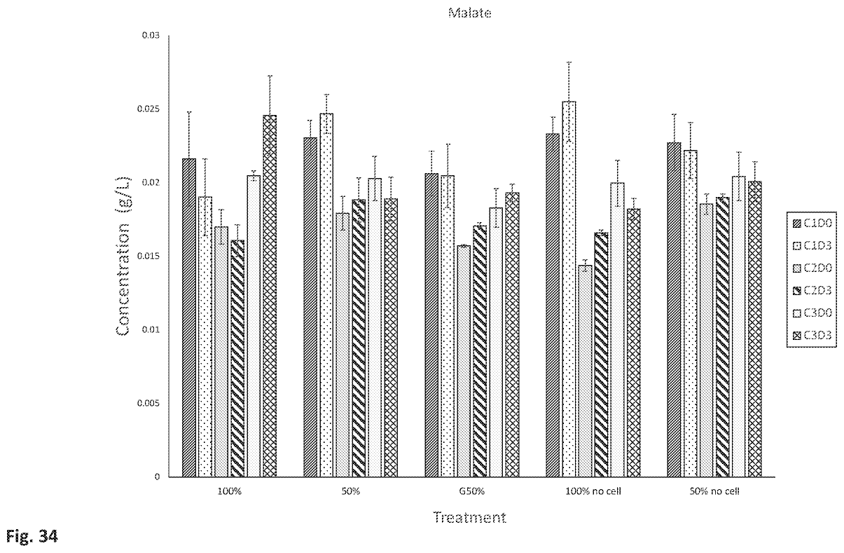

FIG. 34 shows malate levels in all treatments. There is a slight increase in malate at the end of cycle 3 in the 100% fresh growth media treatment. In the 50% recycled media there was an increase in malate at the end of cycle 1 and there was no observable differences seen in the glucose supplemented 50% recycled media.

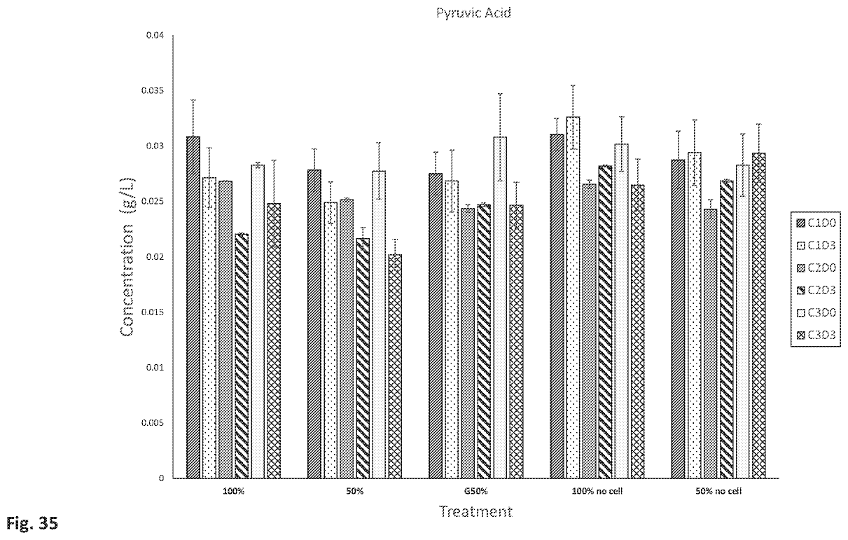

Lastly, FIG. 35 shows the pyruvate acid levels in all samples. In general, the majority of experimental conditions show a decrease in pyruvate at the end of each cycle. As the no cell controls had small changes where the pyruvate levels slightly increased, this suggests that Euglena is metabolizing the pyruvate acid. Pyruvate is a carbon source that has been used in growth of Euglena gracilis under heterotrophic and conditions. Pyruvate is a commonly used molecule in Euglena metabolism, and can be used under aerobic conditions, cytosolic pyruvate is directly imported into the mitochondria for energy use in the Kreb's cycle, or can be converted first to lactate in the cytosol and then imported into the mitochondria.

There's evidence that there are several groupings of extracellular metabolites being excreted into the medium: phytohormones, organic acids etc. We know from the above results that E. gracilis is capable of excreting organic acids as well as potentially utilizing them during growth. Succinate and Lactate are commonly used as a source for bioplastics; given the growing conditions used there is an abundance of succinic acid in the organic acid profile. This organic acid could potentially be isolated and used for further processing and applications.

In addition, no inhibitory effects were observed from organic acid accumulation or presence in the media of any treatment. This shows that organic acid accumulation over 3 cycles is not an inhibitory factor for biomass accumulation or substrate utilization.

### (b) Comparison of Control Media

In this study, the effect of the media changes, in particular the solute input influence on the overall Euglena biomass production and conversion efficiency. As well, it is investigated if the addition to glucose only is boosting the growth back to equivalent with fresh media or is the hybrid media influencing it as well.

Methods and Materials

Base media reported in Table 14 was used for the 100% fresh media control. To compare lower amounts of solutes, a 50% fresh media solution was made which has half the inputs, as seen below in Table 19. for a control for the glucose supplement hybrid media, a full amount of glucose as compared to the 100% fresh media control as seen in Table 19. 50% hybrid recycled media and glucose supplemented hybrid media were generated as described in Example 3a for 1 cycle.

TABLE 19
100%, 50% and glucose supplemented 50% fresh growth media
used in Example 3b. pH adjusted to 3.2 and autoclaved.

| | | | | | Glucose supplemented | |
|---|---|---|---|---|---|---|
| | 100% Fresh | | 50% Fresh | | 50% Fresh | |
| Compounds | Media (g/L) | | Media (g/L) | | Media (g/L) | |
| Glucose | 15 | | 7.5 | | 15 | |
| Yeast Extract | 5 | | 2.5 | | 2.5 | |
| Ammonium Sulfate ((NH₄)₂SO₄) | 2 | | 1 | | 1 | |
| Potassium Phosphate (KH₂PO₄) | 1 | | 0.5 | | 0.5 | |
| Magnesium sulfate (MgSO₄•7H₂O) | 1 | | 0.5 | | 0.5 | |
| Calcium Chloride (CaCl₂•2H₂O) | 0.1 | | 0.05 | | 0.05 | |
| Ethylenedinitrilotetraacetic acid disodium salt | 0.05 | | 0.025 | | 0.025 | |
| dihydrate (Na₂EDTA•2H₂O) | | | | | | |
| Iron Chloride hexahydrate (FeCl₃•6H₂O) | 0.042 | | 0.021 | | 0.021 | |
| Zinc Sulphate heptahydrate (ZnSO₄•7H₂O) | 0.088 | | 0.044 | | 0.044 | |
| Manganese Chloride (MnCl₂•4H₂O) | 0.080 | | 0.040 | | 0.040 | |
| Copper Sulphate (CuSO₄•5H₂O) | 0.78 | mg/L | 0.39 | mg/L | 0.39 | mg/L |
| Boric Acid (H₃BO₃) | 0.57 | mg/L | 0.285 | mg/L | 0.285 | mg/L |
| Sodium Molybdate (Na₂MoO₄•2H₂O) | 0.004 | | 0.002 | | 0.002 | |
| Vitamin B₁ (Thiamine) | 0.01 | | 0.005 | | 0.005 | |
| Vitamin B₁₂ (Cyanocobalamin) | 0.05 | mg/L | 0.025 | mg/L | 0.025 | mg/L |
| Vitamin B₆ (Pyridoxine) | 0.002 | mg/L | 0.001 | mg/L | 0.001 | mg/L |
| Vitamin B₇ (Biotin) | 0.0001 | mg/L | 0.00005 | mg/L | 0.00005 | mg/L |

Experimental treatments were conducted in 250 mL flasks with vented caps at a culture volume of 100 mL. Cultures were incubated for heterotrophic growth with shaking in a Kuhner Shaker incubator at 28° C., 120 rpm for 3 days for one cycle. Experimental treatments were seeded at approximately 2×10⁶ cells/mL. Conditions were done in n=2.

Biomass and supernatant were harvested through centrifugation in 50 mL falcon tubes at 5000 rpm, 10 min, and the weights were recorded for various parameters and the corresponding dry weights following freeze drying. Glucose consumption was measured by a YSI analytical instrument; YSI 2950 by the same method as outlined in Example 1 and Example 2.

For samples on Day 0 and 3, 12 mL of culture was aliquoted from each treatment under sterile conditions. Aliquot was used for cell count each day; 11 mL of culture was centrifuged at 5000 rpm and 5 mL removed for supernatant analysis followed by freeze drying. The remaining supernatant was removed, and pellet weighed followed by freeze drying. Remaining culture was centrifuged as above and 1.5 mL used for glucose analysis, 5 mL for organic acid profiling and the remainder for pH measurement. For Days 1 and 2, 7 mL of culture was removed under sterile conditions; 1 mL for cell count and the remainder centrifuged as above and 1.5 mL used for glucose and the remainder for pH measurements.

Dried Biomass weight was done as outlined in Example 2.

Dried supernatant weight was done as described in Example 2.

Further parameters that were determined in Example 2 were calculated as well.

Results and Discussion

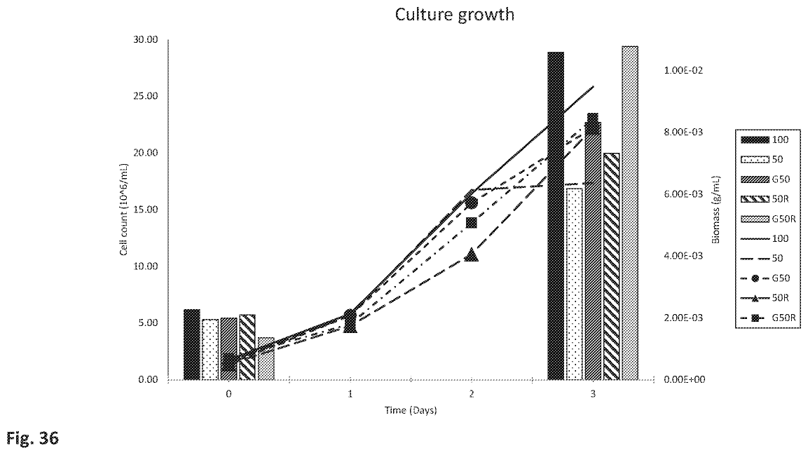

Cell growth parameters were measured for cell count and cell biomass in FIG. 36. For 100% fresh growth media, cell count was highest and biomass accumulation over the cycle was one of the highest. The 50% fresh growth media had the lowest cell count over the 3 day cycle, and the lowest amount of biomass generated. Glucose supplement 50% fresh growth media showed a higher cell count and biomass generation on day 3 than the 50% fresh growth media, however it was not as high as the 100% fresh growth media control. When comparing the 50% hybrid media, there was increase cell count and biomass for the hybrid media compared to the 50% fresh media control. In terms of the glucose supplemented hybrid media to the glucose supplemented 50% fresh growth media, the cell count was similar to the fresh media control. However, the biomass generation was the highest observed in all samples even 100% fresh media control. As the biomass generation was higher and comparable to the 100% fresh growth media growth, this is suggesting that the recycling component was needed in order to have similar biomass generation. A similar trend was observed for the 50% hybrid media compared to the 50% fresh media control, however it was not to the same extent as the glucose supplemented 50% hybrid media.

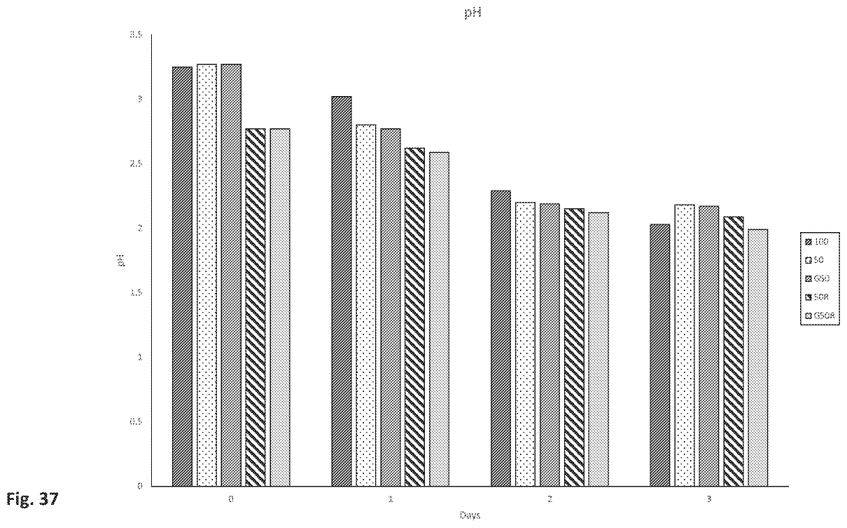

pH measurements are observed in FIG. 37. as seen in example 3a, the pH for the 100% fresh growth media decreased over time and had the highest pH decrease across all samples. The 50% fresh media and glucose supplemented 50% fresh media had a similar trend to the 100% fresh growth media. The two hybrid medias started at a lower pH as they contained spent media that was at a lower pH level, which resulted in a smaller decrease in pH. Overall, by day 3 the pH of all cultures was the same.

Table 20 highlights the conversion efficiency of the different conditions tested in example 3b. Overall, glucose supplemented hybrid media had the highest conversion efficiency at 22.9%. This is higher that the 100% fresh growth media control (13.7%), 50% fresh media (15.3%), glucose supplemented 50% fresh growth media (15.9%), and 50% Hybrid Media (13.9%). As the efficiency is higher than the glucose supplemented 50% fresh media, this suggests that that the recycled media has an effect on the conversion efficiency. As it is higher than the 50% Hybrid Media which is also with recycled media, this suggests that it is the combined effect of the higher glucose and recycled media that has a positive effect on the conversion efficiency. 50% Hybrid Media is also comparable to the 100% fresh media control sample, while the other fresh media controls (50% and glucose supplemented) are slightly higher.

TABLE 20
Summary of conversion efficiency for Example 3b.
Conversion Efficiency Summary Example 3b

| | | |
|---|---|---|
| 100% Fresh Media | Total biomass (mg) | 429.4 |
| | Total Solutes consumed (mg) | 3136.6 |
| | Conversion efficiency (%) | 13.7 |
| 50% Fresh Media | Total biomass (mg) | 187.8 |
| | Total Solutes consumed (mg) | 1231.0 |
| | Conversion efficiency (%) | 15.3 |
| Glucose Supplemented | Total biomass (mg) | 328.4 |
| 50% Fresh Media | Total Solutes consumed (mg) | 2061.8 |
| | Conversion efficiency (%) | 15.9 |
| 50% Hybrid Media | Total biomass (mg) | 244.6 |
| | Total Solutes consumed (mg) | 1757.8 |
| | Conversion efficiency (%) | 13.9 |
| Glucose Supplemented | Total biomass (mg) | 550.4 |
| 50% Hybrid Media | Total Solutes consumed (mg) | 2403.6 |
| | Conversion efficiency (%) | 22.9 |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of culturing a Euglena sp. microorganism, Schizochytrium sp. microorganism, or a Chlorella sp. microorganism comprising:
culturing the microorganism in a hybrid culture media;
maintaining the microorganism heterotrophically in an environment substantially free from light, or entirely no light;
wherein the hybrid culture media comprises a carbon source, optionally a carbohydrate; and
wherein the hybrid culture media comprises fresh media and recycled culture media.

2. The method of claim 1, wherein the microorganism is inoculated at about 1×10⁵ cells/mL to about 5×10⁷ cells/mL.

3. The method of claim 1, wherein the microorganism is inoculated at about 0.5 g/L to about 150 g/L dry cell weight.

4. The method of any one of claims 1-3, wherein the recycled culture media is obtained by separating the recycled culture media from a source culture media, wherein the source culture media is in a lag phase, an exponential phase, or a stationary phase.

5. The method of any one of claims 1-3, wherein the microorganism is grown for about 4 hours to about 350 hours, or up to about 75 days.

6. The method of any one of claims 1-5, wherein the microorganism is grown for at least 2, 3, 4, 5, 6, 7, 8, 9, 10, 11, 12, 13, 14, 15, 16, 17, 18, 19, 20, 25, 30, 35, 40, 45 or 50 cycles.

7. The method of any one of claims 1-6, wherein the method is batch, fed-batch, semi-continuous or continuous.

8. The method of claim 7, wherein the method is fed-batch, semi-continuous or continuous.

9. The method of claim 7, wherein the method is semi-continuous or continuous.

10. The method of claim 8 or 9, wherein recycled culture media is added to the hybrid culture media at lag, exponential or stationary phase.

11. The method of claim 10, wherein the recycled culture media is selected from the group consisting of a culture media, a feed media, a spent media, a supplemented media, and combinations thereof, optionally a spent media or a supplemented media.

12. The method of any one of claims 1-11, wherein the microorganism is harvested at lag, exponential, or stationary phase.

13. The method of claim 12, wherein the harvested microorganism are separated from the media and the media is recycled back into the culture.

14. The method of claim 13, wherein the media recycled back into the culture is spent media.

15. The method of claim 14, wherein the spent media comprises total carbohydrate of less than about 3, 2.5, 2, 1.5, 1, 0.5, 0.4, 0.3, 0.2, 0.1, 0.01 g/L.

16. The method of claim 15, wherein the carbohydrate is glucose, fructose, galactose, lactose, maltose, sucrose, molasses, glycerol, xylose, dextrose, honey, and/or corn syrup.

17. The method of any one of claims 14-16, wherein about 10% to about 75% of the spent media is returned to the culture, optionally about 25% to about 75%, optional about 50% to about 75%, optionally about 75%.

18. The method of any one of claims 1-17, wherein the microorganism is selected from the group consisting of Euglena gracilis, Euglena sanguinea, Euglena deses, Euglena mutabilis, Euglena acus, Euglena virdis, Euglena anabaena, Euglena geniculata, Euglena oxyuris, Euglena proxima, Euglena tipteris, Euglena chiamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabica, Euglena granulata, Euglena obtusa, Euglena limnophila, Euglena hemichromata, Euglena vaiabilis, Euglena caudata, Euglena minima, Euglena communis, Euglena magnifica, Euglena terricola, Euglena velata, Euglena repulsans, Euglena clavata, Euglena lata, Euglena tuberculata, Euglena contabrica, Euglena ascusformis, Euglena ostendensis, Chlorella autotrophica, Chlorella colonials, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella vaiabilis, Chlorella volutis, Chlorella vulgaris, Schizochytrium aggregatum, Schizochytrium limacinum, Schizochytrium minutum, and combinations thereof.

19. The method of any one of claims 1-18, wherein the method does not comprise phototrophic culturing.

20. The method of any one of claims 1-19, wherein the method does not comprise hybrid media sterilization.

21. The method of any one of claims 1-20, wherein the microorganism is Euglena and the method of culturing Euglena produces about 0.05 nmol/mL to about 100 nmol/mL cytokinins, optionally about 0.05 nmol/mL to about 75 nmol/mL cytokinins, optionally about 0.05 nmol/mL to about 50 nmol/mL cytokinins, optionally about 0.05 nmol/mL to about 40 nmol/mL cytokinins, optionally about 0.05 nmol/mL to about 35 nmol/mL cytokinins, optionally about 0.05 nmol/mL to about 30 nmol/mL cytokinins, optionally about 0.05 nmol/mL to about 25 nmol/mL cytokinins, optionally about 0.05 nmol/mL to about 20 nmol/mL cytokinins, optionally about 0.05 nmol/mL to about 15 nmol/mL cytokinins, optionally about 0.05 nmol/mL to about 10 nmol/mL cytokinins, optionally about 0.05 nmol/mL to about 5 nmol/mL cytokinins, optionally about 0.05 nmol/mL to about 4 nmol/mL cytokinins, optionally about 0.05 nmol/mL to about 3 nmol/mL cytokinins, optionally about 0.05 nmol/mL to about 2.5 nmol/mL cytokinins, optionally about 0.05 nmol/mL to about 2 nmol/mL cytokinins, optionally about 0.05 nmol/mL to about 1.5 nmol/mL cytokinins, optionally about 0.05 nmol/mL to about 1 nmol/mL cytokinins, optionally about 0.05 nmol/mL to about 0.5 nmol/mL cytokinins, optionally about 0.05 nmol/mL to about 0.1 nmol/mL cytokinins, optionally about 0.05 nmol/mL to about 25 nmol/mL cytokinins.

22. The method of any one of claims 1-21, wherein the microorganism is Euglena and the method of culturing Euglena produces about 0.01 pmol/mL to about 100 nmol/mL ABA.

23. The method of any one of claims 1-22, wherein the microorganism is Euglena and the method of culturing Euglena produces about 0.000005 g/L to about 20 g/L of an organic acid selected from the group consisting of citric acid, citrate, fumaric acid, fumarate, malic acid, malate, pyruvic acid, pyruvate, succinic acid, succinate, acetic acid, acetate, lactic acid, lactate, and combinations thereof.

24. The method of any one of claims 1-23, wherein the culture media is maintained at a pH of between about 2.5 to about 5, optionally between about 2.5 to about 4.

25. The method of any one of claims 1-24, wherein the culture media maintains a relative conversion efficiency of at least 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%.

26. A method for producing a recycled culture media suitable for heterotrophically culturing a Euglena sp. microorganism, Schizochytrium sp. microorganism, or a Chlorella sp microorganism, comprising:
culturing the microorganism in a culture media comprising a carbohydrate;
maintaining an environment with substantially, or entirely no light;
producing recycled culture media;
separating recycled culture media from cells; and
collecting recycled culture media;
wherein the microorganism is cultured until the carbohydrate is below 3 g/L;
wherein the microorganism is Euglena gracilis;
wherein the recycled culture media is obtained by separating the recycled culture media from a source culture media;
wherein the source culture media is in a lag phase, an exponential phase, or a stationary phase; and
wherein the source culture media is a hybrid culture media or a stock culture media.

27. The method of claim 26, wherein the recycled culture media comprises total carbohydrate of less than about 3, 2.5, 2, 1.5, 1, 0.5, 0.4, 0.3, 0.2, 0.1, 0.01 g/L.

28. The method of claim 26 or 27, wherein the carbohydrate is glucose, fructose, galactose, lactose, maltose, sucrose, molasses, glycerol, xylose, dextrose, honey, corn syrup, or combinations thereof.

29. A culture media comprising a hybrid culture media, wherein the hybrid culture media comprises a carbohydrate; and wherein the hybrid culture media comprises fresh media and recycled culture media.

30. The culture media of claim 29, wherein the carbohydrate is glucose, fructose, galactose, lactose, maltose, sucrose, molasses, glycerol, xylose, dextrose, honey, corn syrup, or combinations thereof.

31. The culture media of claim 29 or 30, wherein the culture media comprises about 0.01 pmol/mL to about 100 nmol/mL cytokinins.

32. The culture media of any one of claims 29-31, wherein the culture media comprises about 0.01 pmol/mL to about 100 nmol/mL ABA.

33. The culture media of any one of claims 29-32, wherein the culture media comprises about 0.000005 g/L to about 20 g/L of an organic acid selected from the group consisting of citric acid, citrate, fumaric acid, fumarate, malic acid, malate, pyruvic acid, pyruvate, succinic acid, succinate, acetic acid, acetate, lactic acid, lactate, and combinations thereof.

34. The culture media of any one of claims 29-33, wherein the hybrid culture media comprises about 10%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 99.99% recycled culture media.

35. A use of the culture media produced by the method of any one of claims 29-34 for culturing a microorganism.
